SEQUENCE LISTING (51 sequences)

SEQ ID NO: 1 - protein, 329 aa, synthetic construct
MLLLVTSLLLCELPHPAFLLIPVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 436 aa, synthetic construct
MLLLVTSLLLCELPHPAFLLIPVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 546 aa, synthetic construct
MLLLVTSLLLCELPHPAFLLIPVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 22 aa, synthetic construct
MLLLVTSLLLCELPHPAFLLIP

SEQ ID NO: 5 - protein, 113 aa, synthetic construct
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 6 - protein, 92 aa, synthetic construct
DSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 7 - protein, 12 aa, Homo sapiens
ESKYGPPCPPCP

SEQ ID NO: 8 - protein, 110 aa, Homo sapiens
APEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 9 - protein, 107 aa, Homo sapiens
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 28 aa, Homo sapiens
MFWVLVVVGGVLACYSLLVTVAFIIFWV

SEQ ID NO: 11 - protein, 42 aa, Homo sapiens
KRGRKKLLYIFKQPFMRPVQTTQEEDGCSCRFPEEEEGGCEL

SEQ ID NO: 12 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 41 aa, Homo sapiens
RSKRSRGGHSDYMNMTPRRPGPTRKHYQPYAPPRDFAAYRS

SEQ ID NO: 14 - protein, 14 aa, synthetic construct
AHIVMVDAYKPTKG

SEQ ID NO: 15 - protein, 17 aa, synthetic construct
  MOD_RES 1: note = D-arginine
  MOD_RES 14: note = D-arginine
RGVPHIVMVDAYKRYKG

SEQ ID NO: 16 - protein, 21 aa, synthetic construct
  SITE 1: note = acetylated asparagine
  MOD_RES 4: note = Hydroxyproline
  MOD_RES 14: note = D-alanine
  MOD_RES 19: note = D-arginine
  MOD_RES 21: note = D-lysine
NCVPNLRGDLQVLAQKVCRTK

SEQ ID NO: 17 - protein, 14 aa, synthetic construct
  SITE 1: note = acetylated alanine
AHIVMDVAYKPTKK

SEQ ID NO: 18 - protein, 18 aa, synthetic construct
  MOD_RES 1: note = D-arginine
  MOD_RES 14: note = D-arginine
  MOD_RES 18: note = D-lysine
RGVPHIVMVDAYKRYKGK

SEQ ID NO: 19 - protein, 20 aa, synthetic construct
NAVPNLRGDLQVLAQKVART

SEQ ID NO: 20 - protein, 16 aa, synthetic construct
RGVPHIVMVDAYKRYK

SEQ ID NO: 21 - protein, 328 aa, synthetic construct
MLLLVTSLLLCELPHPAFLLIPVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTESKYGPPCPPCPMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 435 aa, synthetic construct
MLLLVTSLLLCELPHPAFLLIPVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 92 aa, synthetic construct
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 24 - protein, 24 aa, Thosea asigna virus
LEGGGEGRGSLLTCGDVEENPGPR

SEQ ID NO: 25 - protein, 21 aa, Thosea asigna virus
GSGEGRGSLLTCGDVEENPGP

SEQ ID NO: 26 - protein, 22 aa, Teschovirus
GSGATNFSLLKQAGDVEENPGP

SEQ ID NO: 27 - protein, 23 aa, Equine rhinitis A virus
GSGQCTNYALLKLAGDVESNPGP

SEQ ID NO: 28 - protein, 25 aa, Foot-and-mouth disease virus
GSGVKQTLNFDLLKLAGDVESNPGP

SEQ ID NO: 29 - protein, 545 aa, synthetic construct
MLLLVTSLLLCELPHPAFLLIPVTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHTESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 449 aa, synthetic construct
MLLLVTSLLLCELPHPAFLLIPRGVPHIVMVDAYKRYKESKYGPPCPPCPAPEFDGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 17 aa, synthetic construct
  MOD_RES 1: note = D-arginine
  MOD_RES 5: note = D-histidine
  MOD_RES 14: note = D-arginine
  MOD_RES 16: note = D-lysine
RGVPHIVMVDAYKRYKG

SEQ ID NO: 32 - protein, 17 aa, synthetic construct
  MOD_RES 1: note = D-arginine
  MOD_RES 3: note = D-valine
  MOD_RES 9: note = D-valine
  MOD_RES 14: note = D-arginine
RGVPHIVMVDAYKRYKG

SEQ ID NO: 33 - protein, 17 aa, synthetic construct
  MOD_RES 1: note = D-arginine
  MOD_RES 5: note = D-histidine
  MOD_RES 14: note = D-arginine
RGVPHIVMVDAYKRYKG

SEQ ID NO: 34 - protein, 17 aa, synthetic construct
  MOD_RES 1: note = D-arginine
  MOD_RES 14: note = D-arginine
  MOD_RES 16: note = D-lysine
RGVPHIVMVDAYKRYKG

SEQ ID NO: 35 - protein, 17 aa, synthetic construct
  MOD_RES 1: note = D-arginine
  MOD_RES 3: note = D-valine
  MOD_RES 5: note = D-histidine
  MOD_RES 7: note = D-valine
  MOD_RES 9: note = D-valine
  MOD_RES 14: note = D-arginine
RGVPHIVMVDAYKRYKG

SEQ ID NO: 36 - protein, 17 aa, synthetic construct
  MOD_RES 1: note = D-arginine
  MOD_RES 3: note = D-valine
  MOD_RES 14: note = D-arginine
RGVPHIVMVDAYKRYKG

SEQ ID NO: 37 - protein, 129 aa, synthetic construct
DCDIPTTENLYFQGAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 38 - protein, 5 aa, synthetic construct
  REGION 1..5: note = The entire sequence of amino acids 1-5 can be repeated one or more times.
GGGGS

SEQ ID NO: 39 - protein, 5 aa, synthetic construct
  REGION 1..5: note = The entire sequence of amino acids 1-5 can be repeated one or more times
EAAAK

SEQ ID NO: 40 - protein, 6 aa, synthetic construct
HHHHHH

SEQ ID NO: 41 - protein, 8 aa, synthetic construct
DYKDDDDK

SEQ ID NO: 42 - protein, 8 aa, synthetic construct
DLYDDDDK

SEQ ID NO: 43 - protein, 15 aa, synthetic construct
GLNDIFEAQKIEWHE

SEQ ID NO: 44 - protein, 26 aa, synthetic construct
KRRWKKNFIAVSAANRFKKISSSGAL

SEQ ID NO: 45 - protein, 9 aa, synthetic construct
YPYDVPDYA

SEQ ID NO: 46 - protein, 10 aa, synthetic construct
EQKLISEEDL

SEQ ID NO: 47 - protein, 8 aa, synthetic construct
WRHPQFGG

SEQ ID NO: 48 - protein, 8 aa, synthetic construct
WSHPQFEK

SEQ ID NO: 49 - protein, 13 aa, synthetic construct
SLAELLNAGLGGS

SEQ ID NO: 50 - protein, 8 aa, synthetic construct
TQDPSRVG

SEQ ID NO: 51 - protein, 14 aa, synthetic construct
GKPIPNPLLGLDST

(19) United States
(12) Patent Application Publication
(10) Pub. No.: US 2026/0345776 A1
(43) Pub. Date: Nov. 5, 2026

(54) SYNTHETIC INTERMEDIATES FOR UNIVERSAL CHIMERIC ANTIGEN RECEPTOR IMMUNE CELL THERAPIES

(71) Applicants: University of Washington, Seattle, WA (US); Seattle Children's Hospital d/b/a Seattle Children's Research Institute, Seattle, WA (US)

(72) Inventors: Drew Sellers, Seattle, WA (US); Ian Cardle, Seattle, WA (US)

(73) Assignees: University of Washington, Seattle, WA (US); Seattle Children's Hospital d/b/a Seattle Children's Research Institute, Seattle, WA (US)

(21) Appl. No.: 19/120,635

(22) PCT Filed: Oct. 12, 2023
(86) PCT No.: PCT/US2023/076755

§ 371 Date: Apr. 11, 2025

Related U.S. Application Data
(60) Provisional application No. 63/379,624, filed on Oct. 14, 2022.

(51) Int. Cl. A61K 40/31; A61K 40/11; A61K 40/42; A61K 47/54; A61K 47/62; A61P 35/00

(52) U.S. Cl. CPC A61K 40/31; A61K 40/11; A61K 40/4204; A61K 47/549; A61K 47/62; A61P 35/00

(57) ABSTRACT

Example compositions, systems, devices, and methods are used for universal chimeric antigen receptor T (UCAR-T) cell therapy. An example method includes administering, to a subject, engineered immune cells that express a chimeric antigen receptor (CAR) including an extracellular component and an intracellular component linked by a transmembrane domain, wherein the extracellular component includes an intermediary-binding domain. The example method further includes administering, to the subject, a synthetic intermediary including a tag linked to an antigen binding domain, the tag specifically binding the intermediary-binding domain, the antigen binding domain specifically binding an antigen expressed on a surface of a target cell in the subject.

### CROSS-REFERENCE TO RELATED APPLICATION(S)

This application is a U.S. National Phase Patent Application based on International Patent Application No. PCT/US2023/076755, filed on Oct. 12, 2023, which claims the priority of U.S. Provisional App. No. 63/379,624, filed on Oct. 14, 2022, each of which is incorporated by reference herein in its entirety.

### STATEMENT REGARDING FEDERALLY SPONSORED RESEARCH OR DEVELOPMENT

This invention was made with government support under Grant No. R01NS118247, awarded by the National Institute of Neurological Disorders and Stroke and Grant No. R01AG063845, awarded by the National Institute on Aging and Grant No. DGE-1762114, awarded by the National Science Foundation. The government has certain rights in the invention.

### REFERENCE TO SEQUENCE LISTING

The Sequence Listing associated with this application is provided in XML format in lieu of a paper copy and is hereby incorporated by reference into the specification. The name of the file containing the Sequence Listing is 3EL5980.XML. The file is 65,536 bytes, was created on Apr. 1, 2025, and is being submitted electronically via Patent Center.

### TECHNICAL FIELD

This application relates to universal chimeric antigen receptor (CAR) immune-cell therapies, and more particularly, to synthetic intermediaries for CAR immune-cell therapies.

### BACKGROUND

Significant progress has been made in genetically engineering cells of the immune system to target and kill unwanted cell types, such as cancer cells. Many of these immune cells are T-cells that have been genetically engineered to express a CAR. CARs are proteins including several distinct subcomponents that allow the genetically modified T-cells to recognize and kill cancer cells. The subcomponents include at least an extracellular component and an intracellular component. The extracellular component includes a binding domain that specifically binds a marker (e.g., an antigen) that is preferentially present on the surface of unwanted cells, such as cancer cells. When the binding domain binds such markers, the intracellular component signals the T-cell to destroy the bound cell. CAR can additionally include a transmembrane domain that can link the extracellular component to the intracellular component.

The advent of CAR T-cell therapy has significantly changed the cancer treatment landscape, prompting a transition from one-size-fits-all chemotherapy to personalized therapies tailored to a patient's cells. There have been multiple CAR T-cell therapies approved by the United States Food and Drug Administration (FDA) for treating CD19⁺ or BCMA⁺ hematological malignancies (Maude, et al., NEJM 2018, 378 (5), 439-448; Schuster, et al., NEJM 2018, 380 (1), 45-56; Neelapu, et al., NEJM 2017, 377 (26), 2531-2544; Locke et al., The Lancet Oncology 2019, 20 (1), 31-42; Wang et al., NEJM 2020, 382 (14), 1331-1342; Abramson, et al., The Lancet 2020, 396 (10254), 839-852; Munshi, et al. NEJM 2021, 384 (8), 705-716; Berdeja, et al., Frontiers in Oncology 2020, 10, 849). Leading up to their FDA approval, these therapies resulted in 33-67% complete response rates in patients and follow-up real-world outcomes have tracked similarly. This success has led to the pursuit of other indications for these treatments as well as clinical investigation of CAR T-cells for targeting solid tumors (Martinez, et al., Frontiers in Immunology 2019, 10, 128; Bagley, et al., Pharmacology & Therapeutics 2020, 205, 107419).

Despite the favorable outcomes of current CAR T-cell therapies, they have significant faults. As many as 45% of patients that have complete responses to CD19-directed CAR T-cell therapy will eventually relapse, many with CD19-negative disease (Xu, et al., Frontiers in Immunology 2019, 10, 2664; Gardner, et al., Blood 2017, 129 (25), 3322-3331). Permanent loss of CD19 from the cell surface is thought to occur by many means, including alternative splicing (Sotillo, et al., Cancer Discovery 2015, 5 (12), 1282-1295), frameshift mutations (Orlando, et al., Nature Medicine 2018, 24 (10), 1504-1506), lineage switching (Gardner, et al., Blood 2016, 127 (20), 2406-2410), and because therapeutic pressure from CAR treatment drives enrichment of tumor clones with these traits. Importantly, these CD19-negative clones can exist at the onset of disease (Fischer, et al., J. of Immunotherapy 2017, 40 (5), 187-195), suggesting that hematological malignancies are more antigenically complex than initially surmised. The majority of patients receiving these therapies have been observed to experience some degree of cytokine release syndrome (CRS) and neurotoxicity, and a quarter of patients will present with severe, grade 3 or higher symptoms (Santomasso, et al., The Other Side of CAR T-Cell Therapy: Cytokine Release Syndrome, Neurologic Toxicity, and Financial Burden. American Society of Clinical Oncology Educational Book 2019, No. 39, 433-444; Gust, et al., CNS Drugs 2018, 32 (12), 1091-1101). These side effects are a consequence of on-target CAR T-cell activation, as evidenced by their association with higher pre-infusion tumor burden and higher peak CAR T-cell expansion (Teachey, et al., Cancer Discovery 2016, 6 (6), 664-679; Santomasso, et al., Cancer Discovery 2018, 8 (8), 958-971).

These drawbacks highlight the inflexible design of CAR T-cell therapies used in the clinic. Conventional CARs are composed of an extracellular single-chain variable fragment (scFv) fused to intracellular signaling and costimulatory domains, and T-cell activation occurs when the scFv domain binds to a target antigen on an opposing cell. While highly specific, the single-target nature of these CAR architectures assumes an unrealistic tumor target that has homogenous and static antigen expression; they do not capture the heterogeneity and plasticity of actual tumors. Furthermore, due to their rigid design, conventional CAR T-cells cannot be easily controlled post-infusion, and thus toxicities arising from therapy are often curbed symptomatically with anti-IL-6 targeting tocilizumab and corticosteroids (Gardner, et al., Blood 2016, 128 (22), 586). Accordingly, multi-targeting and modular CAR systems have been developed for comprehensive and adaptable cancer treatment.

For instance, universal CAR receptors that utilize adaptor-targeting, protein-based intermediates for tumor targeting, have been developed (Minutolo, et al., Frontiers in Oncology 2019, 9, 176; Liu, et al., J. of Hematology & Oncology 2019, 12 (1), 69). Instead of binding a cancer antigen directly, the universal CAR receptor recognizes a tag on a bifunctional targeting ligand, which in turn targets the cancer antigen to mediate T-cell function. By designing the cancer targeting properties extrinsic to the CAR receptor, this system addresses some weaknesses of conventional CAR architectures. For example, multiple targeting intermediates that share the same CAR-specific tag can be employed simultaneously or in-sequence depending on the dynamic characteristics of a patient's cancer, preventing antigen escape via responsive treatment. Additionally, since the targeting intermediates control antigen presentation to the CAR T-cells, the amount and frequency at which intermediates are dosed can be used to mitigate toxic side effects normally associated with therapy. For these reasons, a great number of universal CAR systems have been developed. These include systems that use natural binding partner pairs such as avidin and biotin and leucine zipper domains (Urbanska, et al., Cancer Research 2012, 72 (7), 1844-1852; Lohmueller, et al., Oncoimmunology 2018, 7 (1), e1368604; Cho, et al., Cell 2018, 173 (6), 1426-1438), as well as those that use more conventional scFvs that target small molecule or peptide tags such as FITC (Ma, et al., PNAS 2016, 113 (4), E450-E458; Lee, et al., Cancer Research 2019, 79 (2), 387-396; Lu, et al., Frontiers in Oncology 2019, 9, 151), peptide neo-epitope (PNE) derived from yeast (Rodgers, et al., PNAS 2016, 113 (4), E459-E468), and E5B9 peptide derived from human nuclear autoantigen La/SS-B (Cartellieri, et al., Blood Cancer J. 2016, 6 (8), e458).

### BRIEF DESCRIPTION OF THE DRAWINGS

Some of the drawings submitted herewith may be better understood in color. Applicants consider the color versions of the drawings as part of the original submission and reserve the right to present color images of the drawings in later proceedings.

FIG. 1 illustrates an example environment for administration of a universal CAR T-cell therapy using synthetic intermediates.

FIG. 2 illustrates components of an example universal CAR T-cell therapy using a synthetic intermediate.

FIG. 3 illustrates an example process for administering a universal CAR T-cell therapy to a subject using at least one synthetic intermediate.

FIGS. 4A-4C. SpyTag003(D2) exhibits enhanced loading onto the original SpyCatcher CAR than SpyTag. FIG. 4A shows a design of tricistronic lentiviral SpyCatcher CAR constructs with different extracellular spacer lengths. FIG. 4B shows cytometry histograms of biotinylated SpyTag loading onto H9 SpyCatcher CAR cells. Histograms are representative of 1 biological replicate. FIG. 4C illustrates flow cytometry histograms of biotinylated SpyTag003(D2) loading onto H9 SpyCatcher CAR cells. Histograms are representative of 1 biological replicate. D-arginine substitutions in the SpyTag003(D2) sequence are shown in blue. SA-AF647, streptavidin Alexa Fluor 547.

FIGS. 5A and 5B illustrate that SpyTag003(D2) displays modest proteolytic stability in serum. MALDI-ToF spectra of SpyTag003(D2) peptide incubated in (FIG. 5A) complete media spiked with BxPC3 cells and (FIG. 5B) normal mouse serum for 0, 4, 8, 12, and 24 h at 37 °C. Molecular weights of prominent peaks are shown. Predicted amino acid sequences of degradation products based on measured molecular weights are shown at the bottom of both FIGS. 5A and 5B.

FIGS. 6A-6C illustrate that cell-expressed SpyCatcher003 CARs react sensitively and quickly with modified SpyTag003 peptide. FIG. 6A (top) shows a design of tricistronic lentiviral SpyCatcher003 (DB5) CAR constructs with different extracellular spacer lengths. FIG. 6A (bottom) shows a schematic of isopeptide bond formation between lysine and aspartic acid side chains of SpyCatcher003 CAR (solid oval, green) and D-arginine substituted SpyTag003(D2) (dashed oval, purple), respectively, based on PDB 4MLI. The D-arginine residues in the SpyTag003(D2) sequence are shown. FIG. 6B shows flow cytometry loading curves of SpyTag003(D2) peptide onto H9 DB5 CAR cells with different spacers lengths, normalized to the highest observed loading. The curves represent a nonlinear regression of three independent experiments in which loading data are fitted to a Michaelis-Menten equation. FIG. 6C shows flow cytometry time kinetics of 50 nM SpyTag003(D2) loading onto H9 DB5 CAR cells with different spacer lengths, normalized to the highest observed loading. The curves represent a nonlinear regression assuming one phase association of one independent experiment. SA-AF647, streptavidin Alexa Fluor 647.

FIGS. 7A-7C illustrate that DB5 CAR-encoding lentivirus transduces immortalized T-cells with high titers. Flow cytometry plots of biotinylated Erbitux and 500 nM SpyTag003(D2) staining of H9 cells transduced with different concentrations of lentivirus encoding (FIG. 7A) short spacer, (FIG. 7B) medium spacer, and (FIG. 7C) long spacer DB5 CARs. Plots are representative of 1 biological replicate. Titers in TU/mL are listed for each lentivirus and were calculated using data from both the Erbitux and SpyTag003(D2) staining. SA-AF647, streptavidin Alexa Fluor 647.

FIGS. 8A-8C show that a branched peptide is capable of bispecific properties. FIG. 8A is a schematic representation of bifunctional C2C18(ChARK)-X-SpyTag003(D2) peptide. Cysteine substitutions and DFBP cyclization are shown in solid rectangles. Hydroxyproline and D-amino acid modifications are shown in dashed rectangles. FIG. 8B shows flow cytometry binding curves of bifunctional peptide to K562 and K562 αvβ6:mCherry cells, normalized to 400 nM binding to K562 αvβ6:mCherry cells. The curves represent a nonlinear regression of three independent experiments in which binding data are fitted to a Hill equation. K_{D} values were calculated by averaging the individual regression values of the independent experiments. Data points, error bars, and K_{D} values represent the mean±SD; n=3 independent experiments. FIG. 8C shows flow cytometry loading curves of bifunctional peptide onto H9 DB5 CAR cells with different spacers lengths, normalized to the highest observed loading. The curves represent a nonlinear regression of three independent experiments in which loading data are fitted to a Michaelis-Menten equation. Kₘ values were calculated by averaging the individual regression values of the independent experiments. Data points, error bars, and Kₘ values represent the mean±SD; n=3 independent experiments. SA-AF647, streptavidin Alexa Fluor 647.

FIG. 9 shows that CD4⁺ DB5 CAR T-cell were successfully produced within a 3-week timeline. Positively-selected CD4⁺ T-cells from a healthy donor were thawed and stimulated with activator beads on Day 0 (S₁D₀). On Day 2 (S₁D₂), 1×10⁶ cells were transduced with lentivirus encoding either short, medium, or long spacer DB5 CAR. Transduced cells were then put under 100 nM methotrexate selection on Day 4 to enrich CAR⁺ cells (S₁D₄), activator beads were removed on Day 9 (S₁D₉), and cells were stained for EGFRt transduction reporter and CAR expression on Day 11 (S₁D₁₁). Methotrexate was removed on Day 14 (S₁D₁₄) and cells were functionally characterized by an intracellular cytokine staining (ICCS) on Day 18 (S₁D₁₄). Remaining cells were frozen and banked on Day 19 (S₁D₁₉).

FIGS. 10A-10D illustrate that a bifunctional adaptor peptide directs CD4⁺ DB5 CAR T-cell cytokine responses against αvβ6⁺ target cells. FIG. 10A illustrates flow cytometry plots of biotinylated Erbitux staining and 500 nM SpyTag003(D2) loading on S₁D₁₁ CD4⁺ T-cells transduced with lentivirus encoding short, medium, and long spacer DB5 CARs. Plots are representative of 1 biological replicate. MFI values of antibody staining and peptide loading are shown. FIG. 10B shows cytometry histograms of target cell pre-labeling with 500 nM bifunctional peptide on the day of the ICCS assay. Histograms are representative of 1 biological replicate. MFI values of peptide labeling are labeled with stars. FIG. 10C illustrates flow cytometry plots of CD4⁺ DB5 CAR T-cell pre-arming with 500 nM bifunctional peptide on the day of the ICCS assay. Plots are representative of 1 biological replicate. MFI values of peptide arming are shown. FIG. 10D illustrates ICCS pie charts of IL2, TNFα, and IFNγ cytokine production in CD4⁺ DB5 CAR T-cells after 5-h co-culture with target cells. Bifunctional peptide was both pre-labeled on target cells and pre-armed on T-cells for directing DB5 CAR T-cell responses against αvβ6⁺ target cells. Pie charts are representative of 1 biological replicate.

FIGS. 11A and 11B show that K562 SpyTag003(L) cells express detectable SpyTag003 on their surface. FIG. 11A shows a design of a tricistronic lentiviral SpyTag003(L) construct. FIG. 11B shows flow cytometry plots of biotinylated Erbitux and 16.67 nM SpyCatcher Nanocage (SC50Ai Cage) staining of K562 cells transduced with SpyTag003(L) lentivirus. Unreacted SpyCatcher proteins on cage-labeled cells were stained with 1 μM SpyTag-rhodamine as a secondary stain. Surface expression of SpyTag003 was confirmed by cage staining. Plots are representative of 1 biological replicate. SA-AF647, streptavidin Alexa Fluor 647; SpyTag-Rhod, SpyTag-rhodamine.

FIG. 12 illustrates that C2C18(ChARK)-X-SpyTag003(D2) peptide is rapidly internalized by αvβ6⁺ pancreatic BxPC3 cells. Flow cytometry detection of bound C2C18(ChARK)-X-SpyTag003(D2) peptide remaining on the surface of BxPC3 cells over a 60-min incubation at 37 °C, normalized to a 0-min no incubation control. The curve represents a nonlinear regression of one independent experiment assuming one-phase exponential decay. SA-AF647, streptavidin Alexa Fluor 647.

FIG. 13 shows that CD8⁺ DB5 CAR T-cell were successfully produced within a 3-week timeline. Positively-selected CD8⁺ T-cells from a healthy donor were thawed and stimulated with activator beads on Day 0 (S₁D₀). On Day 2 (S₁D₂), 1×10⁶ cells were transduced with lentivirus encoding either short, medium, or long spacer DB5 CAR. Transduced cells were then put under 50 nM methotrexate selection on Day 4 to enrich CAR⁺ cells (S₁D₄), activator beads were removed on Day 9 (S₁D₉), and cells were stained for EGFRt transduction reporter and CAR expression on Day 11 (S₁D₁₁). Methotrexate selection was increased to 100 nM on Day 11 (S₁D₁₁) before being removed on Day 14 (S₁D₁₄). Cells were both functionally characterized by a flow-based cytoxicity assay and frozen/baked on Day 21 (S₁D₂₁).

FIGS. 14A-14C show that a bifunctional adaptor peptide directs CD8⁺ DB5 CAR T-cell cytotoxic responses against αvβ6⁺ target cells. FIG. 14A shows flow cytometry plots of biotinylated Erbitux staining and 500 nM SpyTag003(D2) loading on S₁D₁₁ CD8⁺ T-cells transduced with lentivirus encoding short, medium, and long spacer DB5 CARs. Plots are representative of 1 biological replicate. MFI values of antibody staining and peptide loading are shown. FIG. 14B shows flow cytometry plots of CD8⁺ DB5 CAR T-cell pre-arming with 500 nM bifunctional peptide on the day of the cytotoxicity assay. Plots are representative of 1 biological replicate. MFI values of peptide arming are shown. FIG. 14C shows specific lysis curves of target cells after 18-h co-culture with effector CD8⁺ DB5 CAR T-cells at 9:1, 3:1, and 1:1 E:T ratios, normalized to lysis in the absence of T-cells. Bifunctional peptide was only pre-armed on T-cells for directing DB5 CAR T-cell responses against αvβ6⁺ target cells. Curves are representative of 1 biological replicate.

FIGS. 15A to 16C show that an example aptamer-peptide chimera is capable of bispecific properties. FIG. 15A is a schematic representation of copper-free click chemistry reaction used to synthesize Aptamer-Triazole-SpyTag003(D2)-biotin chimera. DBCO and azide modifications used for the click reaction are shown in solid rectangles and dashed rectangles, respectively. FIG. 15B shows flow cytometry binding curves of aptamer-peptide chimera to K562 and JurkaT-cells. The curves represent a nonlinear regression of one independent experiments in which binding data are fitted to a Hill equation. FIG. 15C shows flow cytometry histograms of 500 nM aptamer-peptide chimera loading on CD8+DB5 CAR T-cells with different spacers lengths. Histograms are representative of two independent experiments. SA-AF647, streptavidin Alexa Fluor 647.

FIG. 16 shows that urea-PAGE confirms successful synthesis of the example aptamer-peptide chimera. SYBR Gold-stained 15% urea-PAGE gel of aptamer alone and aptamer-peptide chimera. The upward shift in the aptamer band size signifies successful conjugation of peptide onto the aptamer via copper-free click chemistry.

FIG. 17 illustrates a protein sequence alignment of a N-terminal truncated SpyCatcher (SpyCatcherΔN) and SpyCatcher003 using the Clustal Omega program. Solid boxes represents mutations made from SpyCatcher to SpyCatcher002. Dashed boxes represent mutations made from SpyCatcher002 to SpyCatcher003. An N-terminal truncated SpyCatcher003 would retain all the mutations made to SpyCatcher002 for increased reaction kinetics.

FIG. 18 illustrates peptide sequences used in a first Experimental Example.

FIG. 19A illustrates results of the bifunctional folate peptide chimera bonding to FOLR1 protein, as measured by biolayer interferometry. FIG. 19B illustrates a fluorescence activated cell sorting analysis of b-SpyTag003(2D)-K (folate) binding to FOLR+KB cells in vitro. FIGS. 19C to 19E illustrate DB5 CAR T-cell responses with a bifunctional chimeric small molecule peptide-adapter.

FIG. 20 illustrates loading kinetics for SpyTag003(2D) peptides, which may have increased serum stability.

FIG. 21A illustrates the experimental protocol followed in the Fourth Experimental Example. FIG. 21B illustrates corresponding tumor volume measurements over 23-day study comparing left (K562) and right (K562 αvβ6:mCherry) tumors in each group.

FIG. 22 illustrates various sequences referenced in the present disclosure.

### DETAILED DESCRIPTION

While previously developed universal CAR systems are powerful, they may be limited by noncovalent recognition of the tag-labeled targeting intermediates. Conventional CARs rely on a single affinity-based interaction for activation, whereas universal CAR systems required two such interactions to occur in sequence. Thus, in various cases, CAR binding of the tag-labeled ligand and ligand binding to the cancer antigen should synchronize, and the transient properties (i.e., off-rate) of any one event can cause the entire system to fail.

Recognizing the slippery nature of antibody binding to peptide-based tags, Zakeri et al. developed SpyCatcher and SpyTag, a protein and peptide pair split from the collagen adhesion domain from Streptococcus pyogenes and rationally optimized them to form a covalent isopeptide with each other (Zakeri, et al., PNAS 2012, 109 (12), E690-E697). The spontaneous reaction between SpyCatcher and SpyTag occurs under diverse conditions (pH 5-10, 4-37 °C), and their ability to be genetically encoded and fused to other proteins at either their N-terminus or C-terminus without loss of function has proven them to be valuable tools for various biological applications (see, e.g., Sun, et al., PNAS 2014, 111(31), 11269-11274; Reddington, et al., Current Opinion in Chemical Biology 2015, 29, 94-99; Brune, et al., Scientific Reports 2016, 6(1), 19234). Given these favorable attributes, this covalent technology has recently been adopted into universal CAR platforms with SpyCatcher-modified CARs and SpyTag-labeled targeting intermediates such as scFvs, designed ankyrin repeat proteins (DARPins), and antibodies (Liu, et al., Therapeutic Advances in Medical Oncology 2020, 12, 1-12; Minutolo, et al., J. of the American Chemical Society 2020, 142 (14), 6554-6568).

SpyCatcher and SpyTag, however, have slow reaction kinetics that utilize micromolar concentrations of both partners for efficient covalent bond formation (Zakeri, et al., PNAS 2012, 109 (12), E690-E697). Consequentially, previously developed universal CAR systems using this pair lose sensitivity if target antigen expression or intermediate ligand concentration is low (Minutolo, et al., J. of the American Chemical Society 2020, 142 (14), 6554-6568).

Various implementations of the present disclosure relate to synthetic intermediates (also referred to as “intermediaries”) for universal CAR T therapies. Herein, “synthetic” refers to both fully and partially synthetic molecules. For example, a synthetic intermediate includes intermediaries that are biologically produced and conjugated to a synthetic targeting ligand. In particular implementations, a synthetic intermediate includes an intermediate that is fully synthetic.

Various synthetic intermediates described herein have relatively small sizes (e.g., masses) and present enhanced reaction kinetics, compared to previously developed, protein-based intermediates. Accordingly, therapeutically effective doses of the synthetic intermediates describe herein may be significantly lower than therapeutically effective doses of the protein-based intermediates that have been previously described. Moreover, synthetic intermediates described herein can penetrate dense tissues, enabling universal CAR T-cell therapies that target cancer cells present within solid tumors. In some examples, the enhanced reaction kinetics of synthetic intermediates also enable therapeutically effective subcutaneous administrations of the synthetic intermediates, in addition to intravenous administrations.

Particular implementations of the present disclosure also include CARs and intermediates that express novel binding domains. For instance, an example intermediate may include the novel binding domain that specifically binds to a universal CAR. Examples of the novel binding domain are set forth in SEQ ID NO: 15, SEQ ID NO: 31, SEQ ID NO: 32, SEQ ID NO: 33, SEQ ID NO: 34, or SEQ ID NO: 35, but implementations are not limited to these specific sequences.

Implementations of the present disclosure will now be described with reference to the accompanying figures.

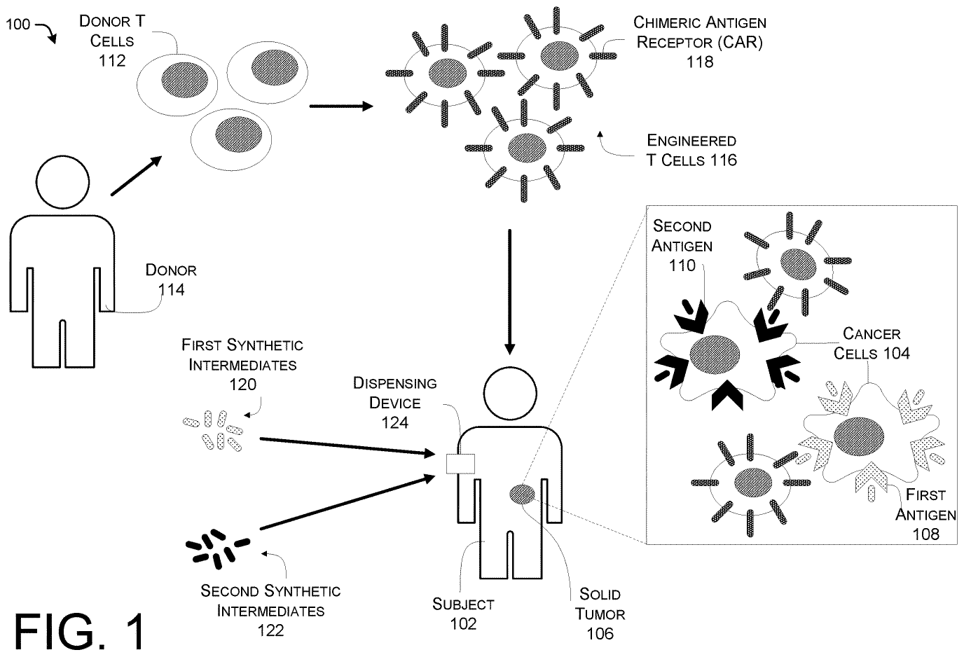

FIG. 1 illustrates an example environment 100 for administration of a universal CAR T-cell therapy using synthetic intermediates. In this example, a subject 102 suffers from a pathology due to the presence of undesirable cells inside of the body of the subject 102. The subject 102, for instance, is a human or other animal. In some cases, the subject 102 has at least one type of cancer, such as adrenal cancer, bladder cancer, blood cancer, bone cancer, brain cancer, breast cancer, carcinoma, cervical cancer, colon cancer, colorectal cancer, corpus uterine cancer, ear, nose and throat (ENT) cancer, endometrial cancer, esophageal cancer, gastrointestinal cancer, head and neck cancer, Hodgkin's disease, intestinal cancer, kidney cancer, larynx cancer, leukemia, liver cancer, lymph node cancer, lymphoma, lung cancer, melanoma, mesothelioma, myeloma, nasopharynx cancer, a neuroblastoma, non-Hodgkin's lymphoma, oral cancer, ovarian cancer, pancreatic cancer, penile cancer, pharynx cancer, prostate cancer, rectal cancer, sarcoma, seminoma, skin cancer, stomach cancer, a teratoma, testicular cancer, thyroid cancer, uterine cancer, vaginal cancer, a vascular tumor, or combinations or metastases thereof. In some examples, the subject 102 has In some embodiments, the subject 102 has a B cell cancer (multiple myeloma), a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. For instance, cancer cells 104 are present in the body of the subject 102. The cancer cells 104 are abnormal cells that divide uncontrollably in the body of the subject 102. The cancer cells 104 in the subject 102 form a solid tumor 106 in the body of the subject 102. As used herein, the terms “tumor,” “neoplasm,” and their equivalents, may refer to a mass of tissue including cancer cells.

The cancer cells 104 express a first antigen 108 and a second antigen 110. In various cases, the first antigen 108 and the second antigen 110 are expressed on the surfaces of the cancer cells 104. For instance, the first antigen 108 and the second antigen 110 may include proteins, peptides, polysaccharides, lipids, nucleic acid molecules, or any combination thereof.

Although FIG. 1 illustrates an example in which the first antigen 108 and the second antigen 110 are cancer antigens, implementations of the present disclosure are not so limited. For example, implementations of the present disclosure can be used to target cells that express a viral antigen or some other type of antigen. In some cases, implementations can be used to target cells expressed by antigens associated with autoimmune disorders (e.g., GAD65, citrullinated vimentin, myelin oligodendrocyte glycoprotein) or heart disease (fibroblast activation protein). In some cases, the first antigen 108 and the second antigen 110 are different antigens. Examples of the first antigen 108 and/or the second antigen 110, for instance, include one or more of DLL3, CLL1, GPRC5D, EpCAM, CD3, CD5, CD7, fibroblast activation protein, GAD65, citrullinated vimentin, myelin oligodendrocyte glycoprotein, carcinoembryonic antigen, prostate specific antigen, Prostate Stem Cell antigen, PSMA, Her2/neu, estrogen receptor, progesterone receptor, ephrinB2, CD19, CD20, CD22, CD23, CD123, CS-1, CE7, hB7H3, ROR1, mesothelin, c-Met, GD-2, EGFR, EGFRvIII, EphA2, IL13Ra2, L1CAM, oaGD2, GD2, B7H3, CD33, VAR2CSA, MUC16, PD-L1, ERBB2, folate receptor, biotin receptor, CD56; glypican-2, disialoganglioside, EpCam, L1-CAM, Lewis Y, WT-1, Tyrosinase related protein 1, GD2, B-cell maturation antigen, CD24, SV40 T, carboxy-anhydrase-IX, or CD13.

In various examples, at least one care provider may diagnose the subject 102 with cancer. For example, a clinician (e.g., a pathologist) may perform histological staining and analysis on a tissue sample from the solid tumor 106 in order to identify the presence of the cancer cells 104. In some cases, a clinician (e.g., a radiologist) may identify the solid tumor 106 using medical imaging techniques, such as PET or SPECT.

In particular cases, the care provider(s) determine that the cancer cells 104 express the first antigen 108 and the second antigen 110. For example, a clinician (e.g., a pathologist) may perform immunohistological staining and analysis on the tissue sample in order to determine that the cancer cells 104 express the first antigen 108 and the second antigen 110 on the surfaces of the cancer cells 104. In some cases, a clinician may cause a radiolabel to attach to the first antigen 108 and the second antigen 110 and may determine that the cancer cells 104 express the first antigen 108 and the second antigen 110 by detecting the presence of the radiolabel in a PET scan of the subject 102.

The first antigen 108 and the second antigen 110, in various cases, are preferentially expressed by the cancer cells 104 in the body of the subject 102. For instance, the first antigen 108 and the second antigen 110 are not expressed, or are minimally expressed, by other cells in the body of the subject 102. Accordingly, a therapy designed to kill cells that express the first antigen 108 and the second antigen 110 is a specific therapy that targets the cancer cells 104.

In various examples, the cancer cells 104 of the subject 102 are targeted with a CAR T-cell therapy. The term “CAR T-cell therapy,” and its equivalents, may refer to a treatment in which immune cells, such as T-cells, are genetically engineered to express a CAR that enables the immune cells to identify and kill cells expressing a predetermined target. For example, a CAR T-cell therapy can be used to specifically destroy cancer cells.

Donor T-cells 112 are obtained from a donor 114. In various cases, the donor 114 is not the subject 102. Although not specifically illustrated in FIG. 1, in some examples, the donor 114 is the subject 102.

In various cases, engineered T-cells 116 are generated based on the donor T-cells 112. For instance, the engineered T-cells 116 are engineered to express a CAR 118. In various cases, the CAR 118 includes a sequence that has at least 60%, 70%, 80%, 90%, 95%, 98%, or 99% sequence identity to SEQ ID. NO: 1, SEQ ID NO: 2, SEQ ID NO. 3, SEQ ID NO: 21, SEQ ID NO: 22, SEQ ID NO: 29, or SEQ ID NO: 30.

Although FIG. 1 illustrates an example utilizing donor T-cells 112 and engineered T-cells 116, implementations are not so limited. Various types of immune cells can be substituted for the donor T-cells 112 and the engineered T-cells 116, such as natural killer (NK) cells, macrophages, NK T-cells (NKT) cells, induced pluripotent stem cell (iPSC)-derived cells, or hematopoietic stem cells.

In a conventional CAR T-cell therapy, the CAR 118 could include a binding domain designed to directly bind the first antigen 108 or the second antigen 110. The engineered T-cells 116 would be administered to the subject 102. While in the body of the subject 102, the engineered T-cells 116 bind the targeted antigen using the CAR 118 and kill the cancer cells 104 that express the targeted antigen on their surfaces.

However, conventional CAR T-cell therapy has a number of significant drawbacks. For instance, the substantial efforts to generate the engineered T-cells 116 make it expensive and difficult to generate the engineered T-cells 116 to express different types of CARs (e.g., other than the CAR 118). Conventional CAR T-cell therapies therefore typically target a single type of antigen (e.g., either the first antigen 108 or the second antigen 110), rather than multiple antigens (e.g., both the first antigen 108 and the second antigen 110). Accordingly, conventional CAR T-cell therapies are unable to target heterogeneous cancers that include multiple types of cancer cells 104.

A further limitation of conventional CAR T-cell therapy relates to immune response of the subject 102. In some cases, administration of the engineered T-cells 116 can cause a severe immune reaction of the subject 102. For example, in some cases, where the CAR 118 binds to an antigen directly on cancer cells 104 in the subject 102, the administration of the engineered T-cells 116 may cause CRS and/or neurotoxicity. Because the engineered T-cells 116 persist in the body of the subject 102 for an extended period of time, the only feasible means of addressing the immune response is to treat the symptoms of the immune response, such as through the administration of anti-IL-6 targeting tocilizumab and corticosteroids. There are limited options for reducing the amount of engineered T-cells 116 causing the immune response, for instance.

Universal CAR T-cell therapy is an alternative to conventional CAR T-cell therapy, and addresses many of these problems. The terms “universal CAR T-cell therapy,” “UCAR T-cell therapy,” and their equivalents, may refer to a treatment in which a CAR, as expressed by an engineered T-cell, is designed to specifically bind an intermediate, and the intermediate is designed to specifically bind an antigen expressed by a targeted cell. When the intermediate binds the antigen, and the CAR expressed by the engineered T-cell binds the intermediate, the engineered T-cell may kill the targeted cell expressing the antigen. The term “intermediate” may refer to a construct including a first binding domain that specifically binds a CAR and a second binding domain that specifically binds an antigen. In some examples, an intermediate may be a protein (e.g., an antibody) or other biological macromolecule.

Large intermediates, such as proteins, have some limitations. For example, protein-based intermediates may have undesirable kinetics when injected into the body of the subject 102. Due to the substantial size of protein-based intermediates, they are unable to penetrate dense tissues such as the solid tumor 106. Thus, the portion of the cancer cells 104 located deep within the solid tumor 106 may not be efficiently targeted by a universal CAR T-cell therapy utilizing protein-based intermediates.

Further, in some cases, large intermediates are administered invasively. For example, a protein-based intermediate may circulate in the body of the subject 102 after being administered intravenously, but would not circulate in the body of the subject 102 if administered subcutaneously. Thus, the administration of protein-based intermediates may be uncomfortable to the subject 102.

In various implementations of the present disclosure, synthetic intermediates are used for a universal CAR T-cell therapy. For example, the CAR 118 expressed by the engineered T-cells 116 specifically binds at least two types of intermediates: first synthetic intermediates 120 and second synthetic intermediates 122. The term “synthetic intermediate” may refer to an intermediate for universal CAR T-cell therapy that is composed of at least one synthetic molecule. In various cases, a synthetic intermediate is smaller than a protein, such as an antibody. Various synthetic intermediates described herein may have masses in a range of 0.5 kDa to 200 kDa. Some examples of synthetic intermediates that include a co-polymer formulation may have masses in a range of 0.5 kDa to 200 kDa. For instance, the first synthetic intermediates 120 and the second synthetic intermediates 122 may include peptides (branched and/or unbranched), DNA aptamers, small molecules (e.g., molecules having a mass of less than 1 kDa), peptide-DNA aptamer chimeras, small molecule-peptide chimeras, or co-polymer formulations.

According to various examples, the first synthetic intermediates 120 and the second synthetic intermediates 122 each include a tag. The term “tag,” and its equivalents, may refer to any sequence of monomers (e.g., nucleotide bases, amino acids, or the like, which may or may not be naturally occurring) that specifically binds a CAR. For instance, the tag in the first synthetic intermediates 120 and/or the second synthetic intermediates 122 may include a sequence that has at least 60%, 70%, 80%, 90%, 95%, 98%, or 99% sequence identity to SEQ ID NO: 5, SEQ ID NO: 6, SEQ ID NO: 14, SEQ ID NO: 15, SEQ ID NO: 20, SEQ ID NO: 23, SEQ ID NO: 31, SEQ ID NO: 32, SEQ ID NO: 33, SEQ ID NO: 34, SEQ ID NO: 35, SEQ ID NO: 36, or SEQ ID NO: 37.

According to various cases, the CAR 118 includes an intermediary-binding domain that specifically binds the tag in the first synthetic intermediates 120 and the second synthetic intermediates 122. For instance, the intermediary-binding domain has at least 60%, 70%, 80%, 90%, 95%, 98%, or 99% sequence identity to SEQ ID NO: 5, SEQ ID NO: 6, SEQ ID NO: 14, SEQ ID NO: 20, SEQ ID NO: 23, or SEQ ID NO: 37.

The first synthetic intermediates 120 include a first antigen-binding domain that specifically binds the first antigen 108. The second synthetic intermediates 122 include a second antigen binding domain that specifically binds the second antigen 110. In some implementations, an individual instance of first synthetic intermediates 120 includes multiple copies of the first antigen-binding domain, such that the instance of the first synthetic intermediates 120 may bind multiple instances of the first antigen (e.g., multiple cells expressing the first antigen). In some implementations, an individual instance of second synthetic intermediates 122 includes multiple copies of the second antigen-binding domain, such that the instance of the second synthetic intermediates 122 may bind multiple instances of the second antigen. According to various implementations, instances of the first synthetic intermediates 120 and/or the second synthetic intermediates 122 may include multiple copies of the tag, such that they can simultaneously bind multiple instances of the CAR 118.

According to some examples, the relatively small sizes of the first synthetic intermediates 120 and the second synthetic intermediates 122 provide some advantages over non-synthetic intermediates (e.g., antibody intermediates). For instance, the first synthetic intermediates 120 and the second synthetic intermediates 122 may reach the cancer cells 104 in the solid tumor 106 after being administered to the subject 102 subcutaneously. In some examples, the first synthetic intermediates 120 and the second synthetic intermediates 122 are loaded into a dispensing device 124 that is disposed on the skin of the subject 102. The dispensing device 124, for instance, is configured to subcutaneously administer dosages of the first synthetic intermediates 120 and the second synthetic intermediates 122 to the subject 102. Accordingly, dosages of the first synthetic intermediates 120 and the second synthetic intermediates 122 may be administered to the subject 102 less invasively, and with less discomfort, than protein-based intermediates that are administered intravenously. Notably, in some implementations of the present disclosure, the first synthetic intermediates 120 and the second synthetic intermediates 122 can also be administered intravenously. In some examples, the first synthetic intermediates 120 and the second synthetic intermediates 122 can be administered in an oral formulation that is orally consumed by the subject 102 (e.g., using techniques described in Drucker, Nat Rev Drug Discov 19, 277-289 (2020).

Further, an effective dosage of the first synthetic intermediates 120 and the second synthetic intermediates 122 may be lower than an effective dosage of otherwise equivalent, but protein-based intermediates. In some cases, the first synthetic intermediates 120 and the second intermediates 122 are effective at nanomolar concentrations in the vicinity of the cancer cells 104.

In some cases, the small sizes of the first synthetic intermediates 120 and the second synthetic intermediates 122 enable them to penetrate the solid tumor 106 via enhanced kinetics. Accordingly, cancer cells 104 located deep within the solid tumor 106 may be bound by the first synthetic intermediates 120 and the second synthetic intermediates 122. This increased binding can lead to enhanced targeting by the engineered T-cells 116, such that the first synthetic intermediates 120 and the second synthetic intermediates 122 enable universal CAR T-cell therapies to target cancer cells 104 disposed below the surface of blood vessels in the solid tumor 106.

According to some examples, the first synthetic intermediates 120 and the second synthetic intermediates 122 can be further used to provide a treatment regimen for the subject 102 that is adaptive to a response by the subject 102 to the engineered T-cells 116, the first synthetic intermediates 120, the second synthetic intermediates 122, and other triggers for changes in the condition of the subject 102. In various cases, the first synthetic intermediates 120 and the second synthetic intermediates 122 are removed from the body of the subject 102 over time, such as by the renal system of the subject 102. Thus, a concentration of the first synthetic intermediates 120 and the second synthetic intermediates 122 within the body of the subject 102 may decrease over time after administration. In some cases, without administration of additional dosages, the first synthetic intermediates 120 and second synthetic intermediates 122 may decrease to minimal concentration within the body of the subject 102 over the course of minutes, hours, or days.

In some implementations, the dispensing device 124 is configured to administer dosages of the first synthetic intermediates 120 and the second synthetic intermediates 122 based on a response of the subject 102 to the engineered T-cells 116, the first synthetic intermediates 120, and the second synthetic intermediates 122. For instance, if the subject 102 has an intolerable response to an initial dosage of the first synthetic intermediates 120, the dispensing device 124 may subsequently administer a lower dosage of the first synthetic intermediates 120. In some cases, if the subject 102 tolerates the initial dosage of the first synthetic intermediates 120, the dispensing device 124 subsequently administers the same or a higher dosage of the first synthetic intermediates 120 to the subject 102.

According to some cases, the dispensing device 124 is configured to time the administration of multiple dosages of the first synthetic intermediates and the second synthetic intermediates 122 based on the response of the subject 102 to the engineered T-cells 116, the first synthetic intermediates 120, and the second synthetic intermediates 122. In some examples, if the subject 102 has a negative immune response to an initial dosage of the second synthetic intermediates 122, the dispensing device 124 may lengthen a time period until administration of a subsequent dosage of the second synthetic intermediates 122. In cases where the subject 102 has a limited or no immune response to the initial dosage of the second synthetic intermediates 122, the dispensing device 124 may shorten a time period until administration of a subsequent dosage of the second synthetic intermediates 122. By controlling the amount and timing of dosages of the first synthetic intermediates 120 and/or the second synthetic intermediates 122, the cancer cells 104 in the body of the subject 102 can be selectively destroyed by the engineered T-cells 116 without inducing a significant immune response in the subject 102.

In some examples, the dispensing device 124 may further output an inhibitory construct that includes the tag without an antigen binding-domain, in order to reduce a side effect of administration of the engineered T-cells 116. For example, in response to determining that the subject 102 has an intolerable response (e.g., CRS) to the engineered T-cells 116, the dispensing device 124 may administer a formulation including the inhibitory construct. The inhibitory construct may bind the CAR 118 expressed by the engineered T-cells 116, thereby preventing the engineered T-cells 116 from binding other cells or other elements in the body of the subject 102. The inhibitory constructs, for instance, may include at least one of proteins, peptides, aptamers, peptide-aptamer chimeras, small molecules, small-molecule-peptide chimeras, co-polymer formulations, or the like.

In some implementations, the universal CAR T-cell therapy can be adapted based on changing conditions of the subject 102, the cancer cells 104, the solid tumor 106, or any combination thereof. In some cases, expression of the cancer cells 104 in the body of the subject 102 may change over time. For example, after initial dosages of the first synthetic intermediates 120, the cancer cells 104 expressing the first antigen 108 may be eliminated from the body of the subject 102. However, at least some of the cancer cells 104 may express a third antigen (not illustrated) without expressing the first antigen 108 or the second antigen 110. In some cases, third synthetic intermediates that bind the third antigen can be manufactured and administered to the subject 102 after the initial dosages of the first synthetic intermediates 120. These third synthetic intermediates, for instance, are significantly easier to manufacture than the engineered T-cells 116. Therefore, in some cases, the therapy administered to the subject 102 can be revised based on changing expression of the cancer cells 104.

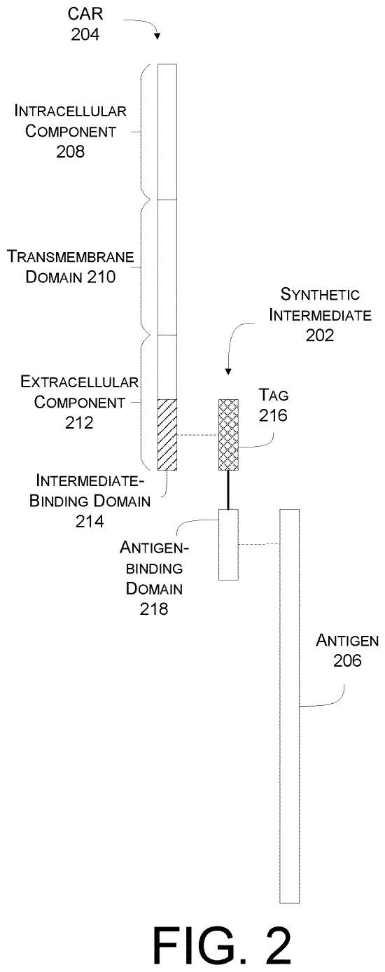

FIG. 2 illustrates components of an example universal CAR T-cell therapy using a synthetic intermediate 202. In various implementations, the synthetic intermediate 202 binds to a CAR 204 and an antigen 206. The CAR 204, for instance, is expressed by a cell, such as an immune cell.

The CAR 204, for instance, includes an intracellular component 208, a transmembrane domain 210, and an extracellular component 212. In various cases, when the CAR 204 is expressed by a cell (e.g., a lymphocyte), the intracellular component 208 is disposed inside of the cell, the transmembrane domain 210 is at least partially disposed through the cell membrane of the cell, and the extracellular component 212 protrudes from a surface of the cell. In some implementations, the CAR 204 has at least 60%, 70%, 80%, 90%, 95%, 98%, or 99% sequence identity to SEQ ID NO: 1, SEQ ID NO: 2, SEQ ID NO: 3, SEQ ID NO: 21, SEQ ID NO: 22, SEQ ID NO: 29, or SEQ ID NO: 30. In some cases, the CAR 204 includes a cleavable linker. For example, the cleavable linker includes at least one of P2A, T2A, E2A, or F2A. In some cases, the cleavable linker includes a sequence having at least 60%, 70%, 80%, 90%, 95%, 98%, or 99% sequence identity to SEQ ID NO: 24, SEQ ID NO: 25, SEQ ID NO: 26, or SEQ ID NO: 27, SEQ ID NO: 28.

In some cases, the intracellular component 208 includes an effector domain that is responsible for activation of the cell in which the CAR 204 is expressed. The term “effector domain” is thus meant to include any portion of the intracellular domain sufficient to transduce an activation signal. An effector domain can directly or indirectly promote a biological or physiological response in a cell when receiving the appropriate signal. In certain implementations, an effector domain is part of a protein or protein complex that receives a signal when bound, or it binds directly to a target molecule, which triggers a signal from the effector domain. An effector domain may directly promote a cellular response when it contains one or more signaling domains or motifs, such as an immunoreceptor tyrosine-based activation motif (ITAM). In other implementations, an effector domain will indirectly promote a cellular response by associating with one or more other proteins that directly promote a cellular response, such as co-stimulatory domains.

Effector domains can provide for activation of at least one function of a modified cell upon binding to the cellular marker expressed by a cancer cell. Activation of the modified cell can include one or more of differentiation, proliferation and/or activation or other effector functions. In particular implementations, an effector domain can include an intracellular signaling component including a T-cell receptor and a co-stimulatory domain which can include the cytoplasmic sequence from co-receptor or co-stimulatory molecule.

An effector domain can include one, two, three, or more intracellular signaling components (e.g., receptor signaling domains, cytoplasmic signaling sequences), co-stimulatory domains, or combinations thereof. Exemplary effector domains include signaling and stimulatory domains selected from: 4-1BB (CD137), CARD11, CD3γ, CD3δ, CD3ε, CD3ζ, CD27, CD28, CD79A, CD79B, DAP10, FcRα, FcRβ (FcεR1b), FcRγ, Fyn, HVEM (LIGHTR), ICOS, LAG3, LAT, Lck, LRP, NKG2D, NOTCH1, pTα, PTCH2, OX40, ROR2, Ryk, SLAMF1, Slp76, TCRα, TCRβ, TRIM, Wnt, Zap70, or any combination thereof. In particular implementations, exemplary effector domains include signaling and co-stimulatory domains selected from: CD86, FcγRIIa, DAP12, CD30, CD40, PD-1, lymphocyte function-associated antigen-1 (LFA-1), CD2, CD7, LIGHT, NKG2C, B7-H3, a ligand that specifically binds with CD83, CDS, ICAM-1, GITR, BAFFR, SLAMF7, NKp80 (KLRF1), CD127, CD160, CD19, CD4, CD8α, CD8β, IL2Rβ, IL2Rγ, IL7Rα, ITGA4, VLA1, CD49a, IA4, CD49D, ITGA6, VLA-6, CD49f, ITGAD, CD11d, ITGAE, CD103, ITGAL, CD11a, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), PSGL1, CD100 (SEMA4D), CD69, SLAMF6 (NTB-A, Ly108), SLAM (CD150, IPO-3), BLAME (SLAMF8), SELPLG (CD162), LTBR, GADS, PAG/Cbp, NKp44, NKp30, NKp46, TLR1, TLR2, TLR3, TLR4, TLR5, TLR6, TLR7, TLR8, or TLR9. In particular implementations, the effector domain includes a CD3ζ signaling domain.

Intracellular signaling component sequences that act in a stimulatory manner may include iTAMs. Examples of iTAMs including primary cytoplasmic signaling sequences include those derived from CD3γ, CD3δ, CD3ε, CD3ζ, CD5, CD22, CD66d, CD79a, CD79b, and common FcRγ (FCER1G), FcγRIIa, FcRβ (FcεRib), DAP10, and DAP12. In particular implementations, variants of CD3ζ retain at least one, two, three, or all ITAM regions.

In particular implementations, an effector domain includes a cytoplasmic portion that associates with a cytoplasmic signaling protein, wherein the cytoplasmic signaling protein is a lymphocyte receptor or signaling domain thereof, a protein including a plurality of ITAMs, a co-stimulatory domain, or any combination thereof.

Additional examples of intracellular signaling components include the cytoplasmic sequences of the CD3ζ chain, and/or co-receptors that act in concert to initiate signal transduction following binding domain engagement.

A co-stimulatory domain is a domain whose activation can be required for an efficient lymphocyte response to cellular marker binding. Some molecules are interchangeable as intracellular signaling components or co-stimulatory domains. Examples of costimulatory domains include CD27, CD28, 4-1BB (CD 137), OX40, CD30, CD40, PD-1, ICOS, lymphocyte function-associated antigen-1 (LFA-1), CD2, CD7, LIGHT, NKG2C, B7-H3, and a ligand that specifically binds with CD83. For example, CD27 co-stimulation has been demonstrated to enhance expansion, effector function, and survival of human CART-cells in vitro and augments human T-cell persistence and anti-cancer activity in vivo (Song et al. Blood. 2012; 119(3): 696-706). Further examples of such co-stimulatory domain molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CDIId, ITGAE, CD103, ITGAL, CDIIa, ITGAM, CDI Ib, ITGAX, CDIIc, ITGBI, CD29, ITGB2, CD18, ITGB7, TNFR2, TRANCE/RANKL, DNAM1 (CD226), SLAMF4 (CD244, 2B4), CD84, CD96 (Tactile), NKG2D, CEACAM1, CRTAM, Ly9 (CD229), PSGL1, CD100 (SEMA4D), CD69, SLAMF6 (NTB-A, LyI08), SLAM (SLAMF1, CD150, IPO-3), BLAME (SLAMF8), SELPLG (CD162), LTBR, LAT, GADS, SLP-76, PAG/Cbp, and CD19a. In particular implementations, the co-stimulatory domain includes a 4-1BB signaling domain. In particular implementations, the intracellular component 208 includes (i) all or a portion of the signaling domain of CD3ζ, (ii) all or a portion of the signaling domain of 4-1BB, or (iii) all or a portion of the signaling domain of CD3ζ and 4-1BB. The intracellular signaling domain, in some cases, includes at least one of aCD3ζ intracellular signaling domain, a 4-1BB intracellular signaling domain, or a CD28gg intracellular signaling domain. In some cases, the intracellular signaling domain includes a sequence having at least 60%, 70%, 80%, 90%, 95%, 98%, or 99% sequence identity to SEQ ID NO: 11, SEQ ID NO: 12, or SEQ ID NO: 13. For example, the intracellular component 208 includes a sequence having at least 60%, 70%, 80%, 90%, 95%, 98%, or 99% sequence identity to SEQ ID NO: 11, SEQ ID NO: 12, or SEQ ID NO: 13.

The intracellular component 208 may also include one or more of a protein of a Wnt signaling pathway (e.g., LRP, Ryk, or ROR2), NOTCH signaling pathway (e.g., NOTCH1, NOTCH2, NOTCH3, or NOTCH4), Hedgehog signaling pathway (e.g., PTCH or SMO), receptor tyrosine kinases (RTKs) (e.g., epidermal growth factor (EGF) receptor family, fibroblast growth factor (FGF) receptor family, hepatocyte growth factor (HGF) receptor family, insulin receptor (IR) family, platelet-derived growth factor (PDGF) receptor family, vascular endothelial growth factor (VEGF) receptor family, tropomycin receptor kinase (Trk) receptor family, ephrin (Eph) receptor family, AXL receptor family, leukocyte tyrosine kinase (LTK) receptor family, tyrosine kinase with immunoglobulin-like and EGF-like domains 1 (TIE) receptor family, receptor tyrosine kinase-like orphan (ROR) receptor family, discoidin domain (DDR) receptor family, rearranged during transfection (RET) receptor family, tyrosine-protein kinase-like (PTK7) receptor family, related to receptor tyrosine kinase (RYK) receptor family, or muscle specific kinase (MuSK) receptor family); G-protein-coupled receptors, GPCRs (Frizzled or Smoothened); serine/threonine kinase receptors (BMPR or TGFR); or cytokine receptors (IL1R, IL2R, IL7R, or IL15R). According to some cases, the intracellular component 208 includes a selection marker and/or a transduction marker.

In various cases, the transmembrane domain 210 within the CAR 204 serves to connect the extracellular component 212 and intracellular component 208 through the cell membrane of the host cell (e.g., the immune cell expressing the CAR 204). The transmembrane domain 210 can anchor the expressed molecule in the modified cell's membrane.

The transmembrane domain 210 can be derived either from a natural and/or a synthetic source. When the source is natural, the transmembrane domain 210 can be derived from any membrane-bound or transmembrane protein. Transmembrane domains can include at least the transmembrane region(s) of the α, β or ζ chain of a T-cell receptor, CD28, CD27, CD3 epsilon, CD45, CD4, CD5, CD8, CD9, CD16, CD22; CD33, CD37, CD64, CD80, CD86, CD134, CD137 CD154, Toll-like receptor 1 (TLR1), TLR2, TLR3, TLR4, TLR5, TLR6, TLR7, TLR8, and TLR9. In particular implementations, the transmembrane domain 210 may include at least the transmembrane region(s) of, e.g., KIRDS2, OX40, CD2, CD27, LFA-1 (CD 11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2Rβ, IL2Rγ, IL7R a, ITGA1, VLA1, CD49a, ITGA4, IA4, CD49D, ITGA6, VLA-6, CD49f, ITGAD, CDI Id, ITGAE, CD103, ITGAL, CDI Ia, ITGAM, CDI Ib, ITGAX, CDI Ic, ITGB1, CD29, ITGB2, CD18, ITGB7, TNFR2, DNAM1 (CD226), SLAMF4 (CD244, 2B4), CD84, CD96 (Tactile), CEACAM1, CRT AM, Ly9 (CD229), PSGL1, CD100 (SEMA4D), SLAMF6 (NTB-A, LyI08), SLAM (SLAMF1, CD150, IPO-3), BLAME (SLAMF8), SELPLG (CD162), LTBR, PAG/Cbp, NKG2D, or NKG2C. In particular implementations, a variety of human hinges can be employed as well including the human Ig (immunoglobulin) hinge (e.g., an IgG4 hinge, an IgD hinge), a GS linker (e.g., a GS linker described herein), a KIR2DS2 hinge or a CD8a hinge. In particular implementations, the transmembrane domain 210 includes a CD28 transmembrane domain. The transmembrane domain 210, for instance, includes a sequence having at least 60%, 70%, 80%, 90%, 95%, 98%, or 99% sequence identity to SEQ ID NO: 10.

In particular implementations, the transmembrane domain 210 has a three-dimensional structure that is thermodynamically stable in a cell membrane, and generally ranges in length from 15 to 30 amino acids. The structure of the transmembrane domain 210 can include an α helix, a β barrel, a β sheet, a β helix, or any combination thereof.

The transmembrane domain 210 can include one or more additional amino acids adjacent to the transmembrane region, e.g., one or more amino acid within the extracellular region of the CAR (e.g., up to 15 amino acids of the extracellular region) and/or one or more additional amino acids within the intracellular region of the CAR (e.g., up to 15 amino acids of the intracellular components). In one aspect, the transmembrane domain 210 is from the same protein that the signaling domain, co-stimulatory domain, or the hinge domain is derived from. In another aspect, the transmembrane domain 210 is not derived from the same protein that any other domain of the CAR 204 is derived from. In some instances, the transmembrane domain 210 can be selected or modified by amino acid substitution to avoid binding of such domains to the transmembrane domains of the same or different surface membrane proteins to minimize interactions with other unintended members of the receptor complex.

The extracellular component 212, in various cases, includes an intermediate binding domain 214. The intermediate binding domain 214, for instance, includes a sequence having at least 60%, 70%, 80%, 90%, 95%, 98%, or 99% sequence identity to SEQ ID NO: 5, SEQ ID NO: 6, SEQ ID NO: 14, SEQ ID NO: 20, SEQ ID NO: 23, or SEQ ID NO: 31.

According to some cases, the extracellular component 212 further includes a spacer located between the transmembrane domain 210 and the intermediate binding domain 214. For example, the inclusion of a spacer and/or one or more linker sequences can allow the CAR 204 to have additional conformational flexibility, often increasing the ability of the intermediate binding domain 214 to bind the synthetic intermediate 202.

Spacers are used to create appropriate distances and/or flexibility from other CAR sub-components. As indicated, in particular implementations, the length of a spacer is customized for binding targeted cells and mediating destruction. In particular implementations, a spacer length can be selected based upon the location of a cellular marker epitope, affinity of a binding domain for the epitope, and/or the ability of the binding agent to mediate cell destruction following binding.

Spacers can include those having 10 to 250 amino acids, 10 to 200 amino acids, 10 to 150 amino acids, 10 to 100 amino acids, 10 to 50 amino acids, or 10 to 25 amino acids.

Spacers can include those having 10 to 250 amino acids, 10 to 200 amino acids, 10 to 150 amino acids, 10 to 100 amino acids, 10 to 50 amino acids, or 10 to 25 amino acids.

In particular implementations, a spacer is 10 amino acids, 12 amino acids, 14 amino acids, 20 amino acids, 21 amino acids, 26 amino acids, 27 amino acids, 45 amino acids, 50 amino acids, 55 amino acids, 60 amino acids, 65 amino acids, 70 amino acids, or 75 amino acids. These lengths qualify as short spacers.

In particular implementations, a spacer is 71 amino acids, 75 amino acids, 80 amino acids, 85 amino acids, 90 amino acids, 95 amino acids, 100 amino acids, 110 amino acids, 120 amino acids, 125 amino acids, 128 amino acids, 131 amino acids, 135 amino acids, 140 amino acids, 150 amino acids, 160 amino acids, or 179 amino acids. These lengths qualify as intermediate spacers.

In particular implementations, a spacer is 180 amino acids, 190 amino acids, 200 amino acids, 210 amino acids, 212 amino acids, 214 amino acids, 216 amino acids, 218 amino acids, 220 amino acids, 228 amino acids, 230 amino acids, 240 amino acids, 250 amino acids, 260 amino acids, or 270 amino acids. These lengths qualify as long spacers.

Exemplary spacers include all or a portion of an immunoglobulin hinge region. An immunoglobulin hinge region may be a wild-type immunoglobulin hinge region or an altered wild-type immunoglobulin hinge region. In certain implementations, an immunoglobulin hinge region is a human immunoglobulin hinge region. As used herein, a “wild type immunoglobulin hinge region” refers to a naturally occurring upper and middle hinge amino acid sequences interposed between and connecting the CH1 and CH2 domains (for IgG, IgA, and IgD) or interposed between and connecting the CH1 and CH3 domains (for IgE and IgM) found in the heavy chain of an antibody.

An immunoglobulin hinge region may be an IgG, IgA, IgD, IgE, or IgM hinge region. An IgG hinge region may be an IgG1, IgG2, IgG3, or IgG4 hinge region. Sequences from IgG1, IgG2, IgG3, IgG4 or IgD can be used alone or in combination with all or a portion of a CH2 region; all or a portion of a CH3 region; or all or a portion of a CH2 region and all or a portion of a CH3 region.

In particular implementations, the spacer is a short spacer including an IgG4 hinge region. In particular implementations, the short spacer is an IgG4 hinge S10P. In particular implementations, the spacer is an intermediate spacer including an IgG4 hinge region and an IgG4 CH3 region. In particular implementations, the spacer is a long spacer including an IgG4 hinge region, an IgG4 CH2 region, and an IgG4 CH3 region. In various cases, the spacer in the extracellular component 212 includes at least one sequence having at least 60%, 70%, 80%, 90%, 95%, 98%, or 99% sequence identity to SEQ ID NO: 7, SEQ ID NO: 8, or SEQ ID NO: 9.

Other examples of hinge regions that can be used in the CAR 204 described herein include the hinge region present in the extracellular regions of type 1 membrane proteins, such as CD8α, CD4, CD28 and CD7, which may be wild-type or variants thereof.

In particular implementations, a spacer includes a hinge region that includes a type II C-lectin interdomain (stalk) region or a cluster of differentiation (CD) molecule stalk region. A “stalk region” of a type II C-lectin or CD molecule refers to the portion of the extracellular domain (ECD) of the type II C-lectin or CD molecule that is located between the C-type lectin-like domain (CTLD; e.g., similar to CTLD of natural killer cell receptors) and the hydrophobic portion (transmembrane domain). For example, the ECD of human CD94 (GenBank Accession No. AAC50291.1) corresponds to amino acid residues 34-179, but the CTLD corresponds to amino acid residues 61-176, so the stalk region of the human CD94 molecule includes amino acid residues 34-60, which are located between the hydrophobic portion (transmembrane domain) and CTLD (see Boyington et al., Immunity 10:15, 1999; for descriptions of other stalk regions, see also Beavil et al., Proc. Nat'l. Acad. Sci. USA 89:153, 1992; and Figdor et al., Nat. Rev. Immunol. 2:11, 2002). These type II C-lectin or CD molecules may also have junction amino acids (described below) between the stalk region and the transmembrane region or the CTLD. In another example, the 233 amino acid human NKG2A protein (GenBank Accession No. P26715.1) has a hydrophobic portion (transmembrane domain) ranging from amino acids 71-93 and an ECD ranging from amino acids 94-233. The CTLD includes amino acids 119-231 and the stalk region includes amino acids 99-116, which may be flanked by additional junction amino acids. Other type II C-lectin or CD molecules, as well as their extracellular ligand-binding domains, stalk regions, and CTLDs are known in the art (see, e.g., GenBank Accession Nos. NP 001993.2; AAH07037.1; NP 001773.1; AAL65234.1; CAA04925.1; for the sequences of human CD23, CD69, CD72, NKG2A, and NKG2D and their descriptions, respectively).

According to some cases, the CAR 204 includes one or more linkers. As used herein, a linker can include a chemical moiety that serves to connect two other subcomponents of the molecule. Some linkers serve no purpose other than to link components while many linkers serve an additional purpose. Linkers can, for example, link VL and VH of antibody derived binding domains of scFvs and serve as junction amino acids between subcomponent portions of the CAR 204.

Linkers can be flexible, rigid, or semi-rigid, depending on the desired function of the linker. Linkers can include junction amino acids. For example, in particular implementations, linkers provide flexibility and room for conformational movement between different components of the CAR 204. Commonly used flexible linkers include Gly-Ser linkers. In particular implementations, the linker sequence includes sets of glycine and serine repeats such as from one to ten repeats of (GlyₓSer_{y})ₙ, wherein x and y are independently an integer from 0 to 10 provided that x and y are not both 0 and wherein n is an integer of 1, 2, 3, 4, 5, 6, 7, 8, 9 or 10).

In particular implementations, a linker region is (GGGGS)n (SEQ ID NO: 38) wherein n is an integer including, 1, 2, 3, 4, 5, 6, 7, 8, 9, or more. In particular implementations, the spacer is (EAAAK)n (SEQ ID NO: 39) wherein n is an integer including 1, 2, 3, 4, 5, 6, 7, 8, 9, or more.

In some situations, flexible linkers may be incapable of maintaining a distance or positioning of CAR needed for a particular use. In these instances, rigid or semi-rigid linkers may be useful. Examples of rigid or semi-rigid linkers include proline-rich linkers. In particular implementations, a proline-rich linker is a peptide sequence having more proline residues than would be expected based on chance alone. In particular implementations, a proline-rich linker is one having at least 30%, at least 35%, at least 36%, at least 39%, at least 40%, at least 48%, at least 50%, or at least 51% proline residues. Particular examples of proline-rich linkers include fragments of proline-rich salivary proteins (PRPs).

Linkers can be susceptible to cleavage (cleavable linker), such as, acid-induced cleavage, photo-induced cleavage, peptidase-induced cleavage, esterase-induced cleavage, and disulfide bond cleavage. Alternatively, linkers can be substantially resistant to cleavage (e.g., stable linker or noncleavable linker). In some aspects, the linker is a procharged linker, a hydrophilic linker, or a dicarboxylic acid-based linker.

Junction amino acids can be a linker which can be used to connect sequences when the distance provided by a spacer is not needed and/or wanted. For example, junction amino acids can be short amino acid sequences that can be used to connect co-stimulatory intracellular signaling components. In particular implementations, junction amino acids are 9 amino acids or less (e.g., 2, 3, 4, 5, 6, 7, 8, or 9 amino acids). In particular implementations, a glycine-serine doublet can be used as a suitable junction amino acid linker. In particular implementations, a single amino acid, e.g., an alanine, a glycine, can be used as a suitable junction amino acid.

In particular implementations, the CAR 204 can include one or more tag cassettes and/or transduction markers. Tag cassettes and transduction markers can be used to activate, promote proliferation of, detect, enrich for, isolate, track, deplete and/or eliminate genetically modified cells in vitro, in vivo and/or ex vivo. “Tag cassette” refers to a unique synthetic peptide sequence affixed to, fused to, or that is part of a CAR, to which a cognate binding molecule (e.g., ligand, antibody, or other binding partner) is capable of specifically binding where the binding property can be used to activate, promote proliferation of, detect, enrich for, isolate, track, deplete and/or eliminate the tagged protein and/or cells expressing the tagged protein. Transduction markers can serve the same purposes but are derived from naturally occurring molecules and are often expressed using a skipping element that separates the transduction marker from the rest of the CAR molecule.

Tag cassettes that bind cognate binding molecules include, for example, His tag (HHHHHH; SEQ ID NO: 40), Flag tag (DYKDDDDK; SEQ ID NO: 41), Xpress tag (DLYDDDDK; SEQ ID NO: 42), Avi tag (GLNDIFEAQKIEWHE; SEQ ID NO: 43), Calmodulin tag (KRRWKKNFIAVSAANRFKKISSSGAL; SEQ ID NO: 44), Polyglutamate tag, HA tag (YPYDVPDYA; SEQ ID NO: 45), Myc tag (EQKLISEEDL; SEQ ID NO: 46), Strep tag (which refers the original STREP® tag (WRHPQFGG; SEQ ID NO: 47), STREP® tag II (WSHPQFEK SEQ ID NO: 48 (IBA Institut fur Bioanalytik, Germany); see, e.g., U.S. Pat. No. 7,981,632), Softag 1 (SLAELLNAGLGGS; SEQ ID NO: 49), Softag 3 (TQDPSRVG; SEQ ID NO: 50), and V5 tag (GKPIPNPLLGLDST; SEQ ID NO: 51).

Conjugate binding molecules that specifically bind tag cassette sequences disclosed herein are commercially available. For example, His tag antibodies are commercially available from suppliers including Life Technologies, Pierce Antibodies, and GenScript.Flag tag antibodies are commercially available from suppliers including Pierce Antibodies, GenScript, and Sigma-Aldrich. Xpress tag antibodies are commercially available from suppliers including Pierce Antibodies, Life Technologies and GenScript. Avi tag antibodies are commercially available from suppliers including Pierce Antibodies, IsBio, and Genecopoeia. Calmodulin tag antibodies are commercially available from suppliers including Santa Cruz Biotechnology, Abcam, and Pierce Antibodies. HA tag antibodies are commercially available from suppliers including Pierce Antibodies, Cell Signal and Abcam. Myc tag antibodies are commercially available from suppliers including Santa Cruz Biotechnology, Abcam, and Cell Signal. Strep tag antibodies are commercially available from suppliers including Abcam, Iba, and Qiagen.

Transduction markers may be selected from at least one of a truncated CD19 (tCD19; see Budde et al., Blood 122:1660, 2013); a truncated human EGFR (tEGFR or EGFRt; see Wang et al., Blood 118:1255, 2011); an ECD of human CD34; and/or RQR8 which combines target epitopes from CD34 (see Fehse et al, Mol. Therapy 1 (5 Pt 1); 448-456, 2000) and CD20 antigens (see Philip et al, Blood 124:1277-1278). In particular implementations, cells are genetically modified to express tCD19. In some implementations, the CAR 204 expresses EGFRt.

In particular implementations, the CAR 204 can include a polynucleotide that encodes a self-cleaving polypeptide, wherein the polynucleotide encoding the self-cleaving polypeptide is located between the polynucleotide encoding the CAR 204 and a polynucleotide encoding a transduction marker (e.g., EGFRt). Exemplary self-cleaving polypeptides include 2A peptide from porcine teschovirus-1 (P2A), Thosea asigna virus (T2A), equine rhinitis A virus (E2A), foot-and-mouth disease virus (F2A), or variants thereof. Further exemplary nucleic acid and amino acid sequences of 2A peptides are set forth in, for example, Kim et al. (PLOS One 6:e18556 (2011). In particular implementations, cells are genetically modified to include a self-cleaving polypeptide. In particular implementations, the self-cleaving polypeptide includes T2A. In particular implementations, the self-cleaving polypeptide separates the expressed CAR from a transduction marker.

Control features may be present in multiple copies in the CAR 204 or can be expressed as distinct molecules with the use of a skipping element. For example, the CAR 204 can have one, two, three, four or five tag cassettes and/or one, two, three, four, or five transduction markers could also be expressed. For example, implementations can include a CAR 204 construct having two Myc tag cassettes, or a His tag and an HA tag cassette, or a HA tag and a Softag 1 tag cassette, or a Myc tag and a SBP tag cassette. Exemplary transduction markers and cognate pairs are described in U.S. Ser. No. 13/463,247.

One advantage of including at least one control feature in the CAR 204 is that cells expressing the CAR 204 administered to a subject can be increased or depleted using the cognate binding molecule to a tag cassette. In certain implementations, the present disclosure provides a method for depleting a modified cell expressing the CAR 204 by using an antibody specific for the tag cassette, using a cognate binding molecule specific for the control feature, or by using a second modified cell expressing the CAR 204 and having specificity for the control feature. Elimination of modified cells may be accomplished using depletion agents specific for a control feature. For example, if tCD19 is used, then an anti-tCD19 binding domain (e.g., antibody, scFv) fused to or conjugated to a cell-toxic reagent (such as a toxin, radiometal) may be used, or an anti-tCD19/anti-CD3 bispecific scFv, or an anti-CD19 CAR T-cell may be used.

In particular implementations, a polynucleotide encoding an iCaspase9 construct (iCasp9) may be inserted into a CAR construct as a suicide switch.

In certain implementations, modified cells expressing the CAR 204 may be detected or tracked in vivo by using antibodies that bind with specificity to a control feature (e.g., anti-Tag antibodies), or by other cognate binding molecules that specifically bind the control feature, which binding partners for the control feature are conjugated to a fluorescent dye, radio-tracer, iron-oxide nanoparticle or other imaging agent known in the art for detection by X-ray, CT-scan, MRI-scan, PET-scan, ultrasound, flow-cytometry, near infrared imaging systems, or other imaging modalities (see, e.g., Yu, et al., Theranostics 2:3, 2012).

Thus, modified cells expressing at least one control feature with the CAR 204 can be, e.g., more readily identified, isolated, sorted, induced to proliferate, tracked, and/or eliminated as compared to a modified cell without a tag cassette.

In particular cases, cells are genetically engineered to express the CAR 204. In particular implementations, the engineered cells can be assessed for surface expression of the CAR 204. In particular implementations, at least 50%, at least 55%, at least 60%, at least 65%, at least 70%, at least 75%, at least 80%, at least 85%, at least 90%, at least 95%, or at least 99% of the engineered cells express a detectable level of the CAR.

Surface protein expression can be determined by flow cytometry using methods known in the art. By labeling a population of cells with an element that targets the desired cell surface marker (e.g., an antibody) and is tagged with a fluorescent molecule, flow cytometry can be used to quantify the portion of the population that is positive for the surface marker, as well as the level of surface marker expression.

Genomic incorporation of the CAR 204 within engineered cells can be determined by digital droplet PCR (ddPCR). Digital PCR enables quantification of DNA concentration in a sample. Digital PCR is performed by fractionating a mixture of a PCR reaction (e.g., containing a sample of nucleic acid molecules and copies of a PCR probe) such that some fractions contain no PCR probe copy, while other fractions contain one or more PCR probe copies. A PCR amplification of the fractions is performed and the fractions are analyzed for a PCR reaction. A fraction containing one or more probes and one or more target DNA molecules yields a positive end-point, while a fraction containing no PCR probe yields a negative end-point. The fraction of positive reactions can then be fitted to a Poisson distribution to determine the absolute copy number of target DNA molecules per given volume of the unfractionated sample (i.e., copies per microliter of sample) (see Hindson, B. et al., (2011) Anal Chem. 83:8604-8610). Digital droplet PCR is a variation of digital PCR wherein a sample of nucleic acids is fractionated into droplets using a water-oil emulsion. PCR amplification is performed on the droplets collectively, whereupon a fluidics system is used to separate the droplets and provide analysis of each individual droplet. For one skilled in the art, ddPCR is used to provide an absolute quantification of DNA in a sample, to perform a copy number variation analysis, or to assess efficiency of genomic edits.

Engineered cells can also be assessed for cytokine-independent growth. Engineered cells are expected to only grow in the presence of stimulatory cytokines (e.g., IL-2, IL-7). Growth in the absence of cytokines is an indicator of tumorigenic potential. In particular implementations, engineered cells are grown for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days in either the presence or in the absence of one or more stimulatory cytokines (e.g., IL-2, IL-7). In particular implementations, proliferation is assessed by cell count and viability using conventional methods (e.g., flow cytometry, microscopy, optical density, metabolic activity). In particular implementations, proliferation is assessed starting on day 1, day 2, day 3, day 4, day 5, day 6. In particular implementations, proliferation is assessed every 1 day, every 2 days, every 3 days, every 4 days, every 5 days, every 6 days, every 7 days, or every 8 days. In particular implementations, growth in the absence of cytokines is assessed at the end of a growth period. In some particular implementations, engineered cells with no growth in the absence of cytokines is defined as lacking tumorigenic potential. In particular implementations, no growth is defined as an expansion of the population that is less than 0.1, 0.2, 0.3, 0.4, 0.5, 0.6, 0.7, 0.8, 0.9, 1.0, 1.1, 1.2, 1.3, 1.4, or 1.5 fold between the end of the growth period relative to the beginning of the growth period. In particular implementations, the engineered cells do not proliferate in the absence of cytokine stimulation, growth factor stimulation, or antigen stimulation.

Cell populations can be incubated in a culture-initiating composition to expand cell populations. The incubation can be carried out in a culture vessel, such as a bag, cell culture plate, flask, chamber, chromatography column, cross-linked gel, cross-linked polymer, column, culture dish, hollow fiber, microtiter plate, silica-coated glass plate, tube, tubing set, well, vial, or other container for culture or cultivating cells.

In particular implementations, the cell population can be incubated in the culture-initiating composition before or after genetic engineering the cell populations. In particular implementations, the incubation can be carried out for 1 day to 6 days, 1 day to 5 days, 1 day to 4 days, 1 day to 3 days, 1 day to 2 days, or 1 day before genetically engineering the cell populations. In particular implementations, the incubation can be carried out for 1 day to 6 days, 1 day to 5 days, 1 day to 4 days, 1 day to 3 days, 1 day to 2 days, or 1 day after genetically engineering the cell populations. In particular implementations, the incubation can be carried out at the same time as genetically engineering the cell populations.

Culture conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177, Klebanoff et al. (2012) J Immunother. 35(9): 651-660, Terakura et al. (2012) Blood. 1:72-82, and/or Wang et al. (2012) J Immunother. 35(9):689-701.

Exemplary culture media for culturing T-cells, for instance, include (i) RPMI supplemented with non-essential amino acids, sodium pyruvate, and penicillin/streptomycin; (ii) RPMI with HEPES, 5-15% human serum, 1-3% L-Glutamine, 0.5-1.5% penicillin/streptomycin, and 0.25×10-4−0.75×10-4 M β-MercaptoEthanol; (iii) RPMI-1640 supplemented with 10% fetal bovine serum (FBS), 2 mM L-glutamine, 10 mM HEPES, 100 U/ml penicillin and 100 m/mL streptomycin; (iv) DMEM medium supplemented with 10% FBS, 2 mM L-glutamine, 10 mM HEPES, 100 U/ml penicillin and 100 m/mL streptomycin; and (v) X-Vivo 15 medium (Lonza, Walkersville, MD) supplemented with 5% human AB serum (Gemcell, West Sacramento, CA), 1% HEPES (Gibco, Grand Island, NY), 1% Pen-Strep (Gibco), 1% GlutaMax (Gibco), and 2% N-acetyl cysteine (Sigma-Aldrich, St. Louis, MO). T-cell culture media are also commercially available from Hyclone (Logan, UT). Additional T-cell activating components that can be added to such culture media are described in more detail below.

In some implementations, the T-cells are expanded by adding to the culture-initiating composition feeder cells, such as non-dividing peripheral blood mononuclear cells (PBMC), (e.g., such that the resulting population of cells contains at least 5, 10, 20, or 40 or more PBMC feeder cells for each T lymphocyte in the initial population to be expanded); and incubating the culture (e.g., for a time sufficient to expand the numbers of T-cells). In some aspects, the non-dividing feeder cells can include gamma-irradiated PBMC feeder cells. In some implementations, the PBMC are irradiated with gamma rays in the range of 3000 to 3600 rads to prevenT-cell division. In some aspects, the feeder cells are added to culture medium prior to the addition of the populations of T-cells.

Optionally, the incubation may further include adding non-dividing EBV-transformed lymphoblastoid cells (LCL) as feeder cells. LCL can be irradiated with gamma rays in the range of 6000 to 10,000 rads. The LCL feeder cells in some aspects is provided in any suitable amount, such as a ratio of LCL feeder cells to initial T lymphocytes of at least 10:1.

In some implementations, the stimulating conditions include temperature suitable for the growth of human T lymphocytes, for example, at least 25 °C, at least 30 °C, or 37 °C.

The activating culture conditions for T-cells include conditions whereby T-cells of the culture-initiating composition proliferate or expand. T-cell activating conditions can include one or more cytokines, for example, interleukin (IL)-2, IL-7, IL-15 and/or IL-21. IL-2 can be included at a range of 10-100 ng/ml (e.g., 40, 50, or 60 ng/ml). IL-7, IL-15, and/or IL-21 can be individually included at a range of 0.1-50 ng/ml (e.g., 5, 10, or 15 ng/ml). Particular implementations utilize IL-2 at 50 ng/ml. Particular implementations utilize, IL-7, IL-15 and IL-21 individually included at 10 ng/ml.

In particular implementations, T-cell activating culture condition conditions can include T-cell stimulating epitopes. T-cell stimulating epitopes include CD3, CD27, CD2, CD4, CD5, CD7, CD8, CD28, CD30, CD40, CD56, CD83, CD90, CD95, 4-1BB (CD 137), B7-H3, CTLA-4, Frizzled-1 (FZD1), FZD2, FZD3, FZD4, FZD5, FZD6, FZD7, FZD8, FZD9, FZD10, HVEM, ICOS, IL-1R, LAT, LFA-1, LIGHT, MHCI, MHCII, NKG2D, OX40, ROR2 and RTK.

CD3 is a primary signal transduction element of T-cell receptors. As indicated previously, CD3 is expressed on all mature T-cells. In particular implementations, the CD3 stimulating molecule (i.e., CD3 binding domain) can be derived from the OKT3 antibody (see U.S. Pat. Nos. 5,929,212; 4,361,549; ATCC® CRL-8001™; and Arakawa et al., J. Biochem. 120, 657-662 (1996)), the 20G6-F3 antibody, the 4B4-D7 antibody, the 4E7-C9, or the 18F5-H10 antibody.

In particular implementations, CD3 stimulating molecules can be included within culture media at a concentration of at least 0.25 or 0.5 ng/ml or at a concentration of 2.5-10 μg/ml. Particular implementations utilize a CD3 stimulating molecule (e.g., OKT3) at 5 μg/ml.

In particular implementations, activating molecules associated with avi-tags can be biotinylated and bound to streptavidin beads. This approach can be used to create, for example, a removable T-cell epitope stimulating activation system.

An exemplary binding domain for CD28 can include or be derived from TGN1412, CD80, CD86 or the 9D7 antibody. Additional antibodies that bind CD28 include 9.3, KOLT-2, 15E8, 248.23.2, EX5.3D10, and CD28.3 (deposited as a synthetic single chain Fv construct under GenBank Accession No. AF451974.1; see also Vanhove et al., BLOOD, 15 Jul. 2003, Vol. 102, No. 2, pages 564-570). Further, 1YJD provides a crystal structure of human CD28 in complex with the Fab fragment of a mitogenic antibody (5.11A1). In particular implementations, antibodies that do not compete with 9D7 are selected.

4-1BB binding domains can be derived from LOB12, IgG2a, LOB12.3, or IgG1 as described in Taraban et al. Eur J Immunol. 2002 December; 32(12):3617-27. In particular implementations a 4-1BB binding domain is derived from a monoclonal antibody described in U.S. Pat. No. 9,382,328. Additional 4-1BB binding domains are described in U.S. Pat. Nos. 6,569,997, 6,303,121, and Mittler et al. Immunol Res. 2004; 29(1-3):197-208.

OX40 (CD134) and/or ICOS activation may also be used. OX40 binding domains are described in US20100196359, US20150307617, WO 2015/153513, WO2013/038191 and Melero et al. Clin Cancer Res. 2013 Mar. 1; 19(5):1044-53. Exemplary binding domains that can bind and activate ICOS are described in e.g., US20080279851 and Deng et al. Hybrid Hybridomics. 2004 June; 23(3):176-82.

When in soluble form, T-cell activating agents can be coupled with another molecule, such as polyethylene glycol (PEG) molecule. Any suitable PEG molecule can be used. Typically, PEG molecules up to a molecular weight of 1000 Da are soluble in water or culture media. In some cases, such PEG based reagent can be prepared using commercially available activated PEG molecules (for example, PEG-NHS derivatives available from NOF North America Corporation, Irvine, Calif., USA, or activated PEG derivatives available from Creative PEGWorks, Chapel Hills, N.C., USA).

In particular implementations, cell stimulating agents are immobilized on a solid phase within the culture media. In particular implementations, the solid phase is a surface of the culture vessel (e.g., bag, cell culture plate, chamber, chromatography column, cross-linked gel, cross-linked polymer, column, culture dish, hollow fiber, microtiter plate, silica-coated glass plate, tube, tubing set, well, vial, other structure or container for culture or cultivation of cells).

In particular implementations, a solid phase can be added to a culture media. Such solid phases can include, for example, beads, hollow fibers, resins, membranes, and polymers.

Exemplary beads include magnetic beads, polymeric beads, and resin beads (e.g., Strep-Tactin® Sepharose, Strep-Tactin® Superflow, and Strep-Tactin® MacroPrep IBA GmbH, Gottingen). Anti-CD3/anti-CD28 beads are commercially available reagents for T-cell expansion (Invitrogen). These beads are uniform, 4.5 μm superparamagnetic, sterile, non-pyrogenic polystyrene beads coated with a mixture of affinity purified monoclonal antibodies against the CD3 and CD28 cell surface molecules on human T-cells. Hollow fibers are available from TerumoBCT Inc. (Lakewood, Colo., USA). Resins include metal affinity chromatography (IMAC) resins (e.g., TALON® resins (Westburg, Leusden)). Membranes include paper as well as the membrane substrate of a chromatography matrix (e.g., a nitrocellulose membrane or a polyvinylidene difluoride (PVDF) membrane).

Exemplary polymers include polysaccharides, such as polysaccharide matrices. Such matrices include agarose gels (e.g., Superflow™ agarose or a Sepharose® material such as Superflow™ Sepharose® that are commercially available in different bead and pore sizes) or a gel of crosslinked dextran(s). A further illustrative example is a particulate cross-linked agarose matrix, to which dextran is covalently bonded, that is commercially available (in various bead sizes and with various pore sizes) as Sephadex® or Superdex®, both available from GE Healthcare.

Synthetic polymers that may be used include polyacrylamide, polymethacrylate, a co-polymer of polysaccharide and agarose (e.g. a polyacrylamide/agarose composite) or a polysaccharide and N,N′-methylenebisacrylamide. An example of a copolymer of a dextran and N,N′-methylenebisacrylamide is the Sephacryl® (Pharmacia Fine Chemicals, Inc., Piscataway, NJ) series of materials.

Particular implementations may utilize silica particles coupled to a synthetic or to a natural polymer, such as polysaccharide grafted silica, polyvinylpyrrolidone grafted silica, polyethylene oxide grafted silica, poly(2-hydroxyethylaspartamide) silica and poly(N-isopropylacrylamide) grafted silica.

Cell activating agents can be immobilized to solid phases through covalent bonds or can be reversibly immobilized through non-covalent attachments.

In particular implementations, a T-cell activating culture media includes a FACS-sorted T-cell population cultured within RPMI with HEPES, 5-15% human serum, 1-3% L-Glutamine, 0.5-1.5% Pen/strep, 0.25×10⁻⁴−0.75×10⁻⁴ M β-MercaptoEthanol, with IL-7, IL-15 and IL-21 individually included at 5-15 (e.g., 10) ng/ml. The culture is carried out on a flat-bottom well plate with 0.1-0.5×10⁶ plated cells/well. On Day 3 post activation cells are transferred to a tissue culture (TC)-treated plate.

In particular implementations, a T-cell activating culture media includes a FACS-sorted CD8+ T population cultured within RPMI with HEPES, 10% human serum, 2% L-Glutamine, 1% Pen/strep, 0.5×10⁻⁴ M β-MercaptoEthanol, with IL-7, IL-15 and IL-21 individually included at 5-15 (e.g., 10) ng/ml. The culture is carried out on a flat-bottom non-tissue culture-treated 96/48-well plate with 0.1-0.5×10⁶ plated cells/well. On Day 3 post activation cells are transferred to TC-treated plate.

Culture conditions for HSC/HSP can include expansion with a Notch agonist (see, e.g., U.S. Pat. Nos. 7,399,633; 5,780,300; 5,648,464; 5,849,869; and 5,856,441 and growth factors present in the culture condition as follows: 25-300 ng/ml SCF, 25-300 ng/ml Flt-3L, 25-100 ng/ml TPO, 25-100 ng/ml IL-6 and 10 ng/ml IL-3. In more specific implementations, 50, 100, or 200 ng/ml SCF; 50, 100, or 200 ng/ml of Flt-3L; 50 or 100 ng/ml TPO; 50 or 100 ng/ml IL-6; and 10 ng/ml IL-3 can be used.

In particular implementations, genetically modified cells can be harvested from a culture medium and washed and concentrated into a carrier in a therapeutically-effective amount. As described herein, exemplary carriers include saline, buffered saline, physiological saline, water, Hanks' solution, Ringer's solution, Normosol-R (Abbott Labs), PLASMA-LYTE A® (Baxter Laboratories, Inc., Morton Grove, IL), and combinations thereof.

Cells and or other components described herein (e.g., the synthetic intermediate 202) may be administered in a formulation that includes one or more carriers, stabilizers, anesthetics, preservatives, or any combinations thereof.

In particular implementations, carriers can be supplemented with human serum albumin (HSA) or other human serum components or fetal bovine serum. In particular implementations, a carrier for infusion includes buffered saline with 5% HSA or dextrose. Additional isotonic agents include polyhydric sugar alcohols including trihydric or higher sugar alcohols, such as glycerin, erythritol, arabitol, xylitol, sorbitol, or mannitol.

Carriers can include buffering agents, such as citrate buffers, succinate buffers, tartrate buffers, fumarate buffers, gluconate buffers, oxalate buffers, lactate buffers, acetate buffers, phosphate buffers, histidine buffers, and/or trimethylamine salts.

Stabilizers refer to a broad category of excipients which can range in function from a bulking agent to an additive which helps to prevent cell adherence to container walls. Typical stabilizers can include polyhydric sugar alcohols; amino acids, such as arginine, lysine, glycine, glutamine, asparagine, histidine, alanine, ornithine, L-leucine, 2-phenylalanine, glutamic acid, and threonine; organic sugars or sugar alcohols, such as lactose, trehalose, stachyose, mannitol, sorbitol, xylitol, ribitol, myoinisitol, galactitol, glycerol, and cyclitols, such as inositol; PEG; amino acid polymers; sulfur-containing reducing agents, such as urea, glutathione, thioctic acid, sodium thioglycolate, thioglycerol, alpha-monothioglycerol, and sodium thiosulfate; low molecular weight polypeptides (i.e., <10 residues); proteins such as HSA, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran.

Where necessary or beneficial, formulations can include a local anesthetic such as lidocaine to ease pain at a site of injection.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol, and 3-pentanol.

Therapeutically effective amounts of cells within formulations can be greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

In formulations disclosed herein, cells are generally in a volume of a liter or less, 500 ml or less, 250 ml or less or 100 ml or less. Hence the density of administered cells is typically greater than 10⁴ cells/ml, 10⁷ cells/ml or 10⁸ cells/ml.

In particular implementations, formulations can include one or more genetically modified cell types (e.g., modified T-cells, NK cells, or stem cells). Formulations can include different types of genetically-modified cells (e.g., T-cells, NK cells, and/or stem cells in combination).

Different types of genetically-modified cells or cell subsets (e.g., modified T-cells, NK cells, and/or stem cells) can be provided in different ratios e.g., a 1:1:1 ratio, 2:1:1 ratio, 1:2:1 ratio, 1:1:2 ratio, 5:1:1 ratio, 1:5:1 ratio, 1:1:5 ratio, 10:1:1 ratio, 1:10:1 ratio, 1:1:10 ratio, 2:2:1 ratio, 1:2:2 ratio, 2:1:2 ratio, 5:5:1 ratio, 1:5:5 ratio, 5:1:5 ratio, 10:10:1 ratio, 1:10:10 ratio, 10:1:10 ratio, etc. These ratios can also apply to numbers of cells expressing the same or different components of the CAR 204. If only two of the cell types are combined or only 2 combinations of expressed CAR components are included within a formulation, the ratio can include any 2-number combination that can be created from the 3 number combinations provided above. In implementations, the combined cell populations are tested for efficacy and/or cell proliferation in vitro, in vivo and/or ex vivo, and the ratio of cells that provides for efficacy and/or proliferation of cells is selected. Particular implementations include a 1:1 ratio of CD4 T-cells and CD8 T-cells.

The cell-based formulations disclosed herein can be prepared for administration by, e.g., injection, infusion, perfusion, or lavage. The formulations can further be formulated for bone marrow, intravenous, intradermal, intraarterial, intranodal, intralymphatic, intraperitoneal, intralesional, intraprostatic, intravaginal, intrarectal, intrathecal, intramuscular, intravesicular, and/or subcutaneous injection.

In various implementations, the antigen 206 is expressed on a surface of a cell that is targeted by the universal CAR T-cell therapy. For example, the antigen 206 may include DLL3, CLL1, GPRC5D, EpCAM, CD3, CD5, CD7, fibroblast activation protein, GAD65, citrullinated vimentin, myelin oligodendrocyte glycoprotein, carcinoembryonic antigen, prostate specific antigen, Prostate Stem Cell antigen, PSMA, Her2/neu, estrogen receptor, progesterone receptor, ephrinB2, CD19, CD20, CD22, CD23, CD123, CS-1, CE7, ROR1, mesothelin, c-Met, GD-2, EGFR, EGFRvIII, EphA2, IL13Ra2, L1CAM, oaGD2, GD2, B7H3, CD33, VAR2CSA, MUC16, PD-L1, ERBB2, folate receptor, biotin receptor, CD56; glypican-2, disialoganglioside, EpCam, L1-CAM, Lewis Y, WT-1, Tyrosinase related protein 1, GD2, B-cell maturation antigen, CD24, SV40 T, carboxy-anhydrase-IX, or CD133.

The synthetic intermediate 202 includes a tag 216 that specifically binds to the intermediate binding domain 214 of the CAR 204. For example, the tag 216 includes a sequence having at least 60%, 70%, 80%, 90%, 95%, 98%, or 99% sequence identity to SEQ ID NO: 5, SEQ ID NO: 6, SEQ ID NO: 14, SEQ ID NO: 15, SEQ ID NO: 20, SEQ ID NO: 23, SEQ ID NO: 31, SEQ ID NO: 32, SEQ ID NO: 33, SEQ ID NO: 34, SEQ ID NO: 35, SEQ ID NO: 36, or SEQ ID NO: 37. The tag 216, for instance, is based on the intermediate binding domain 214.

The synthetic intermediate 202 also includes an antigen-binding domain 218 that specifically binds to at least a portion of the antigen 206. For example, the antigen-binding domain 218 includes SEQ ID NO: 16, in cases wherein the antigen 206 includes αvβ6. In some cases, the antigen-binding domain includes folate in cases where the antigen 206 includes a folate receptor.

In various implementations of the present disclosure, the synthetic intermediate 202 is smaller than a protein. According to some cases, the synthetic intermediate 202 has a mass in a range of 0.1 kDa to 300 kDa, 0.2 kDa to 200 kDa, 0.5 kDa to 200 kDa, 0.5 kDa to 100 kDa, or 0.5 kDa to 30 kDa. According to some examples, the synthetic intermediate 202 includes a peptide, an aptamer, or a peptide-aptamer chimera. For instance, the synthetic intermediate 202 includes at least one sequence of nucleotides, at least one sequence of amino acids, or a combination thereof.

Methods disclosed herein include treating subjects (humans, non-human primates, veterinary animals (dogs, cats, reptiles, birds, etc.) livestock (horses, cattle, goats, pigs, chickens, etc.) and research animals (monkeys, rats, mice, fish, etc.) with formulations disclosed herein. Treating subjects includes delivering therapeutically effective amounts. Therapeutically effective amounts include those that provide effective amounts, prophylactic treatments and/or therapeutic treatments.

An “effective amount” is the amount of a formulation necessary to result in a desired physiological change in the subject. For example, an effective amount can provide an immunogenic anti-cancer effect. Effective amounts are often administered for research purposes. Effective amounts disclosed herein can cause a statistically significant effect in an animal model or in vitro assay relevant to the assessment of a cancer development or progression. An immunogenic formulation can be provided in an effective amount, wherein the effective amount stimulates an immune response.

A “prophylactic treatment” includes a treatment administered to a subject who does not display signs or symptoms of a cancer or displays only early signs or symptoms of a cancer such that treatment is administered for the purpose of diminishing or decreasing the risk of developing the cancer further. Thus, a prophylactic treatment functions as a preventative treatment against a targeted antigen-expressing cancer.

A “therapeutic treatment” includes a treatment administered to a subject who displays symptoms or signs of a cancer and is administered to the subject for the purpose of diminishing or eliminating those signs or symptoms of the cancer. The therapeutic treatment can reduce, control, or eliminate the presence or activity of the cancer and/or reduce control or eliminate side effects of the cancer.

Function as an effective amount, prophylactic treatment or therapeutic treatment are not mutually exclusive, and in particular implementations, administered dosages may accomplish more than one treatment type.

In particular implementations, therapeutically effective amounts provide anti-cancer effects. Anti-cancer effects include a decrease in the number of cancer cells, an increase in life expectancy, induced chemo- or radiosensitivity in cancer cells, inhibited cancer cell proliferation, prolonged subject life, reduced cancer-associated pain, and/or reduced relapse or re-occurrence of cancer following treatment.

For administration, therapeutically effective amounts (also referred to herein as doses or dosages) can be initially estimated based on results from in vitro assays and/or animal model studies. Such information can be used to more accurately determine useful doses in subjects of interest. The actual dose amount administered to a particular subject can be determined by a physician, veterinarian or researcher taking into account parameters such as physical and physiological factors including target, body weight, severity of condition, type of cancer, stage of cancer, previous or concurrent therapeutic interventions, idiopathy of the subject and route of administration.

Therapeutically effective amounts of cell-based formulations (e.g., of cells expressing the CAR 204) can include 10⁴ to 10⁹ cells/kg body weight, or 103 to 10¹¹ cells/kg body weight. Therapeutically effective amounts to administer can include greater than 10² cells, greater than 10³ cells, greater than 10⁴ cells, greater than 10⁵ cells, greater than 10⁶ cells, greater than 10⁷ cells, greater than 10⁸ cells, greater than 10⁹ cells, greater than 10¹⁰ cells, or greater than 10¹¹.

Therapeutically effective amounts of intermediates (e.g., the synthetic intermediate 202) within modifying formulations can range from 0.1 to 5 μg/kg or from 0.5 to 1 μg/kg. In other examples, a dose can include 1 μg/kg, 30 μg/kg, 90 μg/kg, 150 μg/kg, 500 μg/kg, 750 μg/kg, 0.1 to 5 mg/kg or from 0.5 to 1 mg/kg. In other examples, a dose can include 1 mg/kg, 10 mg/kg, 30 mg/kg, 50 mg/kg, 70 mg/kg, 100 mg/kg, 300 mg/kg, 500 mg/kg, 700 mg/kg, 1000 mg/kg or more.

Therapeutically effective amounts can be achieved by administering single or multiple doses during the course of a treatment regimen (e.g., daily, every other day, every 3 days, every 4 days, every 5 days, every 6 days, weekly, every 2 weeks, every 3 weeks, monthly, every 2 months, every 3 months, every 4 months, every 5 months, every 6 months, every 7 months, every 8 months, every 9 months, every 10 months, every 11 months or yearly). In particular implementations, the treatment protocol may be dictated by a clinical trial protocol or an FDA-approved treatment protocol.

Therapeutically effective amounts can be administered by, e.g., injection, infusion, perfusion, or lavage. Routes of administration can include bolus intravenous, intradermal, intraarterial, intraparenteral, intranodal, intralymphatic, intraperitoneal, intralesional, intraprostatic, intravaginal, intrarectal, topical, intrathecal, intramuscular, intravesicular, and/or subcutaneous administration.

In some implementations, the CAR 204, the synthetic intermediate 202, and other elements described herein can be included in one or more kits. Kits can include various components to practice methods disclosed herein. For example, depending on the aspect of the methods practiced, kits could include one or more of nucleic acids encoding the CAR 204, the synthetic intermediate 202, a nucleic acid encoding an scFv; a nucleic acid encoding a VL; a nucleic acid encoding a VH; a nucleic acid encoding a transmembrane domain; a nucleic acid encoding tCD19; cells (e.g., immune cells, T-cells, CD4 T-cells, CD8 T-cells, B cells, natural killer (NK) cells, NK-T-cells, monocytes/macrophages, lymphocytes, hematopoietic stem cells (HSCs), hematopoietic progenitor cells (HPC), and/or a mixture of HSC and HPC (i.e., HSPC), untransduced T-cells, CAR T-cells; cell lines (e.g., REH-1 cell lines); tissue samples (e.g., peripheral blood mononuclear cells (PBMCs), specimens, or other organ, and/or cells derived therefrom); genetic expression components (e.g., genes for expression provided by vectors (e.g., lentiviral vector, retroviral vector), CRISPR components, ZFNs, TALENS, MegaTALs, targeted viral vectors and/or nanoparticles); cell formulations or activation components (e.g., saline, buffered saline, phosphate buffered saline (PBS); biocompatible buffers such as, Ca++/Mg++ free PBS; physiological saline, water, Hanks' solution, Ringer's solution, T-cell stimulating epitopes (e.g., anti-CD3/anti-CD28 conjugated beads; OKT3, TGN1412), culture-initiating compositions, RPMI, non-essential amino acids, sodium pyruvate, penicillin/streptomycin, non-dividing EBV-transformed lymphoblastoid cells (LCL), IL-21, human serum albumin (HSA) or other human serum components or fetal bovine serum, dextrose, Stabilizers, preservatives); components for screening form KMT2A fusion (e.g., fusion probe, KMT2A probe, fluorescent-labeled nucleotide analog, microscope, FISH analytics software); combination therapy components (e.g., local anesthetics, chemotherapeutic agents, immunosuppressive agents, anti-inflammatory agents); an antibody tagged with a fluorescent molecule; PCR amplification sequences; cytokines (e.g., IL-2, IL-7, IL-15, IL-21); culture vessels; GAPDH; IFN-γ enzyme-linked immunosorbent assay (ELISA); culture plates; etc.

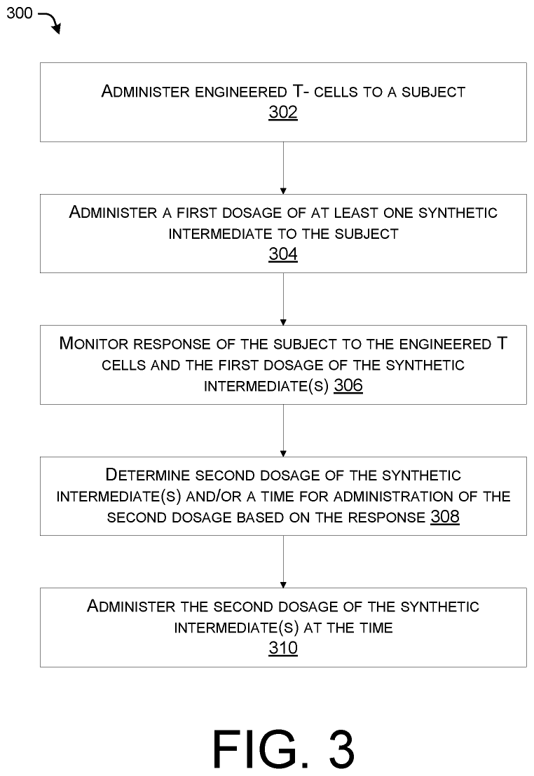

FIG. 3 illustrates an example process 300 for administering a universal CAR T-cell therapy to a subject using at least one synthetic intermediate. The process 300 is performed by an entity, which may include one or more of an intravenous pump, a subcutaneous device, a computing device, a fluidic device, or a care provider. According to some implementations, any of the steps of the process 300 may be omitted.

At 302, the entity administers engineered T-cells to a subject. In various implementations, the T-cells are engineered to express a CAR. The CAR, for example, includes an extracellular component and an intracellular component linked by a transmembrane domain. The extracellular component, in various cases, includes an intermediate-binding domain. In various cases, the intermediate-binding domain includes a sequence having at least 60%, 70%, 80%, 90%, 95%, 98%, or 99% sequence identity to a sequence as set forth in SEQ ID NO: 5, SEQ ID NO: 6, or SEQ ID NO: 20. In various implementations, the engineered T-cells are derived from T-cells of the subject. In some cases, the engineered T-cells are derived from T-cells of at least one donor who is not the subject. The engineered T-cells may be administered to the subject intravenously.

At 304, the entity administers a first dosage of the synthetic intermediate(s) to the subject. In various cases, the entity administers a formulation including the synthetic intermediate(s). The first dosage, for example, may represent a mass or number of copies of the synthetic intermediate(s) in the formulation, a volume of the formulation, or the like. In some cases, the first dosage corresponds to a rate (over time) at which the synthetic intermediate(s) are administered to the subject. The formulation, for instance, is administered subcutaneously and/or intravenously.

In various implementations, the synthetic intermediate(s) include a tag. The tag and the intermediate-binding domain, for example, specifically bind to one another. According to some cases, the tag includes a sequence having at least 60%, 70%, 80%, 90%, 95%, 98%, or 99% sequence identity to SEQ ID NO: 5, SEQ ID NO: 6, SEQ ID NO: 15, SEQ ID NO: 20, SEQ ID NO: 31, SEQ ID NO: 32, SEQ ID NO: 33, SEQ ID NO: 34, SEQ ID NO: 35, SEQ ID NO: 36, or SEQ ID NO: 37.

According to some instances, each synthetic intermediate(s) includes an antigen binding domain. For instance, the antigen binding domain specifically binds at least a portion of an antigen expressed by a target cell in the body of the subject. The antigen may be a cancer antigen and/or a viral antigen. Examples of the antigen include DLL3, CLL1, GPRC5D, EpCAM, CD3, CD5, CD7, fibroblast activation protein, GAD65, citrullinated vimentin, myelin oligodendrocyte glycoprotein, carcinoembryonic antigen, prostate specific antigen, Prostate Stem Cell antigen, PSMA, Her2/neu, estrogen receptor, progesterone receptor, ephrinB2, CD19, CD20, CD22, CD23, CD123, CS-1, CE7, ROR1, mesothelin, c-Met, GD-2, EGFR, EGFRvIII, EphA2, IL13Ra2, L1CAM, oaGD2, GD2, B7H3, CD33, VAR2CSA, MUC16, PD-L1, ERBB2, folate receptor, biotin receptor, CD56; glypican-2, disialoganglioside, EpCam, L1-CAM, Lewis Y, WT-1, Tyrosinase related protein 1, GD2, B-cell maturation antigen, CD24, SV40 T, carboxy-anhydrase-IX, CD133, or any other antigen of a targeted cell.

According to various examples, the synthetic intermediate(s) include a peptide (e.g., branched or unbranched), a DNA aptamer, or a DNA aptamer-peptide chimera. The synthetic intermediate(s) may be smaller than proteins, such as antibodies. For examples, the synthetic intermediate(s) may have a mass in a range of 0.5 kDa to 30 kDa. Accordingly, the synthetic intermediate(s) may be administered subcutaneously. Further, the synthetic intermediate(s) may be transported to an interior cellular layer of a solid tumor of the subject after administration.

At 306, the entity monitors a response of the subject to the engineered T-cells and the first dosage of the synthetic intermediate(s). In various cases, the entity determines whether the subject exhibits an immune response, such as CRS and/or neurotoxicity. In some cases, the entity determines whether target cells (e.g., cancer cells) in the body of the subject have been destroyed.

At 308, the entity determines a second dosage of the synthetic intermediate(s) and/or a time for administration of the second dosage based on the response. In some cases, the second dosage is less than the first dosage and/or the time is a relatively late time if the entity determines that the subject exhibited a negative response (e.g., an immune response). In some examples, the second dosage is greater than the first dosage and/or the time is a relatively early time if the entity determines that the target cells in the body of the subject have not been sufficiently destroyed by the engineered T-cells. At 310, the entity administers the second dosage of the synthetic intermediate(s) at the time.

### First Experimental Example

This Experimental Example provides a universal CAR receptor and bifunctional synthetic targeting intermediates that employ enhanced SpyCatcher003-SpyTag003 reaction kinetics to direct T-cell responses against cancer cells. By linking SpyCatcher003 to standard CAR spacer, transmembrane, and signaling domains, SpyCatcher003 CARs (nicknamed DB5 CARs) were developed. DB5 CARs are shown herein to react efficiently with synthetic SpyTag003 peptides at low-nanomolar concentrations on the order of minutes. For cancer targeting, SpyTag003 was attached to a modified αvβ6-specific peptide via branched peptide design, and the resulting bifunctional peptide intermediate displayed robust bispecific properties, labeling both DB5 CAR-expressing immortalized T-cells and αvβ6⁺ cancer cells with high specificity. In vitro functional assays with the bifunctional peptide intermediate and primary CD4⁺ DB5 CAR T-cells of different spacer lengths demonstrate spacer-dependent αvβ6-directed cytokine release, even against target cells with low αvβ6 expression. Similarly, primary CD8⁺ DB5 CAR T-cells pre-armed with bifunctional peptide show directed killing of cancer cells that express αvβ6. Lastly, an azide-modified SpyTag003 was attached to an octyne-modified cancer-targeting DNA aptamer via copper-free click chemistry. The resulting heterobifunctional aptamer-peptide chimera is shown herein as capable of labeling both DB5 CAR-expressing T-cells and cancer cells with high specificity. These results demonstrate the ability for diverse synthetic materials to be covalently attached to DB5 CAR T-cells and reconstitute a targeted immunotherapy for cancer treatment, paving the way for universal cyborg CAR T-cell therapies.

### Results

Accelerated CAR Reaction Kinetics with SpyCatcher003 and SpyTag003

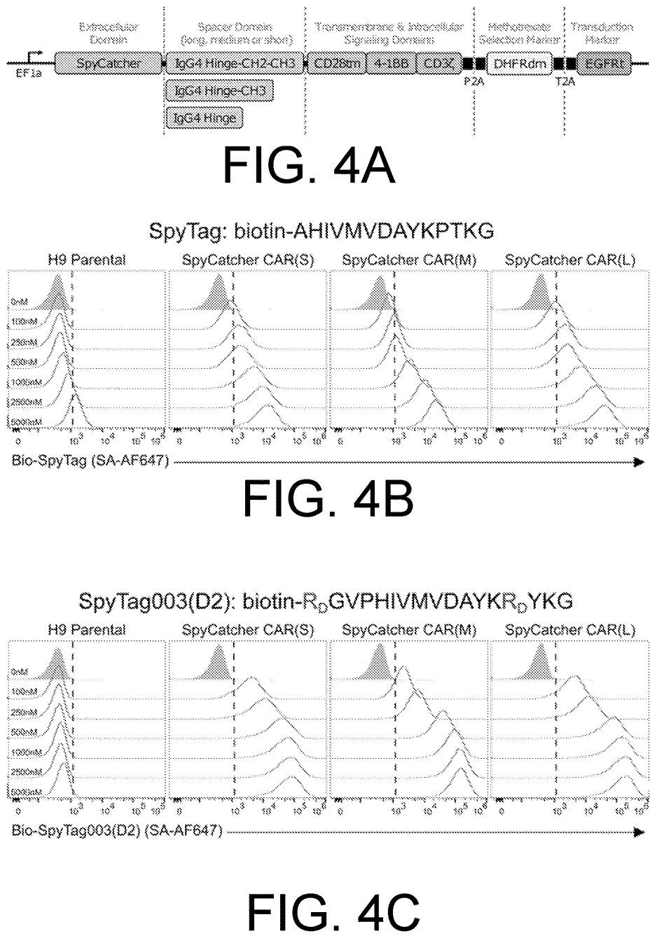

First, a universal CAR system was created based on the original SpyCatcher-SpyTag pair. As the non-signaling extracellular spacer domain is critical for CAR function and the optimal length varies depending on the scFv and targeted epitope (Hudecek, et al., Clinical Cancer Research 2013, 19 (12), 3153-3164), SpyCatcher (113 amino acids, SEQ ID NO: 37) was cloned into three lentiviral CAR constructs with different spacers lengths including either a short IgG4 hinge domain, a medium IgG4 hinge-CH3 domain, or a long IgG4 hinge-CH2-CH3 domain (FIG. 4A). Each spacer was linked to a CD28 transmembrane domain and 41BB-CD3ζ intracellular signaling domains to produce second-generation SpyCatcher-41BB-CD3ζ CARs. Constructs also encoded a double mutant of dihydrofolate reductase (DHFRdm) and truncated EGFR (EGFRt) downstream of the CAR separated by 2A ribosomal skip sequences, allowing for methotrexate drug selection and detection of transduced cells, respectively. To evaluate the covalent reaction kinetics of the SpyCatcher CARs with SpyTag peptide (SEQ ID NO: 14), immortalized T-lymphoma H9 cells were transduced to express the three different SpyCatcher CARs and synthesized biotinylated SpyTag for loading onto the cells. The SpyTag peptide was modified by adding a C-terminal glycine to the SpyTag peptide to mimic a prospective linker that could be added when synthesizing bifunctional peptides.

After incubating biotinylated SpyTag with the cells at various concentrates for 1 h at room temperature and subsequently staining with streptavidin Alexa Fluor 647, low loading of SpyTag peptides onto the SpyCatcher CARs was observed at nanomolar concentrations (FIG. 4B). Ultimately, 5 μM SpyTag peptide was required for moderate covalent loading onto SpyCatcher CARs, which was far from ideal compared to the nanomolar and sub-nanomolar affinities of conventional CARs used in the clinic (Ghorashian, et al., Nature Medicine 2019, 25 (9), 1408-1414). Improved covalent reaction kinetics compared to the original SpyCatcher-SpyTag pair (1.4×10³ M⁻¹ s⁻¹) therefore could be presumed to sensitively and effectively direct T-cell function.

The kinetics of the SpyCatcher and SpyTag interaction has been optimized in recent years, with phage display selection and further rational modification of the pair yielding SpyCatcher003 (SEQ ID NO: 5) and SpyTag003 (SEQ ID NO: 20) that have 400-fold faster reaction kinetics (5.5×10⁵ M⁻¹ s⁻¹) (Keeble, et al., Angewandte Chemie International Edition 2017, 56 (52), 16521-16525; Keeble, PNAS 2019, 116 (52), 26523-26533). As this reaction rate approaches diffusion-limited kinetics, it was hypothesized that a universal CAR system using SpyCatcher003 and SpyTag003 would be significantly improved compared to a system using old SpyCatcher-SpyTag chemistry. Accordingly, loading of the new SpyTag003 peptide onto the previous H9 cells expressing the original SpyCatcher CAR was evaluated, since the new 003 variants are back-compatible with previous SpyCatcher/SpyTag generations but with enhanced kinetics.⁴² In an effort to increase peptide serum stability, a biotinylated SpyTag003 was synthesized with two D-arginine substitutions, which is referred to as SpyTag003(D2) (SEQ ID NO: 15). The arginine residues were originally introduced into SpyTag003 for adding positive charge, so it was rationalized that substituting in the D-configuration of these residues would not affect reaction kinetics.

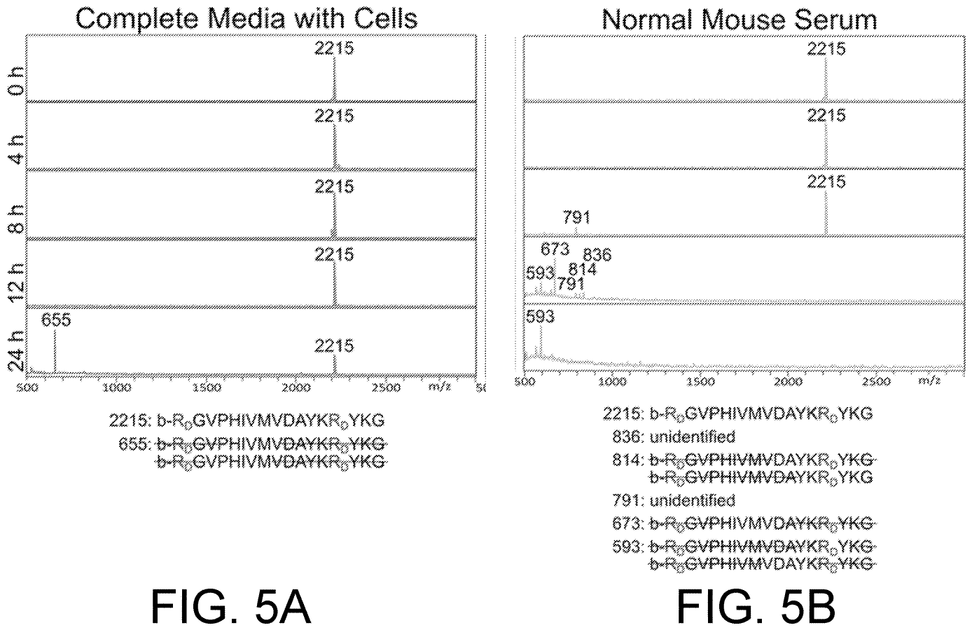

SpyTag003(D2) displayed remarkedly enhanced loading onto H9 SpyCatcher CAR cells, with comparable loading to the original SpyTag peptide occurring at 10-fold lower concentrations (FIG. 4C). SpyTag003(D2) loading onto SpyCatcher CARs saturated at 1 μM, and the loading signal was 5- to 10-fold higher than that of the original SpyTag peptide at the highest concentration tested. The proteolytic stability of SpyTag003(D2) was qualitatively evaluated by MALDI-ToF MS. SpyTag003(D2) was stable past 24 hours in cell-spiked complete media (10% FBS) and up to 8 hours in normal mouse serum, indicating good stability for a linear peptide (FIG. 5A-5B). Proteolytic cleavage was frequently observed at or adjacent to the aspartic acid residue in SpyTag003(D2), which is consistent with predicted endoproteinase AspN activity at this position (Gasteiger, et al., A. Protein Identification and Analysis Tools on the ExPASy Server BT-The Proteomics Protocols Handbook; Walker, J. M., Ed.; Humana Press: Totowa, NJ, 2005; pp 571-607). Mindful that this residue forms the covalent isopeptide bond with all generations of SpyCatcher, further modification of the SpyTag003(D2) peptide were not pursued in this Example.

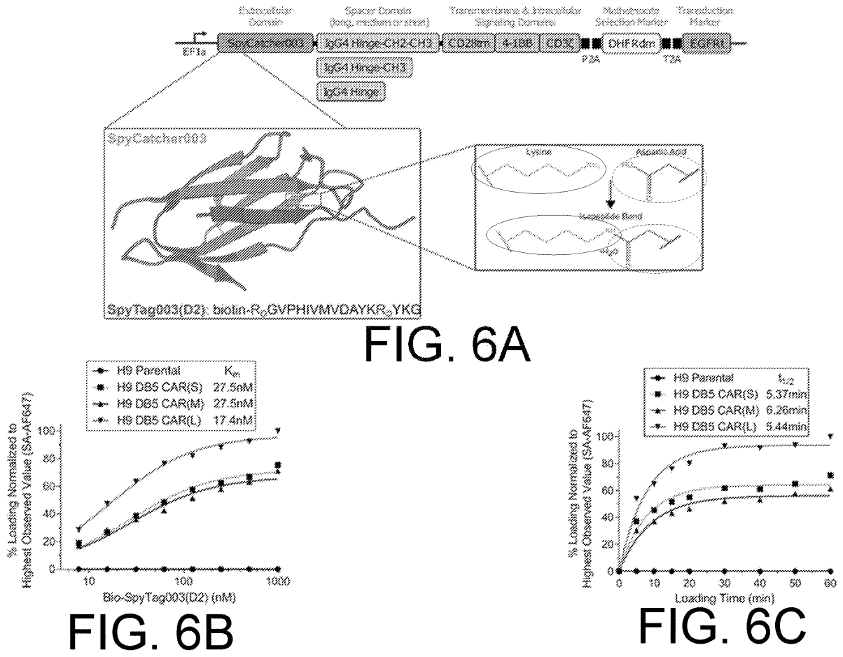

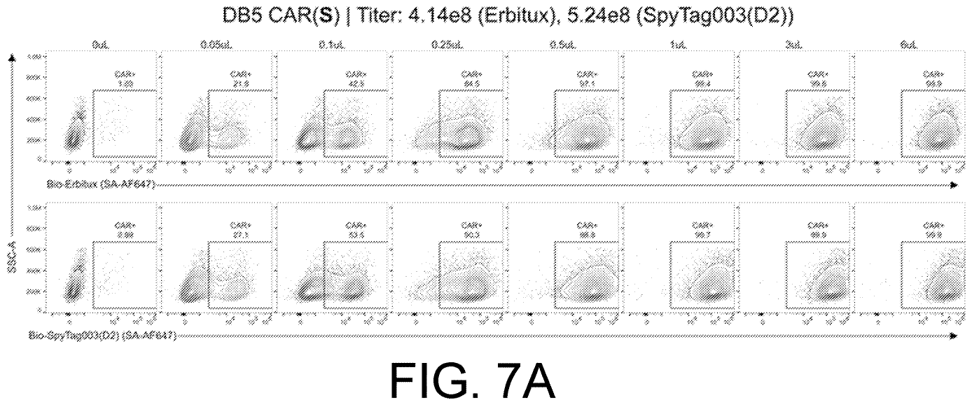

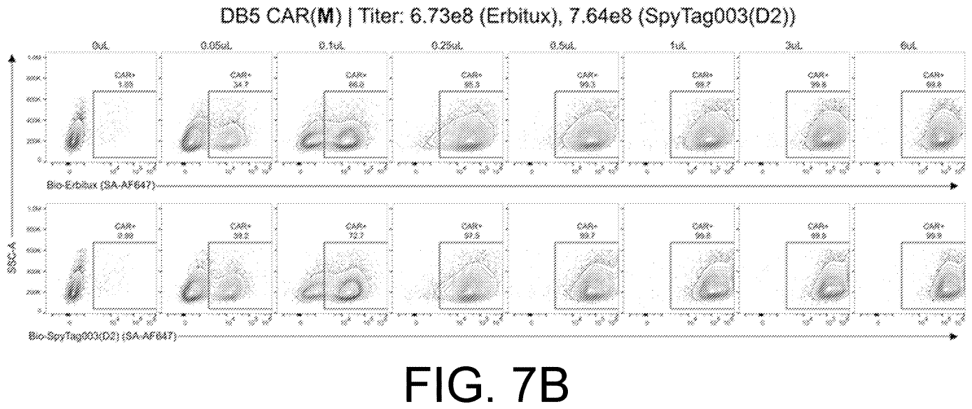

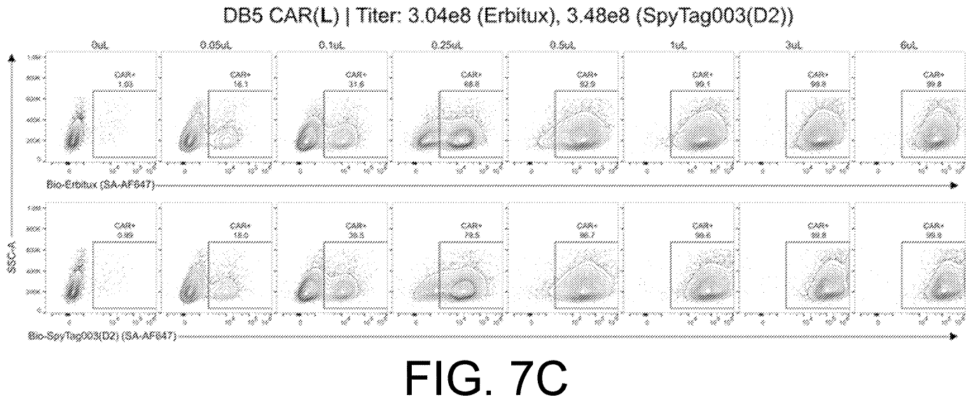

SpyCatcher003 (113 amino acids) was cloned into the three lentiviral CAR constructs with different spacers lengths as described before to make second-generation SpyCatcher003-41BB-CD3ζ CARs that would react even more efficiently with SpyTag003(D2) (FIG. 6A). These receptors are referred to as “DB5 CARs.” The resulting DB5 CAR lentiviruses were titered in H9 cells, and EGFRt and DB5 CAR expression were assayed in parallel by staining with biotinylated Erbitux antibody and SpyTag003(D2) peptide, respectively. SpyTag003(D2) staining of the DB5 CARs on H9 cells tracked similarly with Erbitux staining of EGFRt across all concentrations of lentivirus tested, even showing greater sensitivity than Erbitux for detecting transduced cells at lower lentivirus concentrations (FIG. 7A-7C). The lentiviruses also induced efficient construct expression, providing titers >10⁸ TU/mL regardless of the CAR spacer length.

To further characterize the reaction kinetics of SpyTag003(D2) with DB5 CARs, H9 DB5 CAR cells were incubated with various concentrations of SpyTag003(D2) and evaluated loading by flow cytometry. SpyTag003(D2) peptides loaded efficiently and selectively onto H9 cells expressing DB5 CARs, with robust loading occurring even below 10 nM (FIG. 6B) Fitting the loading data to a Michaelis-Menten model, Kₘ values ranged tightly from 20-30 nM regardless of the DB5 CAR spacer length. The time kinetics of loading at a sub-saturating concentration of peptide (50 nM) was tested, and the association half-time of SpyTag003(D2) to H9 DB5 CAR cells was fast at 5-6 min (FIG. 6C). These results demonstrate a significant improvement in loading kinetics compared to published universal CAR systems that rely on previously developed SpyCatcher-SpyTag chemistry.³⁸

Synthesis and Characterization of an αvβ6-Targeting Bifunctional Peptide

Peptides offer several advantages over antibodies as therapeutic targeting ligands for cancer. Their small size supports tumor penetration with low accumulation in off-target tissues and organs, their synthetic synthesis enables inexpensive production with high chemical diversity, they have low toxicity, and they can be readily modified with drugs or other peptides for multiplexed properties.⁴⁵

Accordingly, a bifunctional peptide intermediate for directing DB5 CAR function against a relevant cancer target was created. The integrin αvβ6 is upregulated in many solid cancers and its expression is associated with poor prognosis (Bandyopadhyay, et al., Current Drug Targets 2009, 10 (7), 645-652). As αvβ6 is not expressed in healthy adult epithelia, the integrin has become the focus of research in recent years aiming to develop targeting ligands for cancer imaging and therapeutic applications (Liu, et al., American J. of Nuclear Medicine and Molecular Imaging 2014, 4 (4), 333-345; Goodman, et al., Trends in Pharmacological Sciences 2012, 33 (7), 405-412). A20FMDV2, a 20-mer peptide derived from foot-and-mouth disease virus that binds αvβ6 with high affinity and specificity, is the successful culmination of much of this research (Slack, et al., Pharmacology 2016, 97 (3-4), 114-125). A20FMDV2 has been used to image and deliver drugs to αvβ6⁺ tumors in vivo (Saha, et al., The J. of Pathology 2010, 222 (1), 52-63; Moore, et al., Theranostics 2020, 10 (7), 2930-2942), and researchers have fused it to a CAR receptor for potent αvβ6-directed adoptive T-cell therapy (Whilding, et al., Molecular Therapy 2017, 25 (1), 259-273). For these reasons, an αvβ6-targeting bifunctional peptide that incorporates A20FMDV2 was constructed to validate the DB5 CAR system.

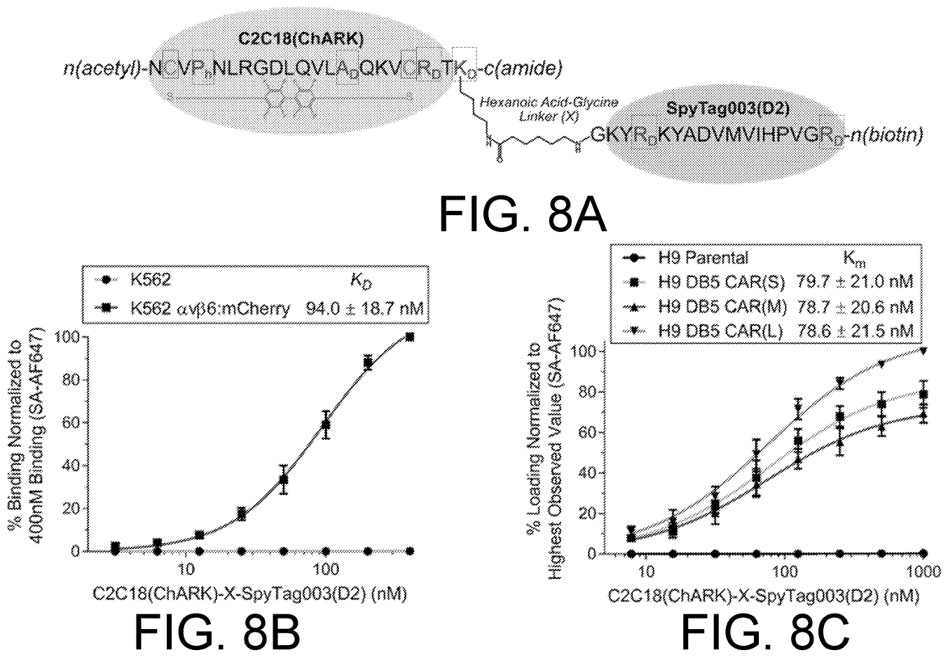

Previously, cysteine substituted A20FMDV2 variants cyclized by perfluoroarylation with decafluorobiphenyl (DFBP) were designed and modified with non-natural amino acids to increase the peptide's poor stability in serum (Cardle, et al., J. of Biological Chemistry 2021, 296). Therefore, to ensure the proteolytic stability of the peptide intermediate for in vitro and in vivo usage with DB5 CAR T-cells, the DFBP-cyclized C2C18 A20FMDV2 variant was used to synthesize the αvβ6-targeting arm of the bifunctional peptide. Hydroxyproline and D-alanine substitutions in between the cysteine linkages were incorporated, since these were found to increase the αvβ6 integrin specificity of the peptide, and D-arginine and D-lysine modifications were made to the C-terminus for added stability and to decrease potential immunogenicity. The resulting peptide, referred to as “C2C18(ChARK),” contained an acid-labile 4-methyltrityl (Mtt) group protecting the D-lysine side-chain, which was selectively deprotected for branched synthesis of the biotinylated SpyTag003(D2) arm to generated the final C2C18(ChARK)-X-SpyTag003(D2) bifunctional peptide (FIG. 8A) (Li, et al., The J. of Peptide Research 2002, 60 (5), 300-303). Hexanoic acid and glycine were included as a flexible linker (X) between the two peptide arms to promote proper peptide folding and prevent steric hindrance between αvβ6 and DB5 CAR interactions with the peptide (Karle, et al., Folding & Design 1997, 2 (4), 203-210).

To determine if the C2C18(ChARK)-X-SpyTag003(D2) peptide had bispecific properties, the peptide's ability to label both αvβ6⁺ cancer cells and DB5 CAR⁺ cells was evaluated. Using the matched erythroleukemia K562 and K562 αvβ6:mCherry cell lines, it was found that C2C18(ChARK)-X-SpyTag003(D2) exhibited high-affinity and selective binding for the K562 αvβ6:mCherry cells comparable to that of previously tested monofunctional C2C18 peptides, indicating functionality of the C2C18(ChARK) arm of the bifunctional peptide (FIG. 8B). Similarly, the bifunctional peptide loaded robustly onto H9 DB5 CAR cells with Kₘ values similar to those observed earlier with the monofunctional SpyTag003(D2) peptide (FIG. 8C). Having confirmed that C2C18(ChARK)-X-SpyTag003(D2) displays potent ambidextrous qualities, the peptide was tested in functional assays with CD4⁺ and CD8⁺ DB5 CAR T-cells.

In Vitro Efficacy of CD4⁺ DB5 CAR T-Cells Directed with Bifunctional Peptide

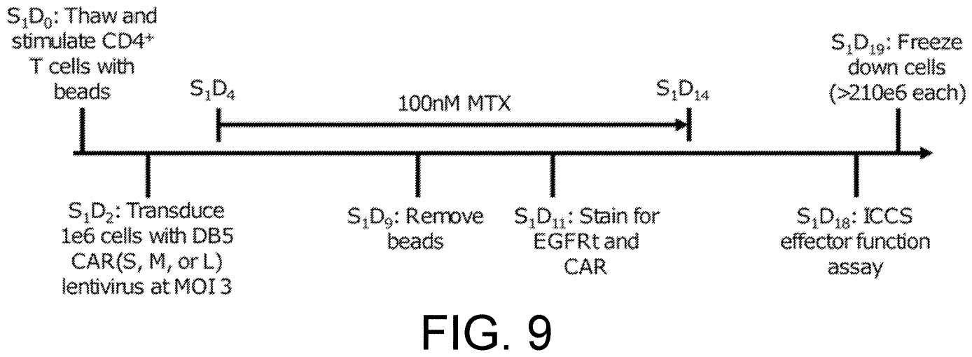

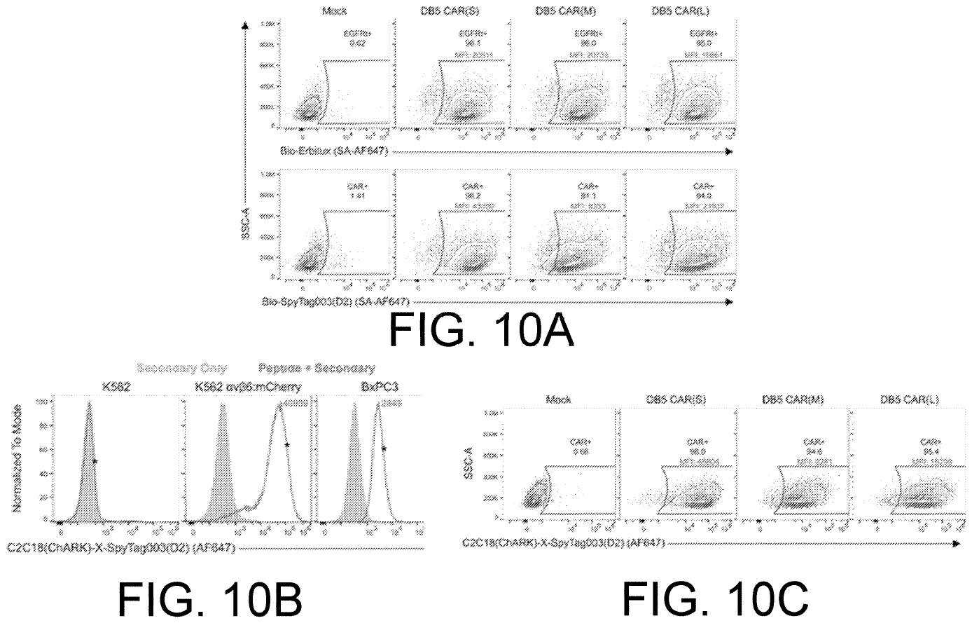

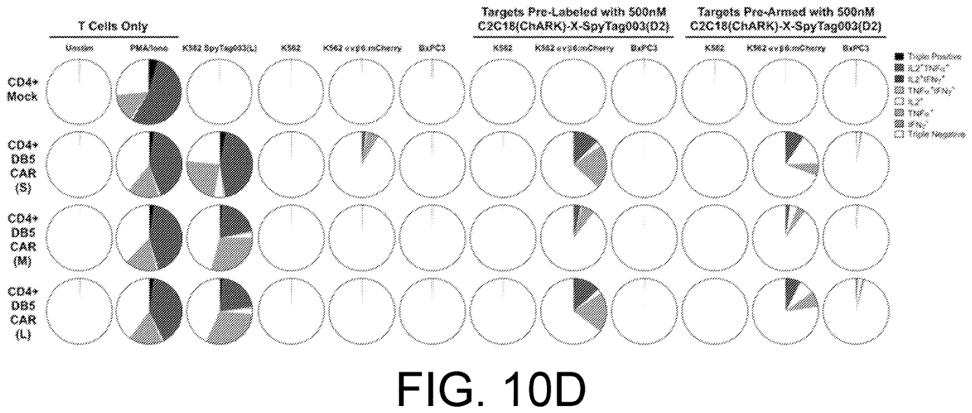

To demonstrate whether synthetic peptide intermediates can effectively guide DB5 CAR function, CD4⁺ DB5 CAR T-cells of all three spacer lengths were generated. CD4⁺ CAR T-cells were manufactured using a three-week stimulation bead outgrowth protocol, as summarized in FIG. 9, where S₁ denotes the bead stimulation and D_{#} signifies the number of days since the onset of stimulation. Transduced cells were enriched by methotrexate selection as previously described (Jonnalagadda, et al., Gene Therapy 2013, 20 (8), 853-860). After a week of methotrexate selection (S₁D₁₁), high transduction was confirmed by both Erbitux staining for EGFRt (>95%) and SpyTag003(D2) loading on DB5 CARs (>91%) (FIG. 10A). Interestingly, while EGFRt expression was near-identical across all three spacer lengths of the CARs, as indicated by median fluorescence intensity (MFI) values of Erbitux staining, SpyTag003(D2) loading on the CD4⁺ DB5 CAR T-cells varied depending on the CAR spacer length. The highest SpyTag003(D2) loading was observed on T-cells expressing the short spacer CAR, followed by the long spacer CAR, and then lastly the medium spacer CAR. As the DB5 CARs should be stoichiometrically translated and expressed with EGFRt, this data suggests that the spacer itself influences the ability of the SpyCatcher003 domain to react with SpyTag0003 D2 in solution. To bring transduction closer to 100%, cells were selected with methotrexate for another three days (10 days total) before being further expanded without selection for functional testing on S₁D₁₈.

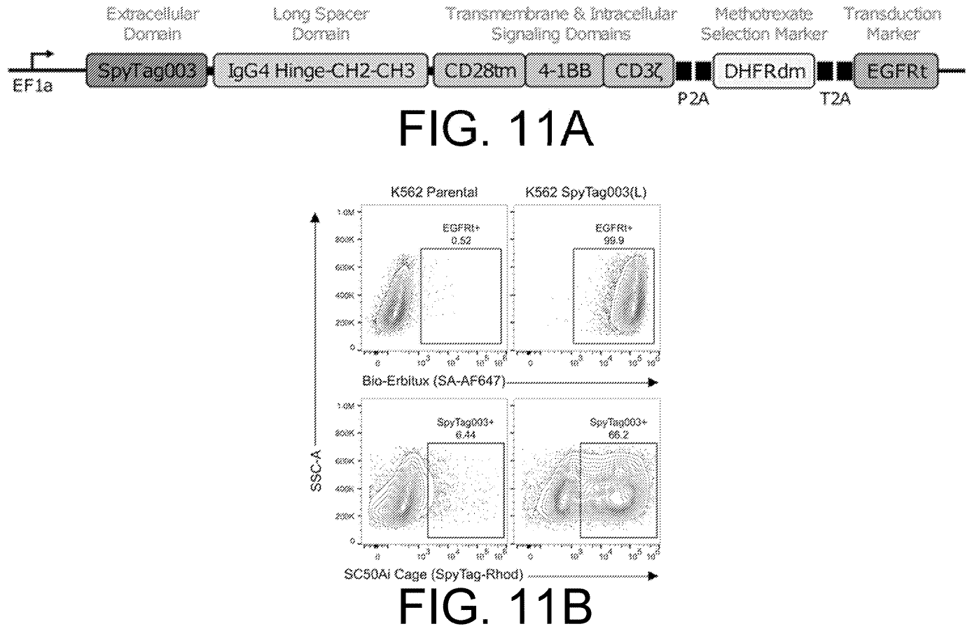

The capacity of these CD4⁺ DB5 CAR T-cells to produce cytokines against αvβ6⁺ target cells when directed by the C2C18(ChARK)-X-SpyTag003(D2) bifunctional peptide was evaluated. For target cells, the matched K562 and K562 αvβ6:mCherry cell lines as well as pancreatic ductal adenocarcinoma BxPC3 cells (which naturally express αvβ6 at more physiologically relevant levels) were used.⁵² K562 SpyTag003(L) cells that express SpyTag003 on their surface were also created to be used as a positive control target for the DB5 CAR T-cells (FIG. 11A-11B). Two orientations of guiding T-cell effector function with the peptide were explored. In one, target cells were pre-labeled with 500 nM bifunctional peptide for DB5 CAR-driven recognition of SpyTag003(D2) peptide presented on the target cell surface, and successful αvβ6-specific pre-labeling was confirmed by flow cytometry (FIG. 10B). As expected, BxPC3 cells were found to have >10-fold less expression of αvβ6 compared to K562 αvβ6:mCherry cells, making this a more stringent target model for the DB5 CAR system. In the other orientation, T-cells were pre-armed with 500 nM bifunctional peptide for peptide-driven recognition of αvβ6 on the target cell surface, and robust pre-arming (>94%) was confirmed by flow cytometry (FIG. 10C). Notably, the MFI of bifunctional peptide pre-arming on the DB5 CAR T-cells was spacer-dependent (short>long>medium), reaffirming the previous SpyTag003(D2) loading results on S₁D₁₁.

After pre-labeling and pre-arming with the bifunctional peptide, target cells and CD4⁺ T-cells were co-cultured together for 5 h and intracellular cytokine staining (ICCS) of IL2, TNFα, and IFNγ production was subsequently carried out to assess peptide-induced DB5 CAR T-cell activation (FIG. 10D). CD4⁺ DB5 CAR T-cells actively produced cytokines when co-cultured with K562 SpyTag003(L) cells but not when co-cultured with other bare target cells, with the exception of the short spacer CD4⁺ DB5 CAR T-cells that displayed a small amount of cytokine activity against bare K562 αvβ6:mCherry cells, validating that the DB5 CAR system mostly works as expected. Importantly, K562 αvβ6:mCherry cells pre-labeled with the C2C18(ChARK)-X-SpyTag003(D2) bifunctional peptide induced strong cytokine production in the DB5 CAR T-cells, whereas pre-labeled K562 cells did not promote such cytokine responses. Comparable results were found with pre-armed DB5 CAR T-cells co-cultured with K562 and K562 αvβ6:mCherry cells, demonstrating that the bifunctional peptide can effectively steer DB5 CAR T-cell responses against K562 αvβ6:mCherry regardless of its orientation.

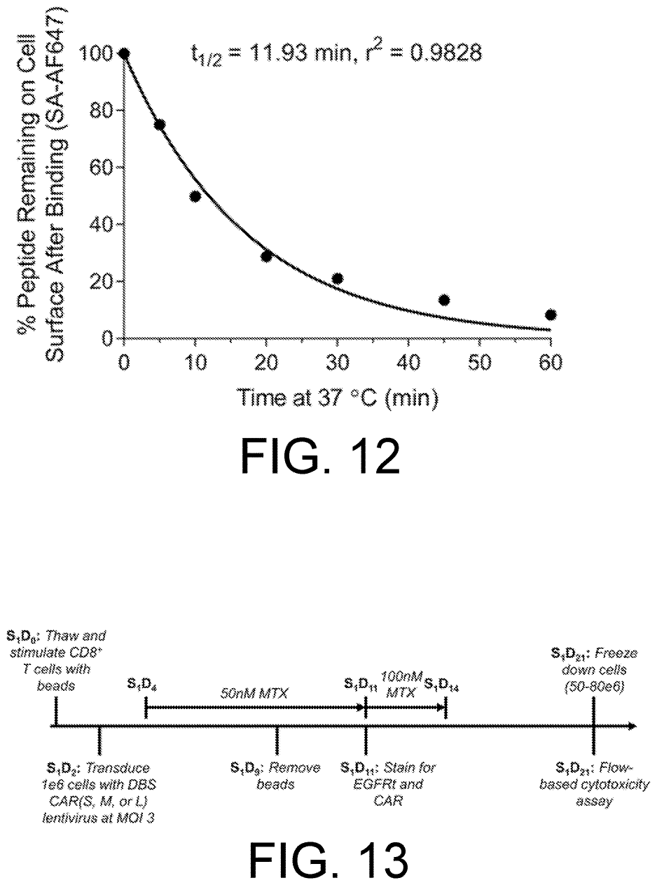

Differences, however, were observed in the efficacy of bifunctional peptide pre-labeling and pre-arming with the BxPC3 cells. Whereas pre-labeled BxPC3 cells did not induce cytokine production in DB5 CAR T-cells, modest cytokine responses against BxPC3 cells were observed when DB5 CAR T-cells were pre-armed with the bifunctional peptide. This may suggest that pre-labeled BxPC3 cells internalize the peptide too quickly for unarmed DB5 CAR T-cell recognition, which is possible given the reported fast internalization kinetics of A20FMDV2-bound αvβ6 (t_{1/2}=1.5 min).⁴⁹ Indeed, it was found that pre-labeled BxPC3 cells internalized the bifunctional peptide with a half-life of 12 min, meaning <5% of starting bifunctional peptide remains on the BxPC3 cell surface after just a 60-min co-incubation with T-cells at 37 °C (FIG. 12). Nevertheless, the bifunctional peptide is expected to both label tumors and arm T-cells in vivo after intravenous injection, and T-cell arming should outlast tumor labeling given the relatively slower turnover rate of CAR receptors (t_{1/2}=8-12 h) (Roybal, et al., Cell 2016, 164 (4), 770-779; Choe, et al., Science Translational Medicine 2021, 13 (591), eabe7378), so the observed cytokine production of pre-armed DB5 T-cells against BxPC3 cells would suggest in vivo translation is possible.

Of the different spacer lengths tested, the long spacer DB5 CAR appeared to function the best when armed with this bifunctional peptide intermediate, eliciting the greatest cytokine response against BxPC3 cells. Interestingly, the medium spacer DB5 CAR failed to initiate cytokine responses against BxPC3 cells when armed with bifunctional peptide and also induced the least cytokine responses against K562 αvβ6:mCherry regardless of pre-arming or pre-labeling. The stunted cytokine activity of the medium spacer DB5 CAR is consistent with the low SpyTag003(D2) loading/arming previously observed with this spacer length compared to the short and long spacer DB5 CARs (FIGS. 10A, 10C), suggesting that the medium spacer length does not display fully functional SpyCatcher003 on the T-cell surface.

In Vitro Efficacy of CD8⁺ DB5 CAR T-Cells Directed with Bifunctional Peptide

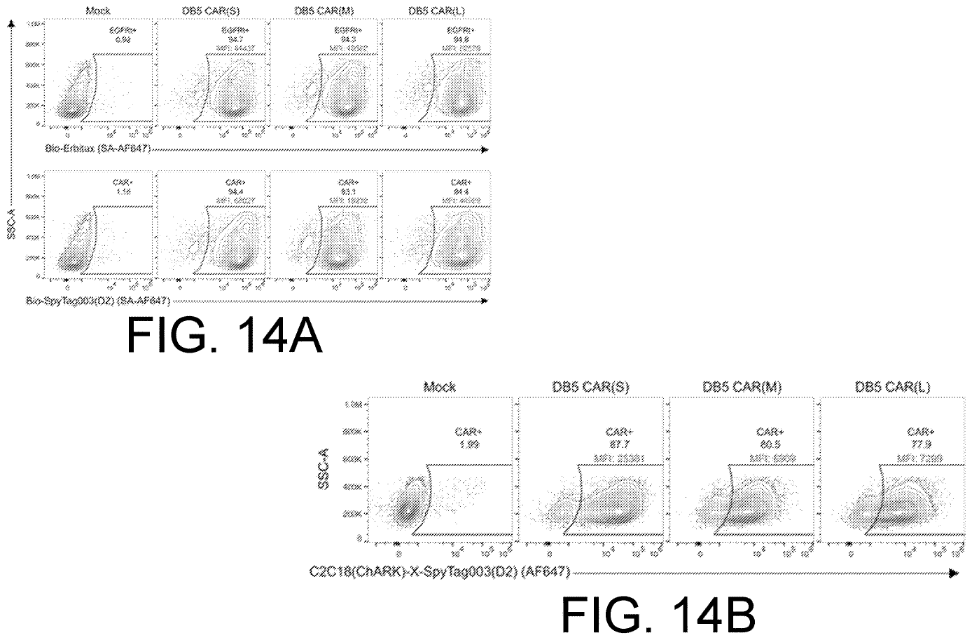

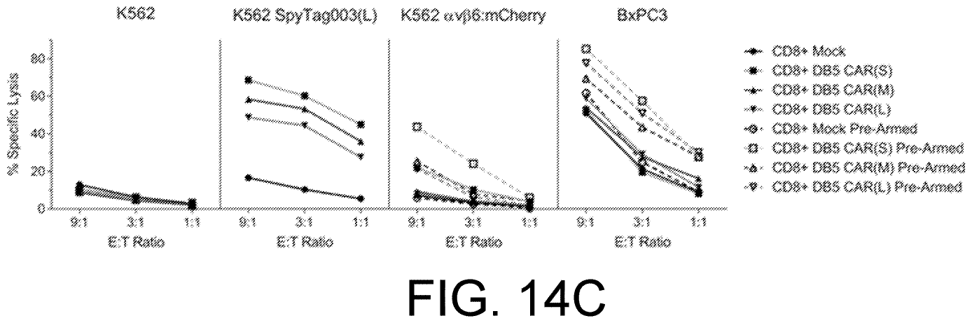

CD8⁺ DB5 CAR T-cells of all three spacer lengths were generated for peptide-directed cytotoxicity studies. CD8⁺ CAR T-cells were sourced from the same donor as the CD4⁺ T-cells used previously, and cell manufacturing comprised a similar three-week stimulation bead outgrowth protocol to that used before, except methotrexate concentrations for selection were reduced given the greater sensitivity of CD8⁺ T-cells to the drug (FIG. 13). After a week of methotrexate selection (S₁D₁₁), high transduction was confirmed by both Erbitux staining for EGFRt (>94%) and SpyTag003(D2) loading on DB5 CARs (>93%) (FIG. 14A). Consistent with the CD4⁺ DB5 CAR T-cells, while the MFI value of EGFRt expression was near-identical across all three spacer lengths of the CARs, SpyTag003(D2) loading on the CD8⁺ DB5 CAR T-cells was the highest on T-cells expressing the short spacer CAR, followed by the long spacer CAR, and then lastly the medium spacer CAR. These data further support that the medium spacer length struggles to properly present SpyCatcher003 on the cell surface.

Next, the capacity of these CD8⁺ DB5 CAR T-cells to kill αvβ6⁺ target cells when directed by the C2C18(ChARK)-X-SpyTag003(D2) bifunctional peptide was determined. Based on the prior results of pre-arming CD4⁺ T-cells versus pre-labeling target cells in FIG. 10C, only CD8⁺ T-cells with 500 nM bifunctional peptide were pre-armed for the cytotoxicity assay since that was found to be most effective in generating responses against BxPC3 cells. Moderate pre-arming of CD8⁺ DB5 CAR T-cells (>77%) for all space lengths was confirmed by flow cytometry (FIG. 14B). The MFI of bifunctional peptide pre-arming on the DB5 CAR T-cells was spacer-dependent (short>long>medium), tracking with both the SpyTag003(D2) loading results on S₁D₁₁ and the pre-arming results in the CD4⁺ DB5 CAR T-cell ICCS study. For target cells, K562, K562 αvβ6:mCherry, K562 SpyTag003(L), and BxPC3 cells were pre-labeled with a CellTrace dye to distinguish them from effector CD8⁺ T-cells by flow cytometry. Effector CD8⁺ T-cells and target cells were co-cultured together at different effector-to-target (E:T) ratios for 18 h before staining cells with a viability dye to assess killing of the CellTrace⁺ target cells by flow cytometry. An 18-h co-culture was used, instead of a 4-h co-culture that is commonly used for chromium release assays, since other groups have reported the need for longer co-culture times to adequately measure T-cell-mediated cytotoxicity of tumor cells with this more direct, non-radioactive assay (Nelson, et al., Oncolmmunology 2019, 8 (8), 1-10).

As shown in FIG. 14C, CD8⁺ DB5 CAR T-cells potently lysed K562 SpyTag003(L) cells over a mock T-cell control, demonstrating thaT-cell surface-displayed SpyTag003 mediates robust DB5 CAR activity. The magnitude of K562 SpyTag003(L) lysis also discernibly increased as the spacer length of CD8⁺ DB5 CAR T-cells decreased, which is expected given that SpyTag003 is spaced distally from the cell surface in this target cell line and thus shorter DB5 CARs should form a narrower synapse with these cells for increased CD45 phosphatase exclusion and T-cell activation (Xiao, et al., Science Immunology 2022, 7 (74), eabl3995). Neither CD8⁺ mock nor DB5 CAR T-cells actively lysed K562 cells, regardless of bifunctional peptide arming, and unarmed CD8⁺ DB5 CAR T-cells did not lyse K562 αvβ6:mCherry and BxPC3 targets over the mock T-cell controls, with the exception of the unarmed short spacer CD8⁺ DB5 CAR T-cells that induced some lysis of K562 αvβ6:mCherry cells. This unusual activity of the unarmed shorter spacer DB5 CAR T-cells against K562 αv6:mCherry cells was also observed previously in the CD4⁺ T-cell ICCS study, indicating that this is a real response.

Analyzing pre-armed CD8⁺ DB5 CAR T-cell activity, it was observed that peptide-directed lysis of K562 αvβ6:mCherry and BxPC3 cells for all DB5 CAR spacer lengths that titrated with the E:T ratio. The short spacer DB5 CAR elicited the greatest lysis of K562 αvβ6:mCherry and BxPC3 cells by CD8⁺ T-cells when pre-armed with bifunctional peptide, although the background lysis of K562 αvβ6:mCherry cells with unarmed short spacer DB5 CAR makes the K562 αvβ6:mCherry cytotoxicity results difficult to interpret. The pre-armed long spacer DB5 CAR was only slightly inferior at lysing BxPC3 cells than the short spacer DB5 CAR despite having significantly less pre-arming as indicated in FIG. 14B, suggesting that the long spacer DB5 CAR may be more potent with the bifunctional peptide. The pre-armed medium spacer DB5 CAR induced the least lysis of BxPC3 cells, consistent with the previous CD4⁺ T-cell ICCS results and SpyTag003(D2) loading observations. Altogether, the functional assays with CD4⁺ and CD8⁺ T-cells demonstrate that the C2C18(ChARK)-X-SpyTag003(D2) peptide can effectively direct DB5 CAR T-cell activity against αvβ6⁺ cancer cell targets, especially when covalently armed onto T-cells, and that the DB5 CAR spacer length heavily influences T-cell responses with the bifunctional peptide intermediate.

### Synthesis and Characterization of a Heterobifunctional Aptamer-Peptide Chimera

Besides peptides, other types of synthetic targeting intermediates that could interface with the DB5 CAR system for cancer targeting were also evaluated. DNA aptamers are single-stranded oligonucleotides that fold into sequence-specific secondary structures capable of recognizing cellular and protein targets with high affinity (Zhou, et al., Nature Reviews Drug Discovery 2017, 16 (6), 440). Importantly, aptamers are small (10-30 kDa), non-toxic, and amenable to modification with drugs or other peptides for multiplexed properties, highlighting their potential for cancer recognition and treatment (Bouchard, et al., Annual Review of Pharmacology and Toxicology 2010, 50 (1), 237-257; Tan, et al., ACS Applied Materials & Interfaces 2021, 13 (8), 9436-9444). A high-affinity DNA aptamer that selectively binds lymphoid-derived leukemia and lymphoma cells (e.g., JurkaT-cells) over myeloid-derived counterparts (e.g., K562 cells) and healthy immune cells (unpublished) was used to create a synthetic heterobifunctional aptamer-peptide chimera for directing DB5 CAR T-cell function.

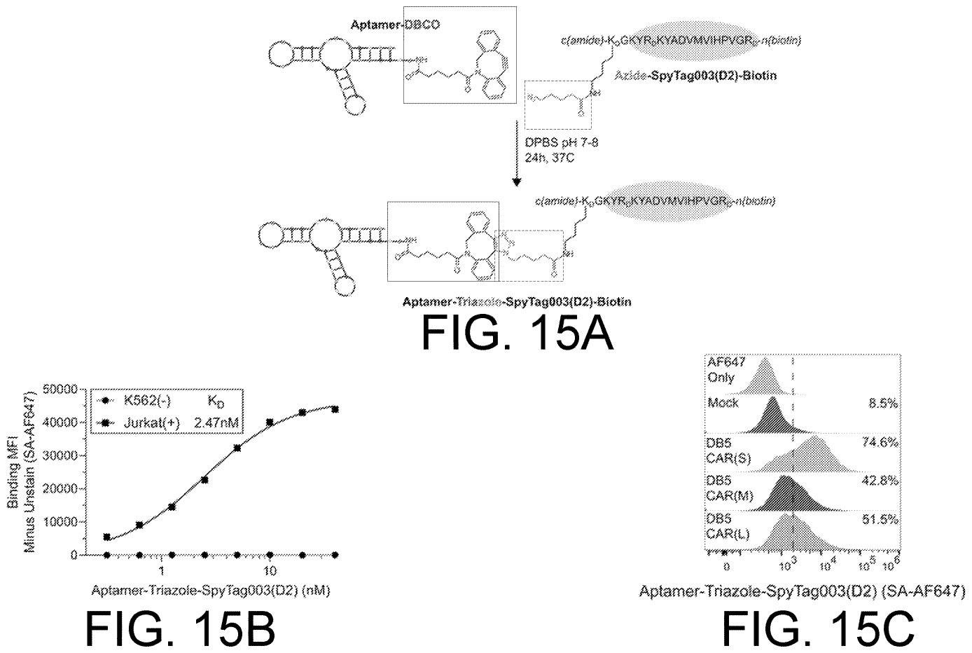

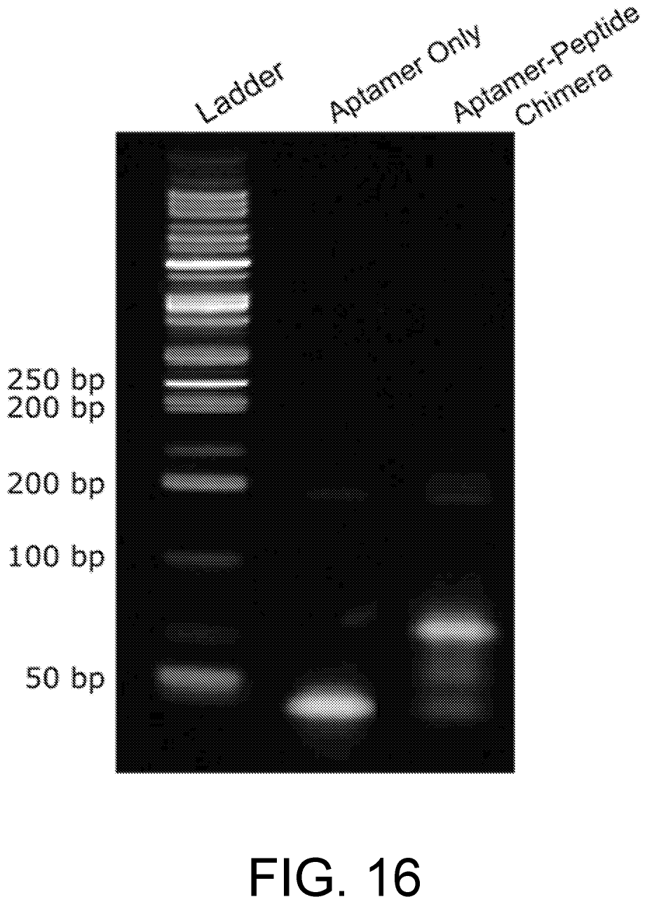

To construct the aptamer-peptide chimera, strain-promoted azide-alkyne cycloaddition (SPAAC) was used, which is a copper-free click reaction that occurs spontaneously with high yield under mild aqueous conditions and is orthogonal to other biochemical reactions (Yoon, et al., Advanced Materials 2022, 34 (10), 2107192). Biotinylated SpyTag003(D2) was first synthesized with a C-terminal D-lysine that contained an acid-labile Mtt group protecting the side-chain amino group, which was selectively deprotected and coupled with 5-azidopenatoic acid to produce azide-SpyTag003(D2)-biotin. The aptamer was then commercially manufactured with a 5′ dibenzocyclooctyne (DBCO) modification and reacted with azide-containing peptide to form a triazole bridge, resulting in the chimera called Aptamer-Triazole-SpyTag003(D2)-biotin. (FIG. 15A). Denaturing urea polyacrylamide gel electrophoresis (urea-PAGE) confirmed successful conjugation of the aptamer to the peptide with >80% yield, as demonstrated by an upward shift in the DNA band compared to an unconjugated aptamer control (FIG. 16). The aptamer-peptide chimera was subsequently purified by ethanol precipitation to remove excess unreacted peptide before proceeding to characterization studies.

To determine if the aptamer-peptide chimera had bifunctional properties, binding of the chimera to T-leukemia JurkaT-cells (aptamer target positive) and myeloid leukemia K562 cells (aptamer target negative) was evaluated. Aptamer-Triazole-SpyTag003(D2)-biotin was found to selectively bound JurkaT-cells with high affinity, indicating functionality of the aptamer arm of the chimera (FIG. 15B). Furthermore, as streptavidin Alexa Fluor 647 was used as a secondary stain to measure binding, this data also demonstrates successful conjugation of the aptamer to SpyTag003(D2)-biotin, since unconjugated aptamer does not have biotin for streptavidin recognition. Loading of the chimera on primary CD8⁺ mock and DB5 CAR T-cells was evaluated, as characterized in FIGS. 14A-14C. Aptamer-Triazole-SpyTag003(D2)-biotin loaded selectively on CD8⁺ DB5 CAR T-cells of all spacer lengths over the mock control, showing functionality of the peptide arm of the chimera (FIG. 15C). Chimera loading was highest on CD8⁺ T-cells expressing the short spacer CAR, followed by the long spacer CAR, and then lastly the medium spacer CAR, which is consistent with the trend of C2C18(ChARK)-X-SpyTag003(D2) loading on these cells in FIG. 14B. While some chimera loading was observed on mock T-cells, this can be attributed to aptamer binding since the aptamer is known to have low binding to healthy lymphocytes. Comparatively speaking, the aptamer exhibits much greater binding to lymphocytic cancer cells, so off-tumor DB5 activity that would result in unwanted killing of healthy immune cells or fratricide of the CAR T-cell product was not anticipated. Taken together, the Aptamer-Triazole-SpyTag003(D2)-biotin chimera displays potent heterobifunctional qualities.

### DISCUSSION

CAR T-cell therapy has demonstrated great potential to treat cancer but relapse due to antigen escape and toxicities have limited the therapy's broader clinical impact. Universal CAR systems that decouple antigen targeting from the CAR represent a promising solution to these problems. In these approaches, externally supplemented targeting intermediates are used to bridge CAR T-cell activity with antigen targets, giving researchers and clinicians greater control over the therapy's direction and outcome. A panel of intermediate ligands can be tailored to a patient's heterogenous cancer antigen profile for comprehensive therapy that requires only one CAR T-cell product, and intermediates can be further adapted over treatment to counter a tumor's dynamic plasticity that promotes antigen escape. As the intermediates effectively control antigen presentation and can be cleared from circulation quickly, the concentration and frequency of intermediate dosage can also be precisely regulated to mitigate side effects associated with therapy.

Most universal CAR systems to-date rely on transient noncovalent recognition of tagged targeting intermediates for function, which is unstable and can diminish CAR T-cell activity. For this reason, covalent SpyCatcher-SpyTag chemistry has become an attractive method for directing universal CAR T-cells function, since T-cells can be stably armed with intermediates for prolonged targeting. Indeed, Minutolo et al. showed that covalent recognition of SpyTag-labeled intermediates by SpyCatcher CAR T-cells provided better T-cell arming and activation than affinity-based recognition of the same intermediates labeled with a non-reactive SpyTag mutant.³⁸ Despite this, when compared to conventional CAR T-cells, their armed SpyCatcher CAR T-cells displayed less overall potency and also lost sensitivity when target antigen expression was low. These results are potentially attributed to the slow kinetics of the SpyCatcher-SpyTag reaction that requires micromolar concentrations of each partner, underscoring the need for more efficient covalent universal CAR systems that can be feasibly translated. Additionally, despite the versatility of these approaches, published universal CAR systems have not capitalized on the different classes of ligand intermediates available for cancer targeting, such as synthetic peptides and aptamers, and instead have relegated their usage to well-established antibody, protein, and small molecule intermediates. Given the unique advantages of synthetic peptides and aptamers, there is an opportunity for expanding the toolkit of intermediates used with universal CAR systems.

This Example provides a cyborg universal CAR system that uses accelerated SpyCatcher003-SpyTag003 chemistry to covalently modify T-cell effector function with synthetic targeting materials. These DB5 CARs, and their cognate peptide SpyTag003 exhibit enhanced arming and reaction kinetics compared to CARs that use original SpyCatcher-SpyTag chemistry. To redirect DB5 CAR T-cell effector function against target cancer antigens, a branched peptide intermediate containing both SpyTag003 and a serum-stabilized A20FMDV2 peptide (for high-affinity targeting of αvβ6⁺ tumor cells) was synthesized. The branched peptide displayed robust bispecific adaptor properties, capable of selectively recognizing both αvβ6⁺ cancer cells and DB5 CAR-expressing cells. This Example demonstrates the bifunctional peptide's ability to induce CD4⁺ DB5 CAR T-cell cytokine production and CD8⁺ DB5 CAR T-cell killing in vitro when pre-labeled on αvβ6⁺ target cells and pre-armed on DB5 CAR T-cells. As shown herein, these responses are dependent on the extracellular spacing of the DB5 CAR. Diversifying the toolkit of synthetic intermediates that can be used with the DB5 CAR system, this Example also presents a heterobifunctional aptamer-peptide chimera capable of selectively recognizing both cancer cells and DB5 CAR-expressing cells.

There are many exciting opportunities afforded by these cyborg CAR T-cells. Besides using different concentrations of synthetic targeting materials to titrate DB5 CAR T-cell activation, different generations of SpyTag may be used to a similar effect. SpyTag, SpyTag002, and SpyTag003 react with SpyCatcher003 with rate constants spanning a 20-fold range of values, suggesting that they can be interchanged in the bifunctional peptide to fine-tune the strength of cyborg CAR T-cell activation. Moreover, synthetic materials can be modified to incorporate targeting ligands that permit T-cell accumulation in solid tumors, and synthetic materials can incorporate multiple SpyTag peptides that can group CAR on the surface of a T-cell to potentially augment cytokine release and cell killing. Additionally, DB5 CARs can be partnered with other covalent systems that are orthogonal to SpyCatcher-SpyTag chemistry for logic-gated functions. Split from the same adhesin in Streptococcus pneumoniae, SnoopCatcher-SnoopTag and DogCatcher-DogTag are protein-peptide pairs that spontaneously form isopeptide bonds with each other and show no cross-reaction to SpyCatcher and SpyTag (Veggiani, PNAS 2016, 113 (5), 1202-1207; Keeble, et al., Cell Chemical Biology 2022, 29 (2), 339-350.e10). Orthogonal SpyCatcher-SpyTag and SnoopCatcher-Snoop Tag or DogCatcher-DogTag chemistries can thus be fashioned together in trans-signaling CAR strategies or in synNotch receptor circuits for AND-gated T-cell activation that utilizes intermediate-guided dual antigen recognition (Lanitis, et al., Cancer Immunology Research 2013, 1 (1), 43-53).

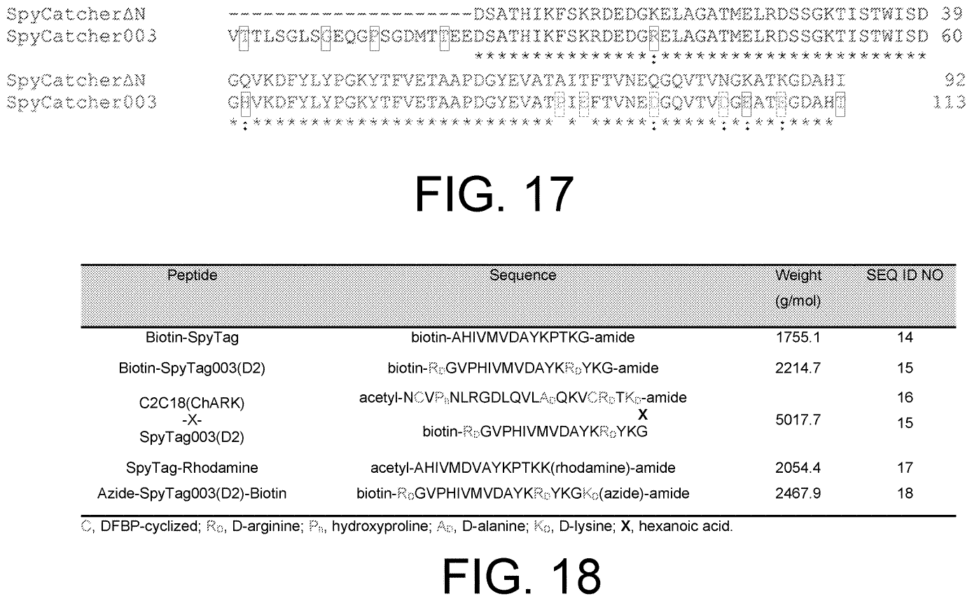

As for SpyCatcher003, it was previously shown that N-terminal truncation of the SpyCatcher protein can lower the antibody responses it induces in immunocompetent mice without affecting its reaction with SpyTag (Liu, et al., Scientific Reports 2014, 4 (1), 7266). Given that the mutations made from SpyCatcher002 to SpyCatcher003 are all localized at the protein's C-terminus (FIG. 17) (Keeble, PNAS 2019, 116 (52), 26523-26533), and that the N-terminus of SpyCatcher is known to not have any direct interaction with SpyTag (Li, et al., J. of Molecular Biology 2014, 426 (2), 309-317), it is anticipated that an N-terminal truncated version of SpyCatcher003 can be swapped into the DB5 CARs to reduce immunogenicity without sacrificing the augmented reaction kinetics of the system.

In summary, this Example demonstrates the potential to utilize synthetic materials to arm/target a universal CAR system via efficient SpyCatcher003-SpyTag003 chemistry for directing T-cell responses against cancer cells in vitro.

### Materials and Methods

Cloning of lentiviral constructs and lentivirus production. DNA fragments with a Kozak sequence and an open reading frame encoding a GM-CSF signal peptide followed by SpyCatcher, SpyCatcher003, or SpyTag003 were synthesized by GeneArt and amplified by PCR prior to cloning. The three epHIV7.2 lentiviral vectors encoding scFv-spacer-CD28tm-41BB-CD3ζ-P2A-DHFRdm-T2A-EGFRt with short (IgG4 hinge), medium (IgG4 hinge-CH3), and long (IgG4 hinge-CH2-CH3) extracellular spacers were a gift from the Jensen Lab (Seattle Children's Research Institute). DNA fragments and lentiviral vectors were digested with Nhel and Rsrll restriction enzymes (NEB) to create inserts and scFv-excised backbones, respectively, that were subsequently gel purified (QIAGEN) and ligated with T4 DNA ligase (NEB). DH10B and Stb12 chemically competent E. coli (Thermo Fisher) were transformed with ligated products and kanamycin-selected colonies were screened by PCR for correct insert length. Correct cloning was verified by Sanger sequencing (GENEWIZ) of miniprep DNA (QIAGEN) before transfection-grade plasmid DNA was prepared by maxiprep (MACHEREY-NAGEL).

HEK 293T-cells were purchased from ATCC and used before passage 20. For each lentivirus production run, HEK 293T-cells were seeded 24 h prior to transfection in twenty 10 cm plates at 3×10⁶ cells per plate in 10 mL DMEM with high-glucose, L-glutamine, and sodium pyruvate (Life Tech) supplemented with 10% gamma-irradiated FBS (Life Tech) and 1× penicillin-streptomycin (Life Tech). The next day, half of the plating media (5 mL) was removed from the cells and each plate was transfected with 15 μL BioT transfection reagent (Bioland Scientific) mixed with 150 μL serum-free DMEM containing pMDL-RRE (2.9 μg), pRSV-Rev (1.1 μg), pVSV-G (1.6 μg), and transgene lentiviral vectors (4.5 μg). After 24 h, 5 mL complete DMEM media was added to the plates to make the supernatant 10 mL total and virus-containing supernatant was collected and replaced with 5 mL fresh media at 48 and 72 h post-transfection. At 96 h post-transfection, the last of the virus-containing supernatant was collected (400 mL total for twenty 10 cm plates) and cell debris was removed by 0.22 μm filtration. Virus was pelleted in two batches by ultracentrifugation at 18,500 rpm (58,486×g) for 2 h at 4 °C in a Beckman Coulter Optima L-100XP Ultracentrifuge using a SW 32 Ti rotor and 38.5 mL open-top tubes (Beckman Coulter). Pellets from both batches were resuspended and combined in 12 mL HBSS before being pelleted again by ultracentrifugation at 19,500 rpm (65,2020×g) for 2 h at 4 °C using a SW 41 Ti rotor and 13.2 mL open-top tubes (Beckman Coulter). The resulting viral pellet was resuspended in 200 μL HBSS and stored at −80 °C until further use.

### Peptide Synthesis, Purification, and Cyclization

Sequences of synthesized peptides are provided in the accompanying sequence listing. Materials for peptide synthesis are as previously described (Cardle, et al., J. of Biological Chemistry 2021, 296), with the addition of Fmoc-D-Lys(Mtt)-OH, Fmoc-Lys(Mtt)-OH, Fmoc-ε-Ahx-OH, acetic anhydride, pyridine, NHS-rhodamine, and 5-azidopentanoic acid purchased from Alfa Aesar (Haverhill, MA), Novabiochem (Darmstadt, Germany), AAPPTec (Louisville, Kentucky), EMD Millipore (Burlington, MA), Thermo Fisher (Waltham, MA), and BroadPharm (San Diego, CA). Peptide synthesis was done as previously described in Cardle et al., with the addition of peptide end-capping and Lys(Mtt) deprotection steps for the synthesis of branched peptides or addition of C-terminal rhodamine or 5-azidopentanoic acid. After synthesis of the first peptide arm, on-resin acetylation of the peptide N-terminus was carried out twice in 10 mL 3:2:1 (v/v) DCM:pyridine:acetic anhydride for 1 h at room temperature with end-over-end mixing. Resin with peptide was then washed 3 times with DCM and a Kaiser Test was used to qualitatively check N-acetylation as previously described (Coin, et al., Protocol Exchange 2007, 10). The Mtt-protecting group on Lys(Mtt) was removed by repeatedly incubating the resin with N-acetylated or N-biotinylated peptide in 10 mL 2% TFA in DCM (v/v) for 15 min at room temperature with end-over-end mixing until the deprotection solution turned from yellow-orange to clear (5-10 times). Resin was then washed 3 times with each DCM, DMF, and methanol before successful Mtt-deprotection was qualitatively confirmed by Kaiser Test. For further synthesis of branched peptides or on-resin coupling of 5-azidopentanoic acid, the resin with N-acetylated or N-biotinylated and Mtt-deprotected peptide was swelled in 50:50 (v/v) DMF:DCM for 20 min prior to further synthesis on a Liberty Blue HT12 automated microwave peptide synthesizer (CEM, Matthews, NC). For on-resin coupling of NHS-rhodamine, resin with N-acetylated and Mtt-deprotected peptide was swelled in DCM and then exchanged to 5 mL DCM with 2 molar equivalents NHS-rhodamine, 3 molar equivalents EDC, and 8 molar equivalents of triethylamine. After an overnight incubation at room temperature with end-over-end mixing, the resin with rhodamine-coupled peptide was washed 3 times in DCM before cleavage. All peptides were cleaved and ether precipitated as previously described,⁵³ with the exception of azide-SpyTag003(D2)-biotin that was cleaved in 92.5:2.5:2.5:2.5 TFA:triisopropylsilane:H₂O:thioanisol to limit azide to amine reduction (Schneggenburger, et al., J. of Peptide Science 2010, 16 (1), 10-14).

Peptides were purified by reverse-phase HPLC (Agilent 1260 Infinity, Santa Clara, CA) using a ZORBAX 300SB-C18 semi-preparative column (Agilent). For SpyTag peptides, a flow rate of 5 mL/min and a 30-60% or 25-65% 8-min linear solvent gradient of ACN in H₂O with 0.1% TFA were used for purification by monitoring 280 nm absorbance. For bifunctional peptides, purification conditions were similar but required a shallower and longer 30-55% 12-min linear solvent gradient. Bifunctional peptides were cyclized with DFBP as previously described.⁵³ Molecular weights of peptides were screened by MALDI-ToF MS (Bruker AutoFlexll, Billerica, MA) multiple times throughout the production process and were consistently within 1-2 g/mol of expected values.

### Cell Line Culture and T-Cell Isolation

The H9, K562, and BxPC3 cell lines were purchased from ATCC. The K562 αvβ6:mCherry cell line was a gift from A. Olshefsky (Pun and King Labs, University of Washington) and were generated as previously described.⁵³ The K562 SpyTag003(L) cell line was generated by transduction of 10⁶ K562 cells with lentivirus (3.26e8 TU/mL) encoding a SpyTag003 long-spacer CAR at a multiplicity of infection (MOI) of 3 with 5 μg/mL polybrene (EMD Millipore). The H9 cell lines expressing SpyCatcher and DB5 CARs of different spacer lengths were generated in lentivirus titering studies described below. All the above cell lines were cultured in complete RPMI comprised of RPMI 1640 medium with L-glutamine (Corning) supplemented with 10% FBS. Human peripheral blood mononuclear cells (PBMCs) were isolated from TRIMA LRS chambers (Bloodworks Northwest) using Ficoll-Paque density gradient centrifugation (GE). CD4⁺ and CD8⁺ T-cells were positivity selected in sequence from PBMCs by magnetic-activated cell sorting (MACS) using CD4 and CD8 Microbeads (Miltenyi) according to the manufacturer's instructions and were banked for later CAR T-cell production.

### Extracellular Flow Cytometry Binding Studies

Peptide stocks were prepared in H₂O at 5 mM and the exact concentration of biotinylated stocks was measured using a QuantTag Biotin Quantification Kit (Vector Labs). SC50Ai SpyCatcher nanocages were a kind gift from the Baker Lab (University of Washington) and were used at a 1:75 dilution (16.67 nM). Biotinylated Erbitux antibody was a kind from the Jensen Lab (Seattle Children's Therapeutics) and was used at a 1:1000 dilution. Prior to binding, cells were pre-stained with 1:500 Zombie Violet (BioLegend) in 100 μL DPBS (Gibco) per 10⁶ cells for 15 min at room temperature for dead cell discrimination. Cells were then washed with DPBS 1% BSA (Miltenyi) to neutralize remaining Zombie Violet and plated in a U-bottom black 96-well plate at 2×10⁵ cells per well. Primary staining (100 μL) was carried out under different conditions depending on the assay. For EGFRt staining, cells were stained with antibody diluted in DPBS 1% BSA for 20-30 min at room temperature. For αvβ6-binding experiments, cells were stained with peptides diluted in DPBS with calcium and magnesium (Corning) for 20 min at 4 °C. For loading via covalent SpyCatcher-SpyTag chemistry, cells were stained with peptides or nanocages diluted in DPBS for 30-60 min at room temperature. After primary staining, cells were washed twice with 200 μL appropriate buffer and then stained with the appropriate secondary (100 μL). For cells labeled with biotinylated ligands, cells were stained with streptavidin Alexa Fluor 647 diluted 1:500 in DPBS with calcium and magnesium (αvβ6) or DPBS 1% BSA (antibody, peptide loading) for 20 min at 4 °C or room temperature, respectively. For cells labeled with nanocages, cells were stained with SpyTag-Rhodamine diluted in DPBS for 1 h at room temperature. After secondary staining, cells were washed twice as before and resuspended in 200 μL DPBS 0.1% PFA before running on an Attune N×T Flow Cytometer (Life Technologies). Data was analyzed and plotted in FlowJo v10 software and MFI of singlet live cell events were used as measurements of binding or loading. The background of a secondary only control was subtracted from MFI values before they were normalized to either a positive control (A20FMDV2) or the highest observed value to account for machine-variability across experiments. GraphPad Prism 6 software was used to generate binding and loading curves and their associated apparent K_{D} and Kₘ values.

### Lentivirus Titering

H9 cells (10⁵) were transduced with 0, 0.05, 0.1, 0.25, 0.5, 1, 3, and 6 μL lentivirus in 0.5 mL complete RPMI with 5 μg/mL polybrene in a 24-well plate. After 24 h, 1 mL complete RPMI was added to cells to dilute the polybrene. The cells were stained with biotinylated Erbitux and SpyTag003(D2) 96 h post-transduction to measure the percentage of EGFRt and CAR positive cells, respectively. Titers were calculated from virus dilutions that gave percent positive cells in the linear titering range (10-45%) using the following equation:

$$\frac{TU}{mL} = \frac{( \% {EGFRt}^{+} or {CAR}^{+} ) ( {10}^{5} cells )}{mL of lentivirus} \qquad ( 1 )$$

Leftover H9 cells with near-100% transduction were used to generate H9 SpyCatcher CAR and DB5 CAR cell lines used in early studies.

### MALDI-ToF MS Proteolytic Stability

Normal mouse serum was prepared in-house as previously described.⁵³ SpyTag003(D2) peptides were incubated and extracted from serum as previously described,⁵³ except peptides (10 mg/mL in H₂O) were also incubated 1:10 (v/V) in complete RPMI media from BxPC3 cultures spiked further with 10⁶ BxPC3 cells at 37 °C to evaluate any protease activity stemming from the cancer cells. For these peptide-cell mixtures, cells were pelleted prior to sampling peptide at specific timepoints for subsequent acetonitrile extraction and stability analysis. Peptide degradation was qualitatively assessed by MALDI-ToF MS, and mass spectrums at the different timepoints were plotted and aligned in FlexAnalysis software (Bruker). A Java program called stability.jar was used to predict sequences of degradation products based on their observed molecular weights.

### DB5 CAR T-Cell Manufacturing

CD4⁺ and CD8⁺ T-cells (5×10⁶ each) were thawed and separately stimulated 1:1 with Dynabeads Human T-Activator CD3/CD28 (Invitrogen) in 4 ml complete RPMI media with 50 U/mL rhIL-2 (Miltenyi, CD8⁺ T-cells), 5 ng/mL rhIL-7 (Miltenyi, CD4⁺ T-cells), and/or 0.5 ng/mL rhIL-15 (Miltenyi, CD4⁺ and CD8⁺ T-cells) in a 12-well plate. After 2 d (S₁D₂), the activated T-cells were individually split into four 10⁶ cell groups and each transduced with lentivirus encoding DB5 CARs of different spacer lengths at an MOI of 3 in 0.5 mL complete RPMI media with 5 μg/mL polybrene, with the exception of a mock group that did not receive lentivirus. After a 4-h incubation with the lentivirus at 37 °C, T-cells were diluted 1:4 in complete RPMI media containing the appropriate cytokines to dilute the polybrene. Thereafter, media exchanges were conducted every 2-3 d to replenish cytokines and cells were moved to larger culture vessels when they appeared visually dense with yellowing media. Lentivirus-transduced cells were selected with 50-100 nM methotrexate (Teva) starting 2 d after transduction (S₁D₄) for 10 d total. The activator beads were removed 9 d poststimulation (S₁D₉), and T-cells were stained for EGFRt and CAR expression 11 d poststimulation (S₁D₁₁) to assess transduction efficiency. T-cells were functionally evaluated in ICCS and cytotoxicity assays 18-21 d poststimulation (S₁D₁₈-S₁D₂₁) after sufficiently expanding, and remaining cells were banked for future in vivo studies.

### T-Cell ICCS Assay

K562, K562 αvβ6:mCherry, and BxPC3 cells were pre-labeled with 500 nM bifunctional peptide at 2×10⁶ cells/ml in DPBS with calcium and magnesium for 30 min at 4 °C. CD4⁺ T-cells were similarly pre-armed with the bifunctional peptide except the incubation was conducted at room temperature. After washing, T-cells (unarmed and pre-armed) and target cells (unlabeled and pre-labeled) were resuspended in complete RPMI and co-cultured at a 1:1 effector-to-target ratio with 5×10⁵ cells each in 100 μL in a 96-well U-bottom plate. A cell stimulation cocktail (Invitrogen) containing phorbol 12-myristate 13-acetate (PMA) and ionomycin was added to certain wells as a positive control according to the manufacturer's instructions. Cells were incubated for 5 h at 37 °C and a protease transport inhibitor cocktail (Invitrogen) was added to all wells 1 h into the incubation to prevent cytokine secretion. During the incubation, leftover pre-labeled target cells and pre-armed T-cells were stained with streptavidin Alexa Fluor 647 by flow cytometry as described earlier to confirm successful pre-labeling and pre-arming with the bifunctional peptide.

At the end of the 5-h incubation, cells were washed twice with 200 μl DPBS and then stained with 100 μL Zombie Violet (1:500) in DPBS for 15 min at room temperature. After live/dead staining, cells were washed twice with 200 μl DPBS and resuspended in 10 μL FcR Blocking Reagent (Miltenyi) for 10 min at room temperature. After blocking Fc receptors, cells were stained directly with FITC anti-human CD4 antibody (BioLegend, 1:50) in 50 μL DPBS for 20 min at room temperature. After extracellular staining, cells were washed twice with 200 μL DPBS and resuspended in 100 μL cold Cytofix/Cytoperm buffer (BD) for 20 min at 4 °C. After fixation and permeabilization, cells were washed twice with 200 μL cold 1× Perm/Wash buffer (BD) and stained with BV510 anti-human IFNγ antibody (BioLegend, 1:25), PE-Cyanine 7 anti-human TNFα antibody (Invitrogen, 1:100), and APC anti-human IL-2 antibody (Invitrogen, 1:100) in 50 μL 1× Perm/Wash buffer for 30 min at 4 °C. After intracellular staining, cells were washed twice with 200 μL cold 1× Perm/Wash buffer and resuspended in 200 μL DPBS for running on the cytometer. Single-stain controls were included for compensation and fluorescence minus one (FMO) controls were used for gating.

### Peptide Internalization Study

Trypsin-lifted BxPC3 cells were pre-stained with Zombie Violet as described above. Meanwhile, bifunctional peptide was diluted to 400 nM in DPBS with calcium and magnesium over ice. Cells were then washed at 4 °C with DPBS 1% BSA to neutralize the Zombie Violet, plated in a U-bottom 96-well plate (2×10⁵ per well) over ice, and stained with 100 μL 400 nM peptide solution for 20 min at 4 °C. Cells were then washed twice with 200 μL cold DPBS, resuspended in complete media on ice, and transferred to a 37 °C incubator at different times over a 60-min period to induce internalization. Afterwards, the cells were transferred back on ice to stop further internalization, washed twice with 200 μL cold DPBS to remove media, and incubated with 100 μL streptavidin Alexa Fluor 647 in DPBS (1:500) for 20 min at 4 °C. Cells were subsequently washed twice as before and resuspended in 200 μL DPBS 0.1% PFA for running on the cytometer. The MFI of remaining peptide bound to the cell surface was normalized to peptide binding MFI on cells without 37 °C incubation (no internalization control), and normalized data was fit to an exponential decay curve in GraphPad Prism 6 software.

### T-Cell Cytotoxicity Assay

CD8⁺ T-cells were pre-armed with bifunctional peptide and washed as was done before for the CD4⁺ T-cells in the ICCS assay. K562, K562 SpyTag003(L), K562 αvβ6:mCherry, and BxPC3 target cells were washed once with DPBS with calcium and magnesium and then pre-labeled with 0.5 μM CellTrace Far Red (Invitrogen) in the same buffer at 10⁶ cells/ml for 20 min at room temperature. After target labeling, excess dye was neutralized by adding equal volume of DPBS with calcium and magnesium supplemented with 1% BSA to the cells and incubating for another 5 min at room temperature. Dye-labeled target cells were then transferred into complete RPMI media at 10⁶ cells/mL for a 30-min incubation at 37 °C in a CO₂ incubator to limit leakage of the dye to T-cells during co-culture. For plating, T-cells and dye-labeled target cells were resuspended in fresh complete RPMI media before co-culturing at 9:1, 3:1, and 1:1 effector-to-target ratios with 5×10⁴ total target cells in 200 μL in a 96-well U-bottom plate. Co-cultures were then pelleted and allowed to incubate for 18 h at 37 °C. During the incubation, leftover pre-armed T-cells were stained with streptavidin Alexa Fluor 647 by flow cytometry as described earlier to confirm successful pre-arming with the bifunctional peptide.

At the end of the 18-h incubation, cells were washed once with 200 μL DPBS to remove excess serum and then stained with 100 μL Live-Dead Fixable Green (Invitrogen, 1:2000) in DPBS for 30 min at room temperature. After incubation, remaining dye was inactivated by adding 100 μL DPBS supplemented with 1% BSA directly to the live-dead stained cells and incubating them for another 5 min at room temperature. Wells with suspension target cells (K562 and derivative cell lines) were then washed with 200 μL DPBS, whereas wells with adherent target cells (BxPC3) were washed with 50 μL Accutase (Innovative Cell Tech) for 5 min at room temperature to detach live cells and then volumed up to 200 μL with DPBS to complete the wash. Cells were lastly resuspended in 200 μl DPBS 0.2% PFA and run on the cytometer to determine the percentage of Live-Dead Fixable Green⁺ dead cells within CellTrace Far Red⁺ target cells. Wells with only target cells were used to determine the amount of spontaneous cell death without T-cells. T-cell specific lysis was calculated using the following equation:

$$( \frac{\% Killing - \% Spontaneus Death}{100 \% - \% Spontaneous Death} ) \qquad ( 2 )$$

### Aptamer-Chimera Synthesis, Purification, and Characterization

DBCO-modified DNA aptamer was synthesized by Integrated DNA technologies. For preparation of the aptamer-peptide chimera, 20 μM DBCO-modified aptamer was reacted with 200 μM azide-SpyTag003(D2)-biotin in DPBS with calcium and magnesium for 24 h at 37 °C on a thermal shaker. After aptamer-peptide conjugation, the reaction mixture was lyophilized and resuspended in 0.3 M sodium acetate pH 7.0 for purification of the aptamer-peptide chimera by ethanol precipitation. As unconjugated peptide is soluble in ethanol, ethanol only precipitates unconjugated aptamer and the aptamer-peptide chimera, removing excess peptide. Precipitated aptamer-peptide chimera was resuspended in a wash buffer designed for aptamer folding, which is comprised of DPBS with calcium and magnesium further supplemented with 5 mM MgCl2 (Fisher) and 25 mM D-glucose (Sigma-Aldrich). The concentration of the resuspended aptamer-peptide chimera was determined by both a NanoDrop UV-Vis spectrophotometer (Thermo Fisher) and a QuantTag Biotin Quantification Kit.

To characterize the conjugation efficiency of the peptide to the aptamer, 500 ng 50 bp DNA ladder (Thermo Scientific), 100 nfig. g free aptamer, and 100 ng aptamer-peptide chimera were denatured in 1× loading dye (NEB) containing 4 M urea (Fisher) for 3 min at 70 °C and separated on a Novex 15% TBE-urea gel (Invitrogen) by urea-PAGE. The gel was stained with SYBR Gold (1:10000, Invitrogen) in TBE buffer (Thermo Scientifc) for 30 min at room temperature and imaged on a Xenogen IVIS Spectrum (PerkinElmer) with 500 nm excitation and 540 nm emission. Conjugation yield of the aptamer-peptide chimera was measured semi-quantitatively by measuring the reduction in free aptamer band intensity with Fiji/ImageJ (Schindelin, et al., Nature Methods 2012, 9 (7), 676-682).

Aptamer-peptide chimera folding and binding to Jurkat and K562 cells was performed as previously described (Cheng, et al., J. of the American Chemical Society 2022, 144 (30), 13851-13864). Loading of the aptamer-peptide chimera onto CD8⁺ DB5 CAR T-cells was carried out as mentioned earlier for the bifunctional peptide.

FIG. 18 illustrates peptide sequences used in the first Experimental Example.

### Second Experimental Example

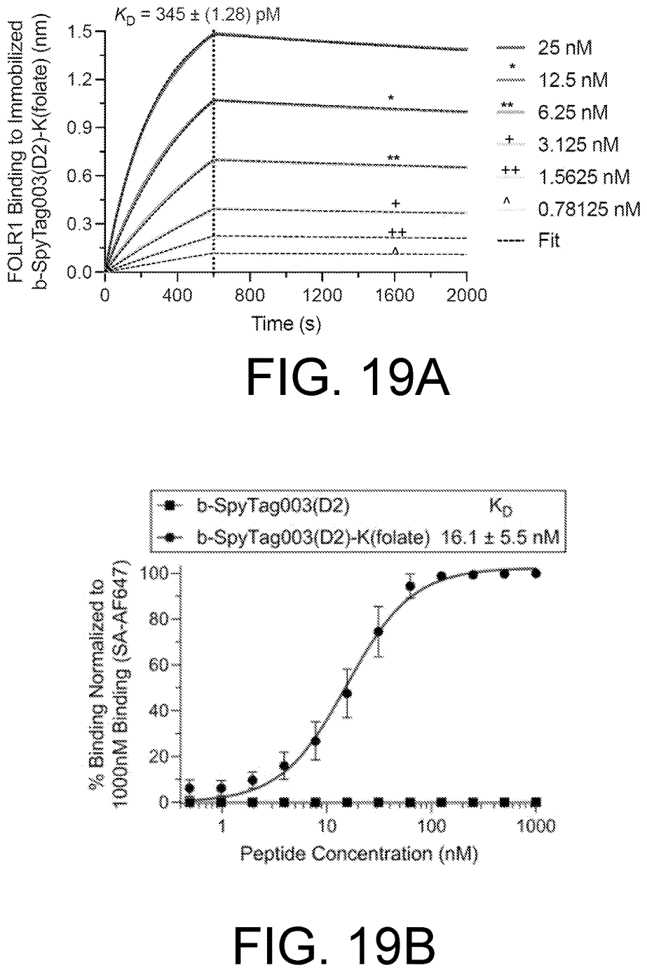

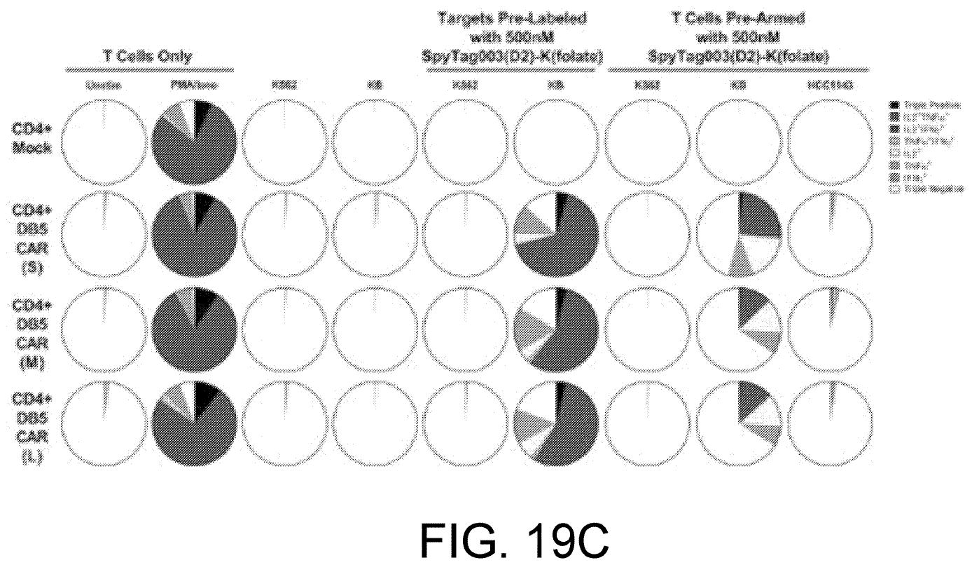

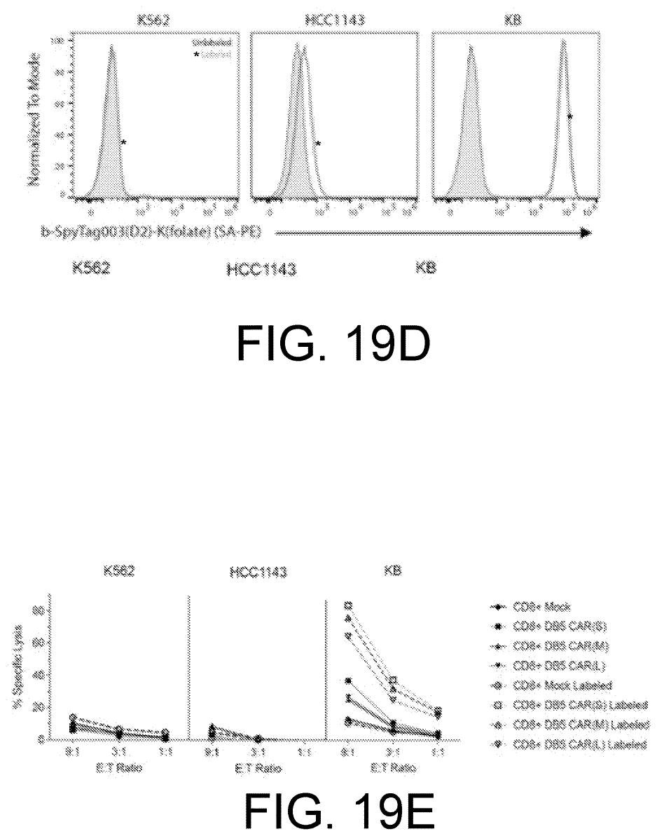

In this Experimental Example, biotinylated SpyTag003(D2)-K (folate) was loaded onto streptavidin glass sensors. The association and disassociation of FOLR1 protein onto the loaded sensors was measured over time at 25 °C with rapid mixing (≥2500 rpm). The binding kinetics for a dilution series of FOLR1 protein was used for a global fit analysis to determine the binding affinity of SpyTag003(D2)-K (folate). K_{D}=345±(1.28) pM, r²=0.9998. FIG. 19A illustrates results of the bifunctional folate peptide chimera bonding to FOLR1 protein, as measured by Biolayer interferometry.

FIG. 19B illustrates a fluorescence activated cell sorting analysis of b-SpyTag003(2D)-K (folate) binding to FOLR+KB cells in vitro. Data points and error bars represent mean binding percentage normalized to the highest concentration mean fluorescence intensity±SD; n=3. The curve represents nonlinear regression of specific binding with hill slope. Kd=16.1±5.5 nM.

FIGS. 19C-19E illustrate DB5 CAR T-cell responses with a bifunctional chimeric small molecule peptide-adapter. FIG. 19C shows results of flow cytometry histograms of target cell pre-labeling with 500 nM bifunctional folate-peptide (SpyTAg003(2D)-K (folate) on the day of the ICCS assay. ICCS pie charts of IL2, TNFα, and IFNγ cytokine production in CD4+DB5 CAR T cells after 4-h co-culture with target KB or HCC1143 cells. SpyTAg003(2D)-K (folate) was both pre-labeled on target cells and pre-armed on T cells for directing DB5 CAR T-cell responses against FOLR high- and low-expressing KB and HCC1143 cells, respectively. Pie charts are representative of 1 biological replicate.

FIG. 19D illustrates flow cytometry histograms of target cell pre-labeling with 500 nM bifunctional peptide-chimeral (bSpyTag003(2R)-K(folate)) on the day of the ICCS assay. Histograms are representative of 1 biological replicate. Peptide-chimera labeling is shown with lines labeled with stars.

FIG. 19E illustrates targeted killing of FOLR expressing cells after 18-h co-culture with CD8+DB5 CAR T target. The peptide-chimera was prelabeled on KB and HCC1143 cells incubated at 9:1, 3:1, and 1:1 effector-to-target cell ratios. Graphs are representative of target-cell lysis analyzed by flow cytometry of 1 biological replicate.

### Third Experimental Example

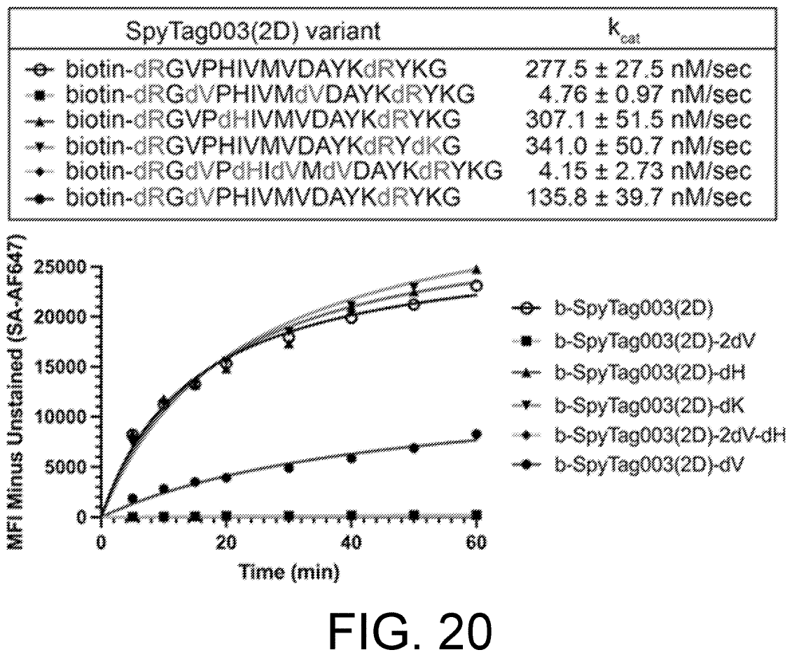

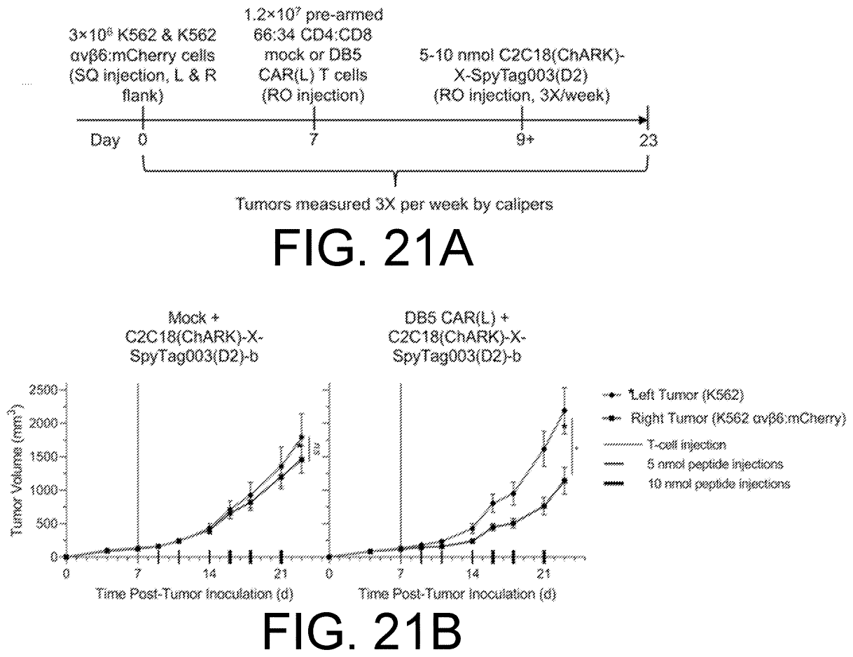

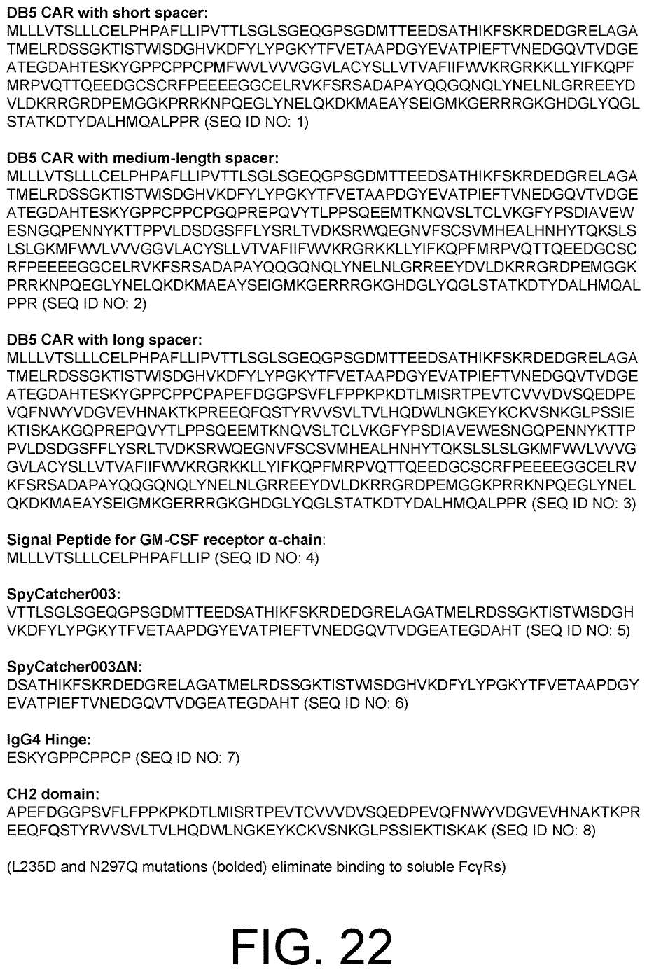

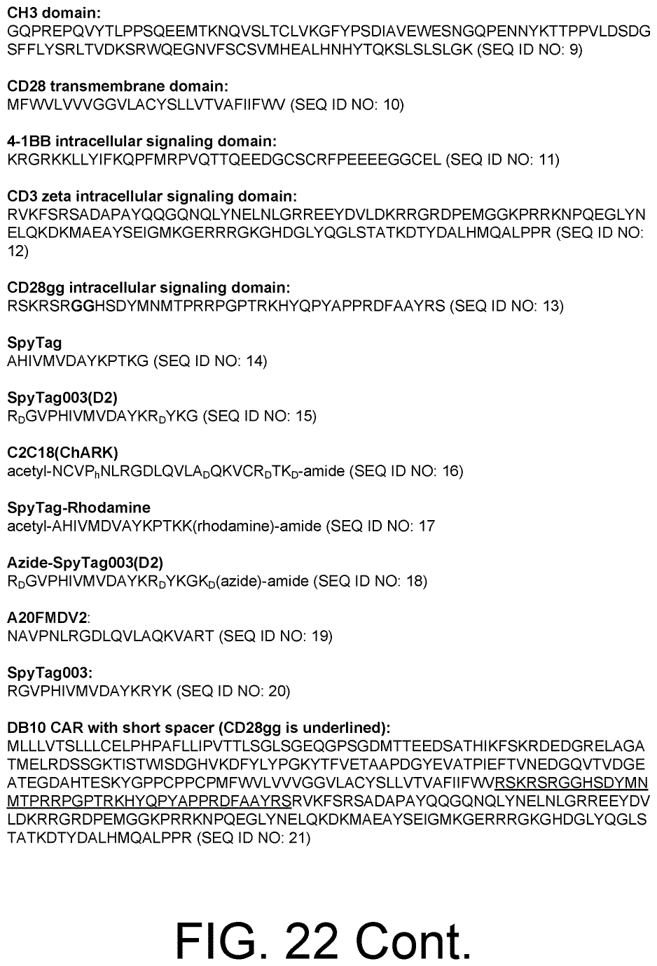

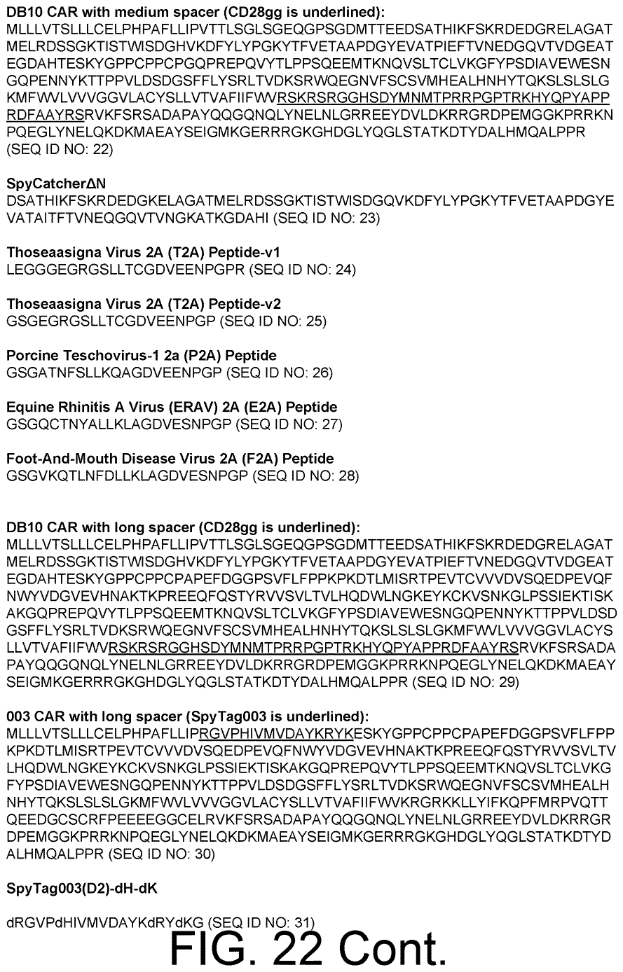

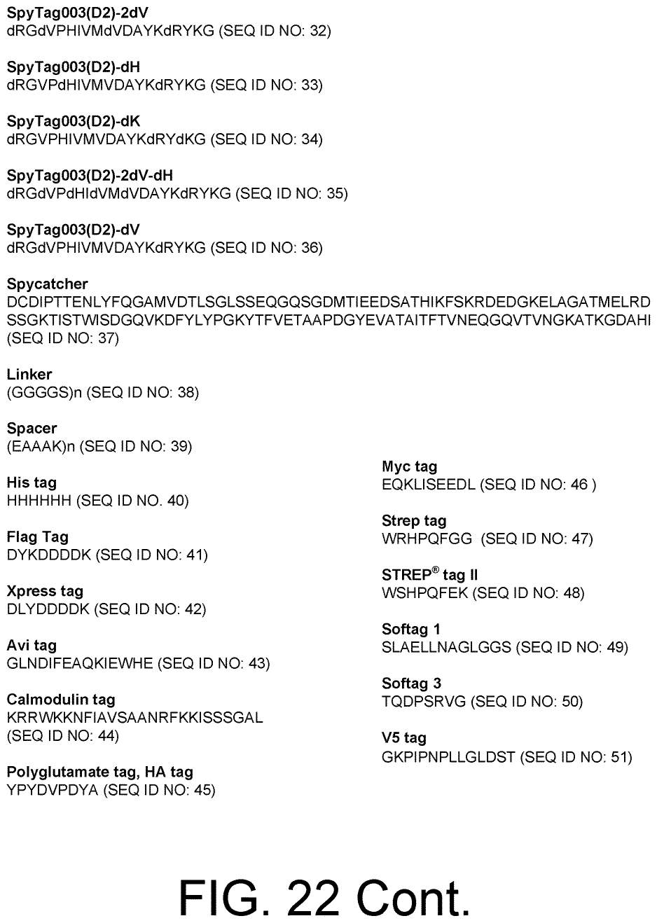

FIG. 20 illustrates loading kinetics for additional SpyTag003(2D) peptides (SEQ ID NO: 15, SEQ ID NO: 32, SEQ ID NO. 33, SEQ ID NO. 34, SEQ ID NO: 35, and SEQ ID NO: 36), which may have increased serum stability. A series of SpyTag003(3D) peptides with D-amino acid substitutions were synthesized. The loading kinetics of each peptide (100 nM) was examined with H9-DBS expressing cells over a 60 minute time course. FACS data was plotted and curve-fit with a nonlinear regression for Michaelis-Menten and kcat kinetics to quantify nM/sec loading.

### Fourth Experimental Example

In this Experimental Example, a bifunctional adaptor peptide and DB5 CAR T cells were shown to selectively reduce αvβ6+ tumor burden in a non-survival dual flank xenograft model. FIG. 20A illustrates the experimental protocol followed in the Fourth Experimental Example. NSG mice (8-12 weeks old) were subcutaneously (SQ) injected with 3×106 K562 and K562 αvβ6:mCherry cells in their left and right flank, respectively. After allowing the tumors to engraft for 7 days and reach a size of ~100 mm³, mice were retro-orbitally (RO) injected with either 1.2×107 pre-mixed CD4+/CD8+ mock or DB5 CAR(L) CAR T cells (66:34 CD4:CD8 ratio) that were pre-armed with 500 nM bifunctional peptide. Bifunctional peptide was continually re-dosed starting 2 days after T-cell injection at a frequency of 3 times per week via RO sinus with an initial dose at 5 nmol (3 total doses) before escalating to 10 nmol (additional 3 doses). Left and right flank tumor sizes were monitored by caliper measurements 3 times per week, and all mice were euthanized on day 23 post-tumor inoculation due to tumor burden. FIG. 20B illustrates corresponding tumor volume measurements over 23-day study comparing left (K562) and right (K562 αvβ6:mCherry) tumors in each group. Data points and error bars represent the mean±SEM; n=6-7 mice per group. ns>0.05, *P<0.05 (area under the curve analysis with two-sided unpaired t-test).

### Example Clauses

1. A method of administering a universal chimeric antigen receptor T (UCAR-T) cell therapy, the method including: administering, to a subject, engineered immune cells that express a chimeric antigen receptor (CAR) including an extracellular component and an intracellular component linked by a transmembrane domain, wherein the extracellular component includes an intermediate-binding domain; and administering, to the subject, a synthetic intermediate including a tag linked to an antigen binding domain, the tag specifically binding the intermediate-binding domain, the antigen binding domain specifically binding an antigen expressed on a surface of a target cell in the subject.
2. The method of clause 1, wherein the engineered immune cells include at least one of T cells, NK cells, macrophages, or hematopoietic stem cells.
3. The method of clause 1 or 2, wherein administering, to the subject, the engineered immune cells includes administering a formulation including the engineered immune cells intravenously.
4. The method of any of clauses 1 to 3, wherein the intermediate-binding domain has at least 90% sequence identity to a sequence as set forth in SEQ ID NO: 5, SEQ ID NO: 6, or SEQ ID NO: 20.
5. The method of any of clauses 1 to 4, wherein administering, to the subject, the synthetic intermediate includes administering a formulation including the synthetic intermediate intravenously.
6. The method of any of clauses 1 to 5, wherein administering, to the subject, the synthetic intermediate includes administering a formulation including the synthetic intermediate subcutaneously.
7. The method of any of clauses 1 to 6, wherein administering, to the subject, the synthetic intermediate includes administering an oral formulation including the synthetic intermediate.
8. The method of any of clauses 1 to 7, wherein the synthetic intermediate consists essentially of an unbranched peptide, a branched peptide, a DNA aptamer-peptide chimera, or a small molecule-peptide chimera.
9. The method of any of clauses 1 to 8, wherein the synthetic intermediate includes an unbranched peptide, a branched peptide, a DNA aptamer, a DNA aptamer-peptide chimera, a small molecule, a small molecule-peptide chimera, or a co-polymer formulation.
10. The method of any of clauses 1 to 9, wherein the synthetic intermediate includes a co-polymer formulation having a mass in a range of about 0.5 kDa to about 200 kDa.
11. The method of any of clauses 1 to 10, wherein the synthetic intermediate has a mass in a range of about 0.5 kDa to about 30 kDa.
12. The method of any of clauses 1 to 11, wherein the tag has at least 90% sequence identity to a sequence as set forth in SEQ ID NO: 5, SEQ ID NO: 6, SEQ ID NO: 15, SEQ ID NO: 20, SEQ ID NO: 31, SEQ ID NO: 32, SEQ ID NO: 33, SEQ ID NO: 34, SEQ ID NO: 35, or SEQ ID NO: 36.
13. The method of any of clauses 1 to 12, wherein the tag has at least 90% sequence identity to a sequence as set forth in SEQ ID NO: 15.
14. The method of any of clauses 1 to 13, wherein the synthetic intermediate includes multiple copies of the tag and/or multiple copies of the antigen binding domain.
15. The method of any of clauses 1 to 14, wherein the antigen includes DLL3, CLL1, GPRC5D, EpCAM, CD3, CD5, CD7, fibroblast activation protein, GAD65, citrullinated vimentin, myelin oligodendrocyte glycoprotein, carcinoembryonic antigen, prostate specific antigen, Prostate Stem Cell antigen, PSMA, Her2/neu, estrogen receptor, progesterone receptor, ephrinB2, CD19, CD20, CD22, CD23, CD123, CS-1, CE7, ROR1, mesothelin, c-Met, GD-2, EGFR, EGFRvIII, EphA2, IL 13Ra2, L1CAM, oaGD2, GD2, B7H3, CD33, VAR2CSA, MUC16, PD-L1, ERBB2, folate receptor, biotin receptor, CD56; glypican-2, disialoganglioside, EpCam, L1-CAM, Lewis Y, WT-1, Tyrosinase related protein 1, GD2, B-cell maturation antigen, CD24, SV40 T, carboxy-anhydrase-IX, or CD133.
16. The method of clause 15, wherein the target cell includes at least one of a cancer cell or an infected cell.
17. The method of any of clauses 1 to 16, wherein the target cell is disposed in a solid tumor of the subject.
18. The method of any of clauses 1 to 17, wherein administering, to the subject, the synthetic intermediate includes administering, do the subject, a first dosage of the synthetic intermediate, and wherein the method further includes administering, to the subject, a second dosage of the synthetic intermediate.
19. The method of clause 18, further including: identifying a response of the subject to the first dosage of the synthetic intermediate; and determining the second dosage of the synthetic intermediate based on the response.
20. The method of clause 19, wherein the response includes cytokine release syndrome (CRS), and wherein the second dosage is lower than the first dosage.
21. The method of any of clauses 1 to 20, further including: identifying a response of the subject to the synthetic intermediate; and in response to identifying the response of the subject to the synthetic intermediate, administering, to the subject, a inhibitory construct including the tag.
22. The method of any of clauses 1 to 21, the synthetic intermediate being a first synthetic intermediate, the tag being a first instance of the tag, the antigen binding domain being a first antigen binding domain, the antigen being a first antigen, the target cell being a first target cell, the method further including: administering, to the subject, a second synthetic intermediate including a second instance of the tag linked to a second antigen binding domain, the second antigen binding domain specifically binding a second antigen expressed by a second target cell in the subject.
23. The method of any of clauses 1 to 22, further including: determining that the target cell expressing the antigen is in the subject.
24. The method of clause 23, wherein determining that the target cell expressing the antigen is in the subject includes performing a genetic test or histological assay on a sample obtained from the subject.
25. The method of any of clauses 1 to 24, wherein the engineered immune cells are derived from immune cells obtained from the subject or a donor who is not the subject.
26. The method of clause 1, wherein the engineered immune cells are derived from iPSCs or CD34 hematopoietic stem cells.
27. A synthetic intermediate including a tag linked to an antigen binding domain.
28. The synthetic intermediate of clause 27, wherein the tag has a sequence as set forth in SEQ ID NO: 20 or a sequence having at least 90% sequence identity to the sequence as set forth in SEQ ID NO: 20.
29. The synthetic intermediate of clause 27 or 28, wherein the tag has a sequence as set forth in SEQ ID NO: 15 or a sequence having at least 90% sequence identity to the sequence as set forth in SEQ ID NO: 15.
30. The synthetic intermediate of any of clauses 27 to 29, wherein the tag has a sequence as set forth in SEQ ID NO: 31 or a sequence having at least 90% sequence identity to the sequence as set forth in SEQ ID NO: 31.
31. The synthetic intermediate of any of clauses 27 to 30, wherein the tag has a sequence as set forth in SEQ ID NO: 32 or a sequence having at least 90% sequence identity to the sequence as set forth in SEQ ID NO: 32.
32. The synthetic intermediate of any of clauses 27 to 31, wherein the tag has a sequence as set forth in SEQ ID NO: 33 or a sequence having at least 90% sequence identity to the sequence as set forth in SEQ ID NO: 33.
33. The synthetic intermediate of any of clauses 27 to 32, wherein the tag has a sequence as set forth in SEQ ID NO: 34 or a sequence having at least 90% sequence identity to the sequence as set forth in SEQ ID NO: 34.
34. The synthetic intermediate of any of clauses 27 to 33, wherein the tag has a sequence as set forth in SEQ ID NO: 35 or a sequence having at least 90% sequence identity to the sequence as set forth in SEQ ID NO: 35.
35. The synthetic intermediate of any of clauses 27 to 34, wherein the tag has a sequence as set forth in SEQ ID NO: 5 or a sequence having at least 90% sequence identity to the sequence as set forth in SEQ ID NO: 5.
36. The synthetic intermediate of any of clauses 27 to 35, wherein the tag has a sequence as set forth in SEQ ID NO: 6 or a sequence having at least 90% sequence identity to the sequence as set forth in SEQ ID NO: 6.
37. The synthetic intermediate of any of clauses 27 to 36, wherein the tag is truncated.
38. The synthetic intermediate of any of clauses 27 to 37, wherein the antigen binding domain includes at least one of a peptide, a DNA aptamer, or a small molecule.
39. The synthetic intermediate of any of clauses 27 to 38, wherein the antigen binding domain binds a cancer antigen or a viral antigen.
40. The synthetic intermediate of any of clauses 27 to 39, wherein the antigen binding domain binds an antigen associated with an autoimmune disorder or heart disease.
41. The synthetic intermediate of any of clauses 27 to 40, wherein the antigen binding domain includes an integrin αvβ6-specific peptide.
42. The synthetic intermediate of clause 41, wherein the integrin αvβ6-specific peptide includes C2C18(ChARK) or A20FMDV2.
43. The synthetic intermediate of clause 42, wherein the C2C18(ChARK) includes a sequence as set forth in SEQ ID NO: 16 or a sequence having at least 90% sequence identity to the sequence as set forth in SEQ ID NO: 16.
44. The synthetic intermediate of any of clauses 42 to 43, wherein the A20FMDV2 includes a sequence as set forth in SEQ ID NO: 19 or a sequence having at least 90% sequence identity to the sequence as set forth in SEQ ID NO: 19.
45. The synthetic intermediate of any of clauses 27 to 44, wherein the tag is linked to the antigen binding domain via branched peptide synthesis or conjugation chemistry.
46. The synthetic intermediate of clause 45, wherein the conjugation chemistry includes octyne-azide chemistry, alkyne-azide chemistry, BCN-azide chemistry, trans-cyclotene-tetrazine chemistry, copper-catalyzed azide-alkyne chemistry, carbodiimide crosslinker chemistry, or maleimide chemistry.
47. The synthetic intermediate of any of clauses 27 to 46, wherein the tag is linked to the antigen binding domain via a hexanoic acid-glycine linker.
48. A universal chimeric antigen receptor (CAR) that when expressed by a cell includes: an extracellular component and an intracellular component linked by a transmembrane domain, wherein the extracellular component includes an intermediate binding domain that specifically binds a tag in a synthetic intermediate, the intermediate binding domain having a sequence that has at least 90% sequence identity to a sequence set forth in SEQ ID NO: 5, SEQ ID NO: 6, or SEQ ID NO: 20.
49. The universal CAR of clause 48, wherein the extracellular component has the sequence as set forth in SEQ ID NO: 20.
50. The universal CAR of clause 48 or 49, wherein the extracellular component has the sequence as set forth in SEQ ID NO: 5.
51. The universal CAR of any of clauses 48 to 50, wherein the extracellular component has the sequence as set forth in SEQ ID NO: 6.
52. The universal CAR of any of clauses 48 to 51, wherein the extracellular component further includes a spacer region.
53. The universal CAR of clause 52, wherein the spacer region includes a short spacer, a medium-length spacer, or long spacer.
54. The universal CAR of clause 53, wherein the short spacer includes an IgG4 hinge.
55. The universal CAR of clause 53 or clause 54, wherein the medium-length spacer includes an IgG4 hinge and a CH3 domain.
56. The universal CAR of any of clauses 53 to 55, wherein the long spacer includes an IgG4 hinge, a CH2 domain, and a CH3 domain.
57. The universal CAR of any of clauses 52 to 56, wherein the spacer region includes an IgG4 hinge that includes the sequence as set forth in SEQ ID NO: 7 or a sequence having at least 90% sequence identity to the sequence as set forth in SEQ ID NO: 7.
58. The universal CAR of any of clauses 52 to 57, wherein the spacer region includes a CH2 domain that includes the sequence as set forth in SEQ ID NO: 8 or a sequence having at least 90% sequence identity to the sequence as set forth in SEQ ID NO: 8.
59. The universal CAR of any of clauses 52 to 58, wherein the spacer region includes a CH3 domain that includes the sequence as set forth in SEQ ID NO: 9 or a sequence having at least 90% sequence identity to the sequence as set forth in SEQ ID NO: 9.
60. The universal CAR of any of clauses 48 to 59, wherein the transmembrane domain includes a CD28 transmembrane domain.
61. The universal CAR of clause 60, wherein the CD28 transmembrane domain includes the sequence as set forth in SEQ ID NO: 10 or a sequence having at least 90% sequence identity to the sequence as set forth in SEQ ID NO: 10.
62. The universal CAR of any of clauses 48 to claim 61, wherein the intracellular component includes a CD3ζ intracellular signaling domain.
63. The universal CAR of clause 62, wherein the CD3ζ intracellular signaling domain includes the sequence as set forth in SEQ ID NO: 12 or a sequence having at least 90% sequence identity to the sequence as set forth in SEQ ID NO: 12.
64. The universal CAR of any of clauses 48 to 63, wherein the intracellular component includes a 4-1BB intracellular signaling domain.
65. The universal CAR of clause 64, wherein the 4-1BB intracellular signaling domain includes the sequence as set forth in SEQ ID NO: 11 or a sequence having at least 90% sequence identity to the sequence as set forth in SEQ ID NO: 11.
66. The universal CAR of any of clauses 48 to 65, wherein the intracellular component includes a CD28gg intracellular signaling domain.
67. The universal CAR of clause 66, wherein the CD28gg intracellular signaling domain includes the sequence as set forth in SEQ ID NO: 13 or a sequence having at least 90% sequence identity to the sequence as set forth in SEQ ID NO: 13.
68. The universal CAR of any of clauses 48 to 67, wherein the intracellular component includes a 4-1BB intracellular signaling domain and a CD34 intracellular signaling domain.
69. The universal CAR of any of clauses 48 to 68, wherein the intracellular component includes a CD28gg intracellular signaling domain and a CD3ζ intracellular signaling domain.
70. The universal CAR of any of clauses 48 to 69, wherein the extracellular component has a sequence that has at least 90% sequence identity to SEQ ID NO: 6, the intracellular component includes a 4-1BB intracellular signaling domain and a CD3ζ intracellular signaling domain, and the transmembrane domain includes a CD28 transmembrane domain.
71. The universal CAR of any of clauses 48 to 70, wherein the extracellular component has a sequence that has at least 90% sequence identity to SEQ ID NO: 6, the intracellular component includes a CD28gg intracellular signaling domain and a CD3ζ intracellular signaling domain, and the transmembrane domain includes a CD28 transmembrane domain.
72. The universal CAR of any of clauses 48 to 71, having the sequence as set forth in SEQ ID NO: 1 or a sequence having at least 90% sequence identity to the sequence as set forth in SEQ ID NO: 1.
73. The universal CAR of any of clauses 48 to 72, having the sequence as set forth in SEQ ID NO: 2 or a sequence having at least 90% sequence identity to the sequence as set forth in SEQ ID NO: 2.
74. The universal CAR of any of clauses 48 to 73, having the sequence as set forth in SEQ ID NO: 3 or a sequence having at least 90% sequence identity to the sequence as set forth in SEQ ID NO: 3.
75. The universal CAR of any of clauses 48 to 74, having the sequence as set forth in SEQ ID NO: 21 or a sequence having at least 90% sequence identity to the sequence as set forth in SEQ ID NO: 21.
76. The universal CAR of any of clauses 48 to 75, having the sequence as set forth in SEQ ID NO: 22 or a sequence having at least 90% sequence identity to the sequence as set forth in SEQ ID NO: 22.
77. The universal CAR of any of clauses 48 to 76, having the sequence as set forth in SEQ ID NO: 29 or a sequence having at least 90% sequence identity to the sequence as set forth in SEQ ID NO: 29.
78. The universal CAR of any of clauses 48 to 77, having the sequence as set forth in SEQ ID NO: 30 or a sequence having at least 90% sequence identity to the sequence as set forth in SEQ ID NO: 30.
79. The universal CAR of any of clauses 48 to 78, further encoding a selection marker.
80. The universal CAR of any of clauses 48 to 79, further encoding a transduction marker.
81. The universal CAR of any of clauses 48 to 80, further encoding a cleavable linker.
82. The universal CAR of clause 81, wherein the cleavable linker includes P2A, T2A, E2A, or F2A.
83. A nucleic acid molecule encoding the universal CAR of any of clauses 48 to 82.
84. A vector including the nucleic acid molecule of clause 83.
85. A formulation including a therapeutically effective amount of the vector of clause 84.
86. A cell genetically modified to express the universal CAR of any of clauses 48 to 82.
87. A formulation including a therapeutically effective amount of the cell of clause 86.
88. The cell of clause 86 or 87, wherein the cell is an immune cell.
89. The cell of clause 88, wherein the immune cell is a T-cell, B cell, natural killer cell, or macrophage.
90. The cell of clause 89, wherein the T-cell is a CD4+ or a CD8+ T-cell.
91. A system for directing a universal CAR-expressing T-cell response to a select target including: a cell genetically modified to express a universal CAR; and a synthetic intermediate including a tag linked to an antigen binding domain that binds the select target.
92. A kit, including: a nucleic acid molecule encoding a universal CAR; and a synthetic intermediate including a tag linked to an antigen binding domain.
93. A kit, including: a cell genetically modified to express a universal CAR; and a synthetic intermediate including a tag linked to an antigen binding domain.
94. A kit, including: a lentiviral vector encoding a universal CAR; and a synthetic intermediate including a tag linked to an antigen binding domain.
95. A method of treating a subject in need thereof, the method including: administering a formulation that includes or results in a genetically modified cell including a CAR including a binding domain; and administering a therapeutically effective amount of a synthetic intermediate, wherein the synthetic intermediate includes an antigen binding domain that binds a select target and a tag that covalently binds the binding domain of the CAR, thereby treating the subject in need thereof.
96. The method of clause 95, wherein the method further includes stopping administration of the synthetic intermediate to reduce a side effect of formulation administration.
97. The method of clause 95 or 96, wherein the method further includes administering a tag that covalently binds the binding domain of the CAR, thereby reducing a side effect of formulation administration.
98. The method of any of clauses 95 to 97, wherein the method further includes stopping administration of the synthetic intermediate when the subject is no longer in need thereof.
99. The method of any of clauses 95 to 98, wherein the method further includes readministering the synthetic intermediate when the subject is in need thereof.
100. The method of any of clauses 95 to 99, wherein the subject is in need thereof due to cancer or a viral infection.
101. The method of clause 100, wherein the cancer includes a solid tumor.
102. The method of any of clauses 95 to 101, wherein the select target is a cancer antigen or viral antigen.
103. The method of any of clauses 95 to 102, wherein the select target is associated with an autoimmune disorder or heart disease.
104. The method of any of clauses 95 to 103, wherein the antigen includes DLL3, CLL1, GPRC5D, EpCAM, CD3, CD5, CD7, fibroblast activation protein, GAD65, citrullinated vimentin, myelin oligodendrocyte glycoprotein, carcinoembryonic antigen, prostate specific antigen, Prostate Stem Cell antigen, PSMA, Her2/neu, estrogen receptor, progesterone receptor, ephrinB2, CD19, CD20, CD22, CD23, CD123, CS-1, CE7, ROR1, mesothelin, c-Met, GD-2, EGFR, EGFRvIII, EphA2, IL 13Ra2, L1CAM, oaGD2, GD2, B7H3, CD33, VAR2CSA, MUC16, PD-L1, ERBB2, folate receptor, biotin receptor, CD56; glypican-2, disialoganglioside, EpCam, L1-CAM, Lewis Y, WT-1, Tyrosinase related protein 1, GD2, B-cell maturation antigen, CD24, SV40 T, carboxy-anhydrase-IX, or CD133.
105. The method of any of clauses 95 to 104, wherein the method further includes administering a second type of synthetic intermediate wherein the second type of synthetic intermediate includes a second type of antigen binding domain that binds a second target.
106. The method of any of clauses 95 to 105, wherein the method further includes administering a plurality of different types of synthetic intermediates wherein each different type of synthetic intermediate includes a different type of antigen binding domain that binds a different target.
107. The method of clause 106, wherein the plurality of different types of synthetic intermediates can be administered simultaneously or at different times.
108. A device configured to administer, to a subject, a therapeutically effective dosage of a synthetic intermediate including a tag linked to an antigen binding domain.
109. The device of clause 108, wherein the device is an implantable device.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing implementations of the disclosure in diverse forms thereof.

As will be understood by one of ordinary skill in the art, each implementation disclosed herein can comprise, consist essentially of or consist of its particular stated element, step, or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the implementation to the specified elements, steps, ingredients or components and to those that do not materially affect the implementation. As used herein, the term “based on” is equivalent to “based at least partly on,” unless otherwise specified.

Unless otherwise indicated, all numbers expressing quantities, properties, conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The terms “a,” “an,” “the,” and similar referents used in the context of describing implementations (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate implementations of the disclosure and does not pose a limitation on the scope of the disclosure. No language in the specification should be construed as indicating any non-claimed element essential to the practice of implementations of the disclosure.

Groupings of alternative elements or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ile, Phe, Met, and Trp; Group 11 (aliphatic): Gly, Ala, Val, Leu, and Ile; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like. As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, 80% sequence identity, 85% sequence, 90% sequence identity, 95% sequence identity, 96% sequence identity, 97% sequence identity, 98% sequence identity, or 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y., Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42 °C in a solution including 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at 50 °C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 °C in a solution including 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 °C with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular implementations, “high affinity” binding domains refer to those binding domains with a Ka of at least 107 M-1, at least 108 M-1, at least 109 M-1, at least 1010 M-1, at least 1011 M-1, at least 1012 M-1, or at least 1013 M-1. In particular implementations, “low affinity” binding domains refer to those binding domains with a Ka of up to 107 M-1, up to 106 M-1, up to 105 M-1. Alternatively, affinity may be defined as an equilibrium dissociation constant (Kd) of a particular binding interaction with units of M (e.g., 10-5 M to 10-13 M). In certain implementations, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

Certain implementations are described herein, including the best mode known to the inventors for carrying out implementations of the disclosure. Of course, variations on these described implementations will become apparent to those of ordinary skill in the art upon reading the foregoing description. The inventor expects skilled artisans to employ such variations as appropriate, and the inventors intend for implementations to be practiced otherwise than specifically described herein. Accordingly, the scope of this disclosure includes all modifications and equivalents of the subject matter recited in the claims appended hereto as permitted by applicable law. Moreover, any combination of the above-described elements in all possible variations thereof is encompassed by implementations of the disclosure unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the implementations of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred implementations of the present invention only and are presented in the cause of providing what is believed to be the most useful and readily understood description of the principles and conceptual aspects of various implementations of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

1. A method of administering a universal chimeric antigen receptor T (UCAR-T) cell therapy, the method comprising:
administering, to a subject, engineered immune cells that express a chimeric antigen receptor (CAR) comprising an extracellular component and an intracellular component linked by a transmembrane domain, wherein the extracellular component comprises an intermediate-binding domain; and
administering, to the subject, a synthetic intermediate comprising a tag linked to an antigen binding domain, the tag covalently and specifically binding the intermediate-binding domain, the antigen binding domain specifically binding an antigen expressed on a surface of a target cell in the subject.

2-3. (canceled)

4. The method of claim 1, wherein the intermediate-binding domain has at least 90% sequence identity to the sequence as set forth in SEQ ID NO: 5 or SEQ ID NO: 6.

5. (canceled)

6. The method of claim 1, wherein administering, to the subject, the synthetic intermediate comprises:
administering a subcutaneous formulation comprising the synthetic intermediate; and/or
administering an oral formulation comprising the synthetic intermediate.

7-8. (canceled)

9. The method of claim 1, wherein the synthetic intermediate comprises an unbranched peptide, a branched peptide, a DNA aptamer-peptide chimera, a small molecule-peptide chimera, or a co-polymer formulation.

10. The method of claim 1, wherein the synthetic intermediate comprises a co-polymer formulation having a mass in a range of about 0.5 kDa to about 200 kDa.

11. The method of claim 1, wherein the synthetic intermediate has a mass in a range of about 0.5 kDa to about 30 kDa.

12. The method of claim 1, wherein the tag has at least 90% sequence identity to the sequence as set forth in SEQ ID NO: 15, SEQ ID NO: 20, SEQ ID NO: 31, SEQ ID NO: 32, SEQ ID NO: 33, SEQ ID NO: 34, SEQ ID NO: 35, or SEQ ID NO: 36.

13. The method of claim 1, wherein the tag has at least 90% sequence identity to the sequence as set forth in SEQ ID NO: 15 or SEQ ID NO: 34.

14-17. (canceled)

18. The method of claim 1, wherein administering, to the subject, the synthetic intermediate comprises administering, do the subject, a first dosage of the synthetic intermediate,
wherein the method further comprises administering, to the subject, a second dosage of the synthetic intermediate, and
wherein the method further comprises:
identifying that the subject has cytokine release syndrome (CRS) as a response to the first dosage of the synthetic intermediate; and
based on identifying that the subject has CRS as the response to the first dosage of the synthetic intermediate, determining the second dosage of the synthetic intermediate to be lower than the first dosage.

19-20. (canceled)

21. The method of claim 1, further comprising:
identifying a response of the subject to the synthetic intermediate; and
in response to identifying the response of the subject to the synthetic intermediate, administering, to the subject, a inhibitory construct comprising the tag.

22. The method of claim 1, the synthetic intermediate being a first synthetic intermediate, the tag being a first instance of the tag, the antigen binding domain being a first antigen binding domain, the antigen being a first antigen, the target cell being a first target cell, the method further comprising:
administering, to the subject, a second synthetic intermediate comprising a second instance of the tag linked to a second antigen binding domain, the second antigen binding domain specifically binding a second antigen expressed by a second target cell in the subject.

23-25. (canceled)

26. The method of claim 1, wherein the engineered immune cells are derived from iPSCs or CD34 hematopoietic stem cells.

27-94. (canceled)

95. A method of treating a subject in need thereof, the method comprising:
administering a formulation that comprises or results in a genetically modified cell comprising a CAR comprising a binding domain; and
administering a therapeutically effective amount of a synthetic intermediate, wherein the synthetic intermediate comprises an antigen binding domain that binds a select target and a tag that covalently binds the binding domain of the CAR, thereby treating the subject in need thereof.

96. (canceled)

97. The method of claim 95, wherein the method further comprises administering a tag that covalently binds the binding domain of the CAR, thereby reducing a side effect of formulation administration.

98-99. (canceled)

100. The method of claim 95, wherein the subject is in need thereof due to cancer comprising a solid tumor.

101-102. (canceled)

103. The method of claim 95, wherein the select target is associated with an autoimmune disorder or heart disease.

104-105. (canceled)

106. The method of claim 95, wherein the method further comprises administering a plurality of different types of synthetic intermediates wherein each different type of synthetic intermediate includes a different type of antigen binding domain that binds a different target.

107-109. (canceled)

110. A method of administering a universal chimeric antigen receptor T (UCAR-T) cell therapy, the method comprising:
administering, to a subject, engineered immune cells that express a chimeric antigen receptor (CAR) comprising an extracellular component and an intracellular component linked by a transmembrane domain, wherein the extracellular component comprises an intermediate-binding domain; and
administering, to the subject, a synthetic intermediate consisting essentially of a peptide comprising:
a tag covalently and specifically binding the intermediate-binding domain of the extracellular component of the CAR; and
an antigen binding domain linked to the tag and specifically binding an antigen expressed on a surface of a target cell in the subject.

111. The method of claim 110, wherein the target cell comprises a cancer cell located in an interior cellular layer of a solid tumor of the subject.

112. The method of claim 110, wherein the tag has at least 90% sequence identity with the sequence as set forth in SEQ ID NO: 15, SEQ ID NO: 31, SEQ ID NO: 32, SEQ ID NO: 33, SEQ ID NO: 34, or SEQ ID NO: 35.
